(19) United States
(12) Patent Application Publication
(10) Pub. No.: US 2014/0303333 A1
(43) Pub. Date: Oct. 9, 2014

(54) IRON BISPHENOLATE COMPLEXES AND METHODS OF USE AND SYNTHESIS THEREOF

(71) Applicants: UNIVERSITY OF PRINCE EDWARD ISLAND, Charlottetown (CA); GENESIS GROUP INC., St. John's (NL)

(72) Inventors: Michael P. Shaver, Charlottetown (CA); Christopher M. Kozak, St. John's (CA)

(21) Appl. No.: 14/351,550

(22) PCT Filed: Oct. 15, 2012
(86) PCT No.: PCT/CA2012/000943

§ 371 Date: Apr. 11, 2014

Related U.S. Application Data
(60) Provisional application No. 61/547,426, filed on Oct. 14, 2011.

(30) Foreign Application Priority Data
Oct. 14, 2011 (CA) 2755147

(51) Int. Cl. C08F 4/80; C07D 213/38; C07C 213/02; C07C 217/08; C07F 15/02; C07C 215/50; C07D 307/14

(52) U.S. Cl. CPC C08F 4/80; C07C 215/50; C07D 213/38; C07D 307/14; C07C 217/08; C07F 15/025; C07C 213/02
USPC 526/147; 564/390; 546/334; 564/367; 549/492; 556/150; 526/172; 526/329.7; 526/346; 526/319; 502/164

(57) ABSTRACT

The present application, relates to iron bisphenolate complexes and methods of use and synthesis thereof. The iron complexes are prepared from tridentate or tetradentate ligands of Formula I: wherein R¹ and R² are as defined herein. Also provided are methods and processes of using the iron bisphenolate complexes as catalysts in cross-coupling reactions and in controlled radical polymerizations.

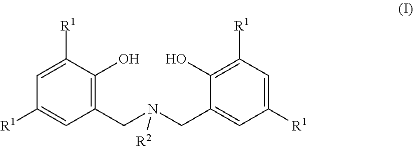

### FIELD OF THE INVENTION

The present invention pertains to bisphenolate complexes and their use as catalysts, for example, in carbon cross-coupling reactions and controlled radical polymerization reactions. More particularly, the present invention pertains to iron bisphenolate catalysts and catalyst systems useful for carbon cross coupling and controlled radical polymerization reactions, and to methods of synthesis thereof.

### BACKGROUND

Transition metal catalyzed Grignard cross-coupling is an important class of carbon-carbon bond forming reactions, including nickel- and palladium-catalyzed Kumada-Corriu couplings of Grignard reagents with organohalides. Cross-coupling methods are useful in modern organic synthesis and have found applications in industrial practice for the production of agrochemical, fine chemicals and pharmaceuticals.

Traditionally, cross-coupling reactions have been catalyzed by palladium, copper or nickel complexes, however, the use of these metal catalysts can have drawbacks, including, for example, high cost, potential toxicity from residual catalyst remaining in the products, and the requirement for specialized ligands to sufficiently activate the metal centre (United States Published Patent Application No. US 2009/0247764). Environmental concerns about the toxicity of heavy metal catalysts has also prompted the development of non-toxic, or less toxic, alternatives, and alternatives that can be used with non-toxic, or less toxic, solvents. Further, metal catalysts are often coloured, which can result in undesirable discolouration in products having residual catalyst.

In some instances, iron complexes have been identified as attractive alternatives to other metal based catalysts (e.g., transition metal complexes) used in cross-coupling reactions, at least in part due to the relative abundance and low cost of iron. Additionally, iron is stable, widely commercially available, and toxicologically benign compared to other metals used in catalysts for cross-coupling reactions, thereby reducing the need for recovery of the catalyst from reaction mixtures and chemical products. In the early 1970s, Kochi et al. demonstrated that iron salts could catalyze cross-coupling of vinyl halides with alkyl Grignard compounds (Kochi et al. J. Am Chem. Soc. Vol. 93, Iss. 6 pp. 1471, 1971). More recently, Nakamura et al. disclosed the use of iron halide catalysts in combination with a diamine compound (e.g., tetramethylethylenediamine (TMEDA)) for synthesizing alkyl-substituted aromatic compounds from an aromatic Grignard reagent and an alkyl halide (United States Patent Application Publication No. 2007/0123734).

Furstner et al. disclosed methods of cross-coupling various types of aromatic substrates with different iron complexes (U.S. Pat. No. 7,026,478). Sundermeier et al. also disclosed the cross-coupling of aryl Grignard reagents with vinyl halides using iron salts, especially iron halides (U.S. Patent Publication No. 2009/0247764). Reviews of carbon cross-coupling employing iron as a catalyst can be found by Jana et al. (Chem. Rev. 2011, Vol. 111, pp. 1417-1492) and Czaplik et al. (ChemSusChem 2009, Vol. 2, pp. 396-417).

Iron catalysts are complementary to Ni and Pd in that they can successfully couple alkyl halides with Grignard reagents, which is not easily achieved using Ni or Pd due to competing β-hydride elimination. However, unactivated alkyl halides, particularly alkyl chlorides, continue to pose a challenge and only a few examples of C—C bond formation using alkyl chlorides have been reported. Also, there have been few reports of the synthesis of diarylmethane compounds via iron-catalyzed coupling of aryl Grignards with benzyl halides, and others have found these products required the use of aryl zinc nucleophiles because aryl Grignard reagents proved unsatisfactory. There remains a need, therefore, for a catalyst system that can address these shortcomings.

The combination of iron with amine-bis(phenolate) ligands and their use as catalysts in carbon cross-coupling was investigated by Chowdhury et al. (Chem. Comm. 2008 pp. 96-98). Another example was disclosed by Groysman et al. (Organometallics, Vol. 23, No. 22, 2004). Groysman disclosed the use of amine-bis(phenolate) ligands in combination with zirconium, hafnium, and titanium to prepare catalysts for 1-hexene polymerization. Other titanium and zirconium catalysts have been prepared by the same group. (Gendler at al. Journal of Polymer Science: Part A: Polymer Chemistry, Vol. 44, pp. 1136-1146, 2006; Tshuva et al. Organometallics 2001, 20, 3017-3028; Groysman et al. Inorganica Chimica Acta Vol. 345, pp. 137-144, 2003). Other work by Yao et al. (Organometallics, 2005, 24 (16), pp 4014-4020) demonstrated that amine-bis(phenolate) lanthanide complexes could initiate polymerization of ε-caprolactone.

A series of catalysts having a tetradentate amine-bis(phenolate) ligand on an iron centre were prepared by Velusamy et al as structural and functional models for the intradiol cleaving catechol 1,2-dioxygenases (Velusamy et al. Inorg. Chem. 2003, 42, pp. 8283-8293). The iron complexes of Velusamy employ similar ligands to those disclosed by Groysman, however, Velusamy did not report any catalytic activity of these complexes.

Radical polymerization is a polymerization method by which a polymer is formed polymerization of free radical monomer units. In conventional radical polymerization, the propagation reaction is rapid and quickly produces high molecular weight polymers with broad molecular weight distributions. (Coessens V M C et al. 2010 Journal of Chemical Education, Vol. 8, No. 9, pp. 916-919). The properties and applications of a polymer depend on the molecular weight distribution and molecular structure of the polymer. Synthetic procedures that allow control over the composition, topology and functionality of the polymer are desirable for industrial use.

Controlled radical polymerization (CRP) is a radical polymerization method of manufacturing a polymer having a well-controlled molecular weight and structure with a narrow molecular weight distribution. The measurement of molecular weight distribution in a polymer is known as the polydispersity index (PDI). Atom transfer radical polymerization (ATRP) is one type of CRP. In ATRP, the polymerizing radical is intermittently inactivated, minimizing premature chain termination and allowing all polymer chains to propagate at approximately the same rate (id. p. 916). This method can provide a polymer having a predictable molecular weight as well as a narrow molecular weight distribution or low polydispersity index.

Traditional copper catalysts used in CRP often leave strong and persistent color in the polymer products. In addition, toxic copper residues are problematic in materials for human and animal use. Accordingly, there remains a need for catalysts for CRP and ATRP that provide polymer products in good yield with good polydispersity and minimal discolouration, wherein the catalysts and products are less toxic.

There have been a number of attempts to use iron catalysts in CRP, including, for example, Allan, L. E. N.; Shaver, M. P.; White, A. J. P.; Gibson, V. C. “Correlation of metal spin-state in α-diimine iron catalysts with polymerization mechanism.” Inorg. Chem. 2007, 46, 8963.; Shaver, M. P.; Allan, L. E. N.; Gibson, V. C. “Organometallic intermediates in the controlled radical polymerization of styrene by α-diimine iron catalysts.” Organometallics. 2007, 26, 4725; Shaver, M. P.; Allan, L. E. N.; Rzepa, H. S.; and Gibson, V. C. “Correlation of metal spin state with catalytic reactivity: Polymerizations mediated by α-diimine iron complexes.” Angew. Chem. Int. Ed. 2006, 45, 124. However, the polymerization reactions catalyzed by these complexes can be improved in terms of speed and/or control. Furthermore, the polymer products of CRP using these iron catalysts are pink rather than white (white is typically preferred).

Given the demand for simple and economical methods for carbon-carbon bond formation in polymer, pharmaceutical, agrochemical and fine chemical industries, there remains a need for effective, flexible, catalysts and catalyst systems for carbon coupling in both cross-coupling reactions and polymerization.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide iron bisphenolate complexes and methods of use and synthesis thereof.

In accordance with one aspect, there is provided a compound having the structure of Formula I:

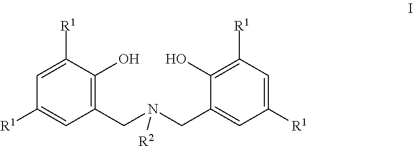

wherein

each R¹ is independently an electron withdrawing group, such as F, Cl, Br, I, CF₃, nitro, nitrile, carbonyl or a substituted carbonyl; and

R² is a substituted or unsubstituted C₁-C₂₅, or C₁ to C₁₀, linear, branched or cyclic alkyl, a substituted or unsubstituted non-aromatic heterocycle, a substituted or unsubstituted aryl, or a substituted or unsubstituted heteroaryl, and optionally comprises a coordinating atom, with the proviso that when the R¹ substituents are all Cl, R² cannot be CH₃, CH₂CH₂N(CH₃)₂, or CH₂CH₂OCH₃, and when the R¹ substituents are all Br, R² cannot be CH₃ or CH₂CH₂N(CH₃)₂.

The coordinating atom can be, for example, any Group 15 element (nitrogen, phosphorus, arsenic, antimony and bismuth) or Group 16 element (oxygen, sulfur, selenium, or tellurium) or a carbenic atom of a carbene-containing fragment (such as an N-heterocyclic carbene). In a specific embodiment, the coordinating atom is an aprotic N or O atom.

The compound of Formula I is useful as tridentate ligand or, when R² includes a coordinating atom, a tetradentate ligand suitable for iron complexation.

In accordance with one embodiment, each R¹ is independently halogen, such as F, Cl, Br or I, or CF₃, nitro, nitrile, carbonyl or a substituted carbonyl.

In accordance with another aspect, there is provided a method for synthesizing the compound of Formula I,

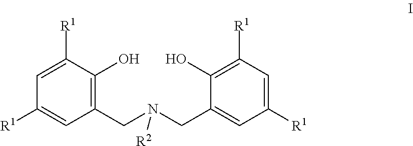

which comprises β-aminoalkylation of a phenol of Formula IV

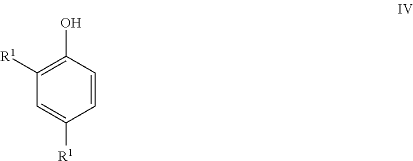

with formaldehyde and an amine of Formula V

H₂N—R² V

wherein

each R¹ is independently an electron withdrawing group, such as F, Cl, Br, I, CF₃, nitro, nitrile, carbonyl or a substituted carbonyl; and

R² is a substituted or unsubstituted C₁-C₂₅, or C₁ to C₁₀, linear, branched or cyclic alkyl, a substituted or unsubstituted non-aromatic heterocycle, a substituted or unsubstituted aryl, or a substituted or unsubstituted heteroaryl, and optionally comprises a coordinating atom, such as, an aprotic heteroatom in a heterocycle, heteroaryl or substituted alkyl group.

In accordance with one embodiment, each R¹ is independently halogen, such as F, Cl, Br or I, or CF₃, nitro, nitrile, carbonyl or a substituted carbonyl.

In one embodiment, the method for synthesizing the compound of Formula I comprises the reaction of Scheme 1:

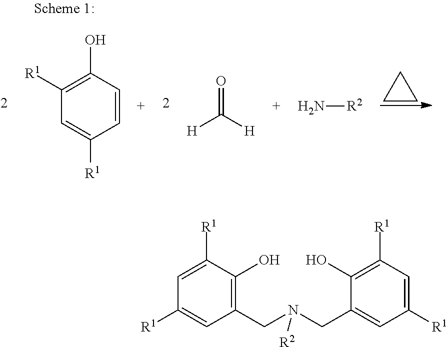

wherein

each R¹ is independently an electron withdrawing group; and

R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom. The coordinating atom can be, for example, any Group 15 element (nitrogen, phosphorus, arsenic, antimony and bismuth) or Group 16 element (oxygen, sulfur, selenium, or tellurium) or a carbenic atom of a carbene-containing fragment (such as an N-heterocyclic carbene). In a specific embodiment, the coordinating atom is an aprotic N or O atom.

In accordance with one embodiment, each R¹ is independently halogen, such as F, Cl, Br or I, or CF₃, nitro, nitrile, carbonyl or a substituted carbonyl.

In accordance with one embodiment, the reaction is carried out in water.

In accordance with one aspect, there is provided an iron complex having the structure of Formula II:

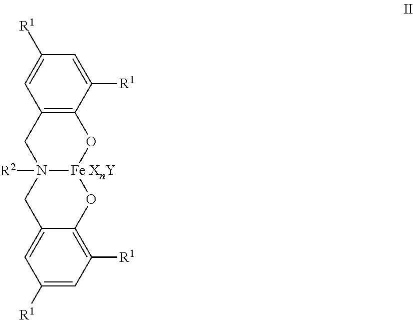

wherein:

each R¹ is independently an electron withdrawing group or a substituted or unsubstituted C₁-C₂₅, or C₁ to C₁₀, linear, branched or cyclic alkyl, or a substituted or unsubstituted aryl, where R¹ does not comprise a coordinating atom;

R² is a substituted or unsubstituted C₁-C₂₅, or C₁ to C₁₀, linear, branched or cyclic alkyl, a substituted or unsubstituted non-aromatic heterocycle, a substituted or unsubstituted aryl, or a substituted or unsubstituted heteroaryl, and optionally comprises a coordinating atom;

X is halogen, such as F, Cl, Br or I;

n is 1 or 2; and

Y is absent or a coordinating solvent molecule, such as water, methanol, ethanol, tetrahydrofuran (THF) or acetonitrile, or, when n is 2, Y is a negatively charged ionic species, such as an alkali metal or NR′″₄⁺, where R′″ is H, alkyl, aryl, heteroalkyl or heteroaryl; wherein when R² comprises a coordinating atom, the coordinating atom forms a dative bond to the Fe atom, and

with the proviso that when the R¹ substituents are all methyl, R² cannot be CH₂CH₂N(CH₃)₂, when the two R¹ substituents ortho to the oxygen are tert-butyl and the two R¹ substituents para to the oxygen are methyl, R² cannot be —CH₂pyridine, or CH₂CH₂N(CH₃)₂, and when R² is —CH₂(2-tetrahydrofuran) or —CH₂CH₂methoxy, R¹ is an electron withdrawing group.

In accordance with one embodiment of the compound of Formula II, the electron withdrawing group is F, Cl, Br, I, CF₃, nitro, nitrile, carbonyl or substituted carbonyl.

In accordance with one embodiment of the compound of Formula II, there is provided an iron complex having the structure of Formula IIa or IIa′:

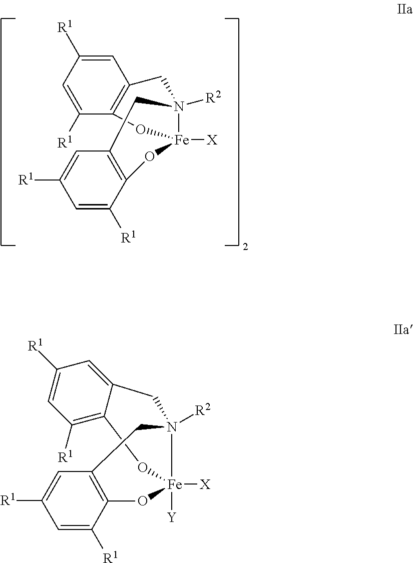

wherein:

R¹, R², X and Y are as defined above in relation to the compound of Formula II; and

wherein R² does not comprise a coordinating atom bound via a covalent dative bond to the Fe atom.

In accordance with another embodiment of the compound of Formula II, there is provided an iron complex having the structure of Formula IIb:

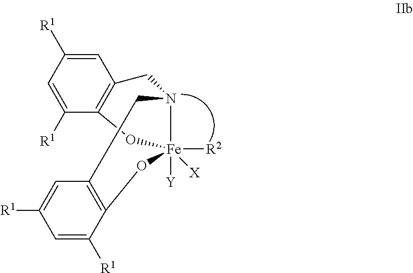

wherein:

R¹, R², X and Y are as defined above in relation to the compound of Formula II; and

wherein R² comprises a coordinating atom bound via a covalent dative bond to the Fe atom.

In one embodiment of the compound of Formula II, Y is absent. In an alternative embodiment Y is OH₂.

In accordance with another aspect, there is provided a catalyst system comprising the iron complex of Formula II, IIa. IIa′ or IIb. In one embodiment the catalyst system further comprising one or more solvents, reagents, initiators, stabilizers, or any combinations thereof.

In accordance with one aspect, there is provided a process for synthesizing an iron complex of Formula IIa or IIa′, which comprises reacting an amine-bis(phenolate) ligand of Formula I with an iron halide to give the catalyst of Formula II:

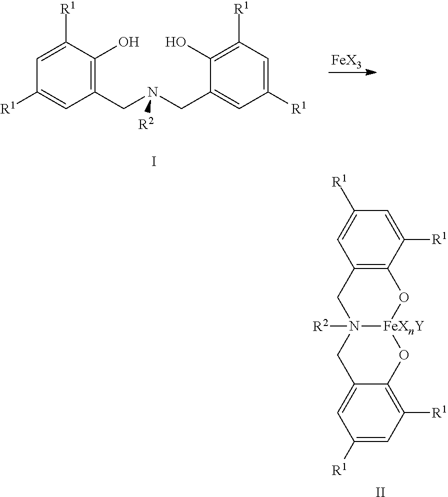

wherein
each R¹ is independently an electron withdrawing group or a substituted or unsubstituted C₁-C₂₅, or C₁ to C₁₀, linear, branched or cyclic alkyl, or a substituted or unsubstituted aryl, where R¹ does not comprise a coordinating atom;
R² is a substituted or unsubstituted C₁-C₂₅, or C₁ to C₁₀, linear, branched or cyclic alkyl, a substituted or unsubstituted non-aromatic heterocycle, a substituted or unsubstituted aryl, or a substituted or unsubstituted heteroaryl, and optionally comprises a coordinating atom;
X is halogen, such as F, Cl, Br or I;
n is 1 or 2; and
Y is absent or a coordinating solvent molecule, such as water, methanol, ethanol, tetrahydrofuran (THF) or acetonitrile, or, when n is 2, Y is a negatively charged ionic species, such as an alkali metal or NR′″₄⁺, where R′″ is H, alkyl, aryl, heteroalkyl or heteroaryl;
   wherein when R² comprises a coordinating atom, the coordinating atom forms a dative bond to the Fe atom, and
   with the proviso that when R¹ is methyl, R² cannot be C₂H₄N(CH₃)₂, and that when R² is CH₂(2-tetrahydrofuran) or CH₂CH₂methoxy, R¹ is an electron withdrawing group.

In accordance with an embodiment, there is provided a process for synthesizing an iron complex of Formula IIa or IIa′, which comprises reacting a tridentate amine-bis(phenolate) ligand of Formula I with an iron halide to give the catalyst of Formula IIa or IIa′:

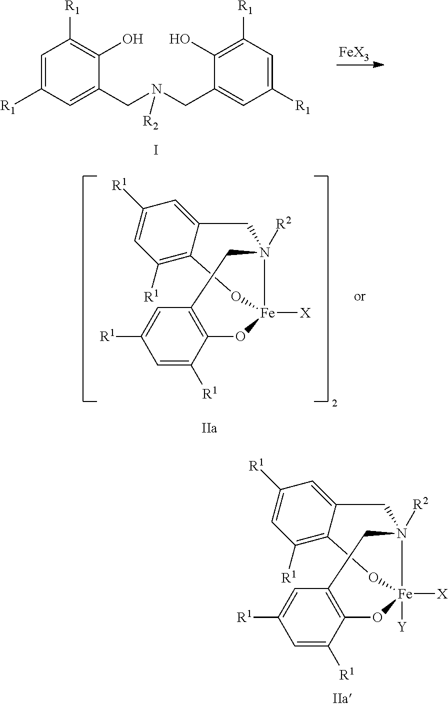

wherein:

R¹, R², X and Y are as defined above in relation to the compound of Formula II; and

wherein R² does not comprise a coordinating atom coordinated to the Fe atom.

In accordance with another embodiment, there is provided a process for synthesizing a metal complex of Formula IIb, which comprises reacting a tetradentate amine-bis(phenolate) ligand of Formula I with an iron halide to give the catalyst of Formula IIb:

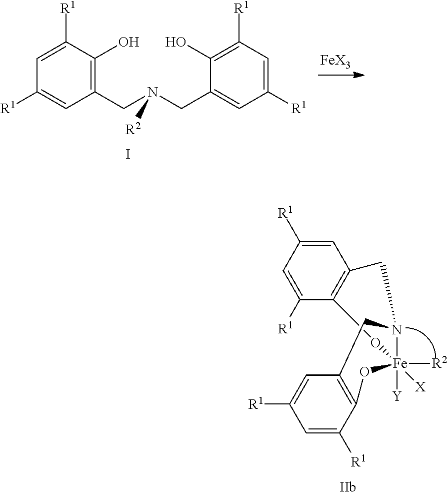

wherein:

R¹, R², X and Y are as defined above in relation to the compound of Formula II; and

wherein R² comprises a coordinating atom coordinated to the Fe atom.

In accordance with another aspect, there is provided a method for cross coupling an alkyl or aryl Grignard reagent with a primary or secondary alkyl halide bearing a β-hydrogen, which comprises reacting the Grignard reagent with the alkyl halide in the presence of an iron complex of Formula II, as defined above, according to the following scheme:

R⁴—MgBr+R⁵—X¹→R⁴—R⁵

wherein:

X¹ is an electronegative atom, such as, for example, Cl, Br and I;

R⁴ is a C₁-C₂₅, or C₁ to C₁₀, substituted or unsubstituted, linear, branched or cyclic alkyl; a substituted or unsubstituted aryl; or a substituted or unsubstituted heterocyclic group; and

R⁵ is a C₁-C₂₅, or C₁ to C₁₀, substituted or unsubstituted, linear, branched or cyclic alkyl, or a C₂-C₂₅ substituted or unsubstituted, linear, branched or cyclic alkenyl or alkynyl; a substituted or unsubstituted aryl or a substituted or unsubstituted heterocyclic group.

In accordance with another aspect, there is provided a method for synthesizing a polymer by controlled radical polymerization, which comprises reacting a monomer and an initiator in the presence of an iron complex having the structure of Formula II

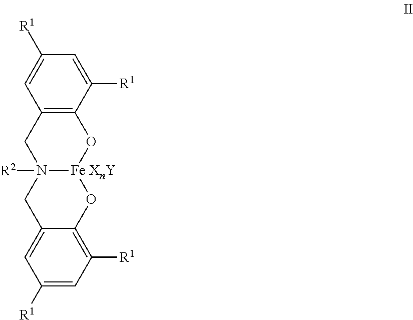

wherein:

each R¹ is independently an electron withdrawing group or a substituted or unsubstituted C₁-C₂₅ linear, branched or cyclic alkyl, or a substituted or unsubstituted aryl;

R² is a substituted or unsubstituted C₁-C₂₅, or C₁ to C₁₀, linear, branched or cyclic alkyl, a substituted or unsubstituted non-aromatic heterocycle, a substituted or unsubstituted aryl, or a substituted or unsubstituted heteroaryl, and optionally comprises a coordinating atom;

X is halogen, such as F, Cl, Br or I;

n is 1 or 2; and

Y is absent or a coordinating solvent molecule, such as water, methanol, ethanol, tetrahydrofuran (THF) or acetonitrile, or, when n is 2, Y is a negatively charged ionic species, such as an alkali metal or NR′″₄⁺, where R′″ is H, alkyl, aryl, heteroalkyl or heteroaryl; wherein when R² comprises a coordinating atom, the coordinating atom forms a dative bond to the Fe atom.

### BRIEF DESCRIPTION OF THE FIGURES

For a better understanding of the present invention, as well as other aspects and further features thereof, reference is made to the following description which is to be used in conjunction with the accompanying drawings, where:

FIGS. 1A and 1B depict the molecular structure (ORTEP) of iron amine-bis(phenolate) compounds {FeCl[O₂N]^{tBuMePr}}₂ and {FeCl[O₂N]^{tBuMeBenzyl}}₂, respectively with partial atom labelling (ellipsoids shown at 50% probability, symmetry operators used to generate equivalent atoms: −x+1, −y+1, −z+1 and hydrogen atoms omitted for clarity);

FIG. 2 depicts the molecular structure (ORTEP) of FeBr₂[O₂]^{tBuMePr} with partial atom labelling (ellipsoids shown at 50% probability, hydrogen atoms omitted for clarity);

FIG. 3 graphically depicts the magnetic moment per mol of iron vs. temperature for {FeCl[O₂N]^{tBuMePr}}₂;

FIG. 4A depicts the molecular structure (ORTEP) and numbering schemes of FeBr[O₂NN′]^{ClClPy}(H₂O) (hydrogen atoms omitted for clarity);

FIG. 4B depicts the molecular structure (ORTEP) of FeBr[O₂NN′]^{ClClPy}(H₂O) showing intermolecular hydrogen bonding (ellipsoids are shown at 50% probability and only the water hydrogen atoms are shown for clarity);

FIG. 5 graphically depicts the UV-vis spectrum of polystyrene LA-216 in CH₂Cl₂ ( [symbol] ) as prepared in Example 5, overlaid on spectrum of CH₂Cl₂ ( [symbol] );

FIG. 6 graphically depicts the Thermogravimetric Analysis (TGA) of polystyrene LA-216 as prepared in Example 5, with T_{d} of 325.81° C.;

FIG. 7 graphically depicts the Differential Scanning Calorimetry (DSC) of polystyrene LA-216 as prepared in Example 5, with T_{g} of 99.47° C.;

FIG. 8 graphically depicts a plot of ln [M]₀/[M]ₜ versus time for bulk styrene polymerization at 120° C. using FeCl[O₂NN′]^{ClClNMe2} and AIBN, with a complex:initiator:monomer ratio of 1:0.6:100.];

FIG. 9 graphically depicts a plot of molecular weight (♦) and PDI () versus conversion for bulk styrene polymerization at 120° C. using FeCl[O₂NN′]^{ClClNMe2} and AIBN, with a complex:initiator:monomer ratio of 1:0.6:100.];

FIG. 10 graphically depicts a stop-start plot of ln [M]₀/[M]ₜ versus time for bulk styrene polymerization at 120° C. using FeCl[O₂NN′]^{ClClNMe2} and AIBN, with a complex:initiator:monomer ratio of 1:0.6:100. PDI values are shown in parentheses;

FIG. 11 graphically depicts the GPC traces for bulk styrene polymerization at 120° C. using FeCl[O₂NN′]^{ClClNMe2} and AIBN, with a complex:monomer ratio of 1:100 (from highest to lowest intensity, the lines depict the reaction speed of reactions having the following equivalents of AIBN: 6 eq, 1.5 eq, 0.6 eq, 0.5 eq, and 0.3 eq);

FIG. 12 depicts the molecular structure (ORTEP) of {FeCl₂[O₂N]^{tBuMePr}}⁻[HN(C₂H₅)₃]⁺ with partial atom labelling (ellipsoids shown at 50% probability, hydrogen atoms omitted for clarity);

FIG. 13 depicts the molecular structure (ORTEP) of FeCl(THF)[O₂N]^{tAmtAmBn} with partial atom labelling (ellipsoids shown at 50% probability, hydrogen atoms omitted for clarity);

FIG. 14 graphically depicts the GPC traces for bulk styrene polymerizations using ^{Cl,Cl,NMe2}[O₂NN′]FeCl as described in Example 7, [Fe][St] ratio 1:100, 120° C., 1 h;

FIGS. 15A and 15B graphically depict the plots of ln([M]₀/[M]ₜ) versus time for bulk styrene polymerizations using 0.8 (), 1.0 (♦) and 2.0 (▪) equivalents of ^{Cl,Cl,NMe2}[O₂NN′]FeCl (15A) and molecular weight versus conversion plots for 0.8 (), 1.0 (♦) and 2.0 (▪) equivalents of ^{Cl,Cl,NMe2}[O₂NN′]FeCl (15B), with dashed line indicating theoretical molecular weights as described in Example 7;

FIG. 16 depicts the molecular structure (ORTEP) of H₂[O₂N]^{BuBuiPr} (H₂L8), H-bonding exists between the hydrogen bond accepter N(1), and the hydrogen donor located on O(2) (H-atoms omitted for clarity (except on atoms O1 and O2), ellipsoids at 50% probability);

FIG. 17 depicts the molecular structure (ORTEP) and partial atom labeling of 10 (ellipsoids shown at 50% probability, and hydrogen atoms omitted for clarity (except at N(2)) along with the co-crystallized toluene molecule;

FIG. 18 depicts the molecular structure (ORTEP) and partial atom labeling of 20 (ellipsoids shown at 50% probability, and hydrogen atoms omitted for clarity);

FIG. 19 depicts the molecular structure (ORTEP) and partial atom labeling of 30 (ellipsoids shown at 50% probability, and hydrogen atoms omitted for clarity);

FIG. 20 depicts the molecular structure (ORTEP) and partial atom labeling of 40 (ellipsoids shown at 30% probability, and hydrogen atoms omitted for clarity (except for H(1)) along with the co-crystallized toluene and pentane molecules;

FIG. 21 depicts the molecular structure (ORTEP) and partial atom labeling of 60 (ellipsoids shown at 30% probability, and hydrogen atoms omitted for clarity (except for H(1)) along with the co-crystallized toluene molecule; and

FIG. 22 graphically depicts the magnetic moment per mol of dimer vs. temperature for complex 50.

### DETAILED DESCRIPTION OF THE INVENTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

As used herein, “halogen” or “halo” refers to F, Cl, Br or I.

As used herein, “alkyl” refers to a linear, branched or cyclic, saturated or unsaturated hydrocarbon group which can be unsubstituted or is optionally substituted with one or more substituent. Examples of saturated straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl and 2-ethyl-1-butyl, 1-heptyl and 1-octyl. As used herein the term “alkyl” encompasses cyclic alkyls, or cycloalkyl groups. The term “cycloalkyl” as used herein refers to a non-aromatic, saturated monocyclic, bicyclic or tricyclic hydrocarbon ring system containing at least 3 carbon atoms. Examples of C₃-C₁₂ cycloalkyl groups include, but are not limited to, cyclopropyl, cyclobutyl, cyclopentyl, cyclohexyl, cycloheptyl, cyclooctyl, norbornyl, adamantyl, bicyclo[2.2.2]oct-2-enyl, and bicyclo[2.2.2]octyl.

As used herein, the term “alkenyl” refers to a straight, branched or cyclic hydrocarbon group containing at least one double bond which can be unsubstituted or optionally substituted with one or more substituents.

As used herein, “alkynyl” refers to an unsaturated, straight or branched chain hydrocarbon group containing at least one triple bond which can be unsubstituted or optionally substituted with one or more substituents.

As used herein, “allenyl” refers to a straight or branched chain hydrocarbon group containing a carbon atom connected by double bonds to two other carbon atoms, which can be unsubstituted or optionally substituted with one or more substituents.

As used herein, “aryl” refers to hydrocarbons derived from benzene or a benzene derivative that are unsaturated aromatic carbocyclic groups of from 6 to 100 carbon atoms, or from which may or may not be a fused ring system, in some embodiments 6 to 50, in other embodiments 6 to 25, and in still other embodiments 6 to 15. The aryls may have a single or multiple rings. The term “aryl” as used herein also includes substituted aryls. Examples include, but are not limited to phenyl, naphthyl, xylene, phenylethane, substituted phenyl, substituted naphthyl, substituted xylene, substituted phenylethane and the like. As used herein, “heteroaryl” refers to an aryl that includes from 1 to 10, in other embodiments 1 to 4, heteroatoms selected from oxygen, nitrogen and sulfur, which can be substituted or unsubstituted.

As used herein, a “heteroatom” refers to an atom that is not carbon or hydrogen, such as nitrogen, oxygen, sulfur, phosphorus, chlorine, bromine, and iodine.

As used herein, a “coordinating atom” refers to an atom having a lone pair of electrons capable of coordinating, or forming a covalent dative bond, with a metal atom.

As used herein, a “heterocycle” is an aromatic or nonaromatic monocyclic or bicyclic ring of carbon atoms and from 1 to 4 heteroatoms selected from oxygen, nitrogen and sulfur, and which can be substituted or unsubstituted. Included within the term “heterocycle” are heteroaryls, as defined above. Examples of 3- to 9-membered heterocycles include, but are not limited to, aziridinyl, oxiranyl, thiiranyl, azirinyl, diaziridinyl, diazirinyl, oxaziridinyl, azetidinyl, azetidinonyl, oxetanyl, thietanyl, piperidinyl, piperazinyl, morpholinyl, pyrrolyl, oxazinyl, thiazinyl, diazinyl, triazinyl, tetrazinyl, imidazolyl, benzimidazolyl, tetrazolyl, indolyl, isoquinolinyl, quinolinyl, quinazolinyl, pyrrolidinyl, purinyl, isoxazolyl, benzisoxazolyl, furanyl, furazanyl, pyridinyl, oxazolyl, benzoxazolyl, thiazolyl, benzthiazolyl, thiophenyl, pyrazolyl, triazolyl, benzodiazolyl, benzotriazolyl, pyrimidinyl, isoindolyl and indazolyl.

As used herein, “substituted” refers to the structure having one or more substituents. A substituent is an atom or group of bonded atoms that can be considered to have replaced one or more hydrogen atoms attached to a parent molecular entity. Examples of substituents include aliphatic groups, halogen, arylcarbonyloxy, alkoxycarbonyloxy, aryloxycarbonyloxy, alkoxycarbonyl, aminocarbonyl, alkylthiocarbonyl, alkoxyl, phosphate ester, phosphonato, phosphinato, cyano, tertiary amino, tertiary acylamino, tertiary amide, imino, alkylthio, arylthio, sulfonato, sulfamoyl, tertiary sulfonamido, nitrile, trifluoromethyl, heterocyclyl, aromatic, and heteroaromatic moieties, ether, ester, boron-containing moieties, tertiary phosphines, and silicon-containing moieties.

As used herein, “olefin”, also called alkene, refers to an unsaturated hydrocarbon containing one or more pairs of carbon atoms linked by a double bond, and includes cyclic or acyclic (aliphatic) olefins, in which the double bond is located between carbon atoms forming part of a cyclic (closed-ring) or of an open-chain grouping, respectively, and monoolefins, diolefins, triolefins, etc., in which the number of double bonds per molecule is, respectively, one, two, three, or some other number. Such olefins can be substituted or unsubstituted. Specific examples of olefins include, but are not limited to, substituted or unsubstituted 1-propene, 1-butene, 1-pentene, 1-hexene, and 1-octene and substituted or unsubstituted norbornene.

As used herein, the term “dative covalent bond” refers to a co-ordinate bond wherein the shared pair of electrons which form the bond come from the same atom. In the present disclosure, the dative covalent bond occurs between the metal, e.g. iron, and the coordinating atom.

As used herein, the terms “ligand” and “amine-bis(phenolate)” refer to the tridentate or tetradentate compound which coordinates iron to form the catalyst. The “amine-bis(phenolate)”refers specifically to a compound having the structure of Formula I, as defined below.

As used herein the terms “catalyst”, “complex” and “amine-bis(phenolate) complex” are used interchangeably to refer to the amine-bis(phenolate) iron complex of Formula II, as defined below.

As used herein, the term “electron withdrawing group” refers to an electronegative group capable of polarizing a bond with a carbon atom. Some non-limiting examples of electron withdrawing groups are halogens, CF₃, nitro, nitrile, carbonyl and substituted carbonyl.

As used herein, the term “initiator” refers to any radical initiator that can produce a radical species to initiate the polymerization reaction. Non-limiting examples of initiators useful in the present polymerization reactions are azo-containing compounds, which have an —N═N— bond. Specific examples of such initiators are azobis(isobutyronitrile) (“AIBN”), V-65 and V-70.

Catalysts described herein are provided with abbreviated notation for clarity and simplicity. For the iron tridentate amine-bis(phenolate) complexes of structure IIa

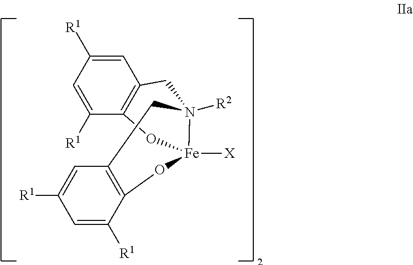

the simplified notation is {FeX[O₂N]^{R1(ortho)R1(para)R2}}₂. For example, in the case that both R¹ groups ortho to the respective phenolates are butyl, and both R¹ groups para to the respective phenolates are methyl, X is Cl and R² is propyl, the simplified notation is {FeCl[O₂N]^{BuMePr}}₂.

For tetratendate iron amine bis(phenolate) complexes having the following structure,

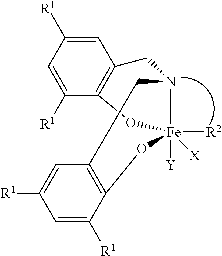

the simplified notation is FeX[O₂N(coordinating atom)′]^{R1(ortho)R1(para)R2}. For example, in the case that both R¹ groups ortho to the respective phenolates are butyl, both R¹ groups para to the respective phenolates are methyl, X is Cl, Y is absent and R² is pyridinyl, the simplified notation is FeCl[O₂NN′]^{BuMePy}. When Y is OH₂, the complex has the simplified notation FeCl[O₂NN′]^{BuMePy}(H₂O).

As would be recognized by a worker skilled in the art, it is not necessary for both R¹ groups ortho to the respective phenolates to be the same or for both R¹ groups para to the respective phenolates. The R¹ groups ortho to the respective phenolates can be the same or different. Similarly, the R¹ groups para to the respective phenolates can be the same or different.

Amine-bis(phenolate) Ligands and Iron Complexes

Described herein are amine (bisphenolate) ligands which, when coordinated with iron are useful as catalysts in, for example, carbon cross-coupling in Grignard reactions, and controlled radical polymerization reactions. These iron bisphenolate complexes can catalyze carbon cross coupling in alkyl and aryl Grignard reactions with alkyl halides. These iron bisphenolate complexes can also catalyze polymerization of various monomers to provide polymers with low polydispersity indices. In certain examples, these polymers are also substantially free from coloured contaminants.

The amine-bis(phenolate) ligands have the structure of Formula I:

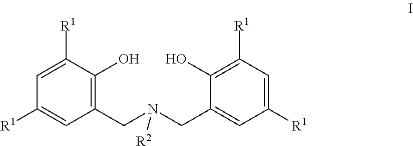

wherein

each R¹ is independently an electron withdrawing group; and

R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom.

with the proviso that when the R¹ substituents are all Cl, R² cannot be CH₂, CH₂CH₂N(CH₃)₂, or CH₂CH₂OCH₃, and when the R¹ substituents are all Br, R² cannot be CH₃ or CH₂CH₂N(CH₃)₂.

The coordinating atom can be, for example, any Group 15 element (nitrogen, phosphorus, arsenic, antimony and bismuth) or Group 16 element (oxygen, sulfur, selenium, or tellurium) or a carbenic atom of a carbene-containing fragment (such as an N-heterocyclic carbene). In a specific embodiment, the coordinating atom is an aprotic N or O atom.

The ligand can be tridentate, wherein the pendant group R² does not comprise a coordinating moiety. Such ligands, when bound to a metal, have three loci to form dative covalent bonds, specifically one dative covalent bond can form from each phenolate, and a third can form from the central nitrogen. Some preferred examples of ligands wherein the pendant group R² does not comprise a coordinating moiety, include those ligands having an R² group that is a C₁₋₆ straight, branched or cyclic alkyl group.

Specific, non-limiting, examples of tridentate amine (bisphenolate) ligands useful in the formation of iron complexes of Formula IIa are listed below:

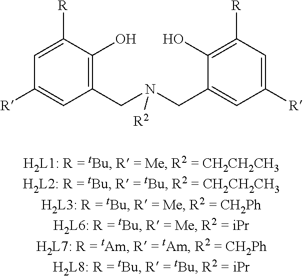

The ligand can also be tetradentate when the pendant R² group comprises a coordinating atom. In a specific example, the coordinating atom is an aprotic heteroatom, such as oxygen or nitrogen, in a heterocycle, heteroaryl or a substituted alkyl moiety. In accordance with one embodiment, the coordinating atom is a heteroatom incorporated into a C₁₋₆ straight, branched or cyclic alkyl group. In accordance with another embodiment, the coordinating atom is a heteroatom in a heterocycle, such as, for example, pyridyl, furanyl, furfural or tetrahydrofuranyl.

Specific, non-limiting, examples of tetradentate amine (bisphenolate) ligands useful in the formation of iron complexes of Formula IIb are listed below:

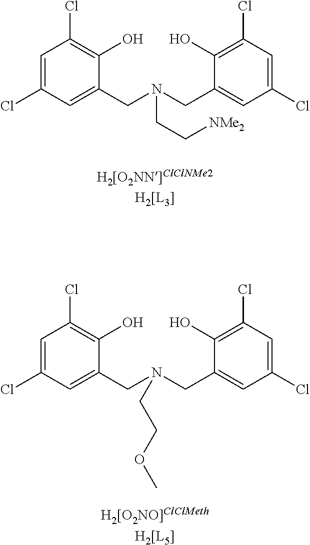 [SMILES: CN(C)CCN(Cc1cc(Cl)cc(Cl)c1O)Cc1cc(Cl)cc(Cl)c1O ; COCCN(Cc1cc(Cl)cc(Cl)c1O)Cc1cc(Cl)cc(Cl)c1O]

Specific, non-limiting, examples of tetradentate amine (bisphenolate) ligands of Formula I, which are useful in the formation of iron complexes of Formula IIb, are listed below:

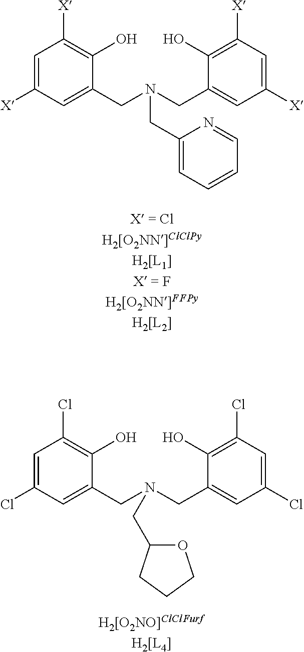

The present amine-bis(phenolate) ligands are readily synthesized by a modified Mannich condensation in water, for example, as shown in Scheme 2.

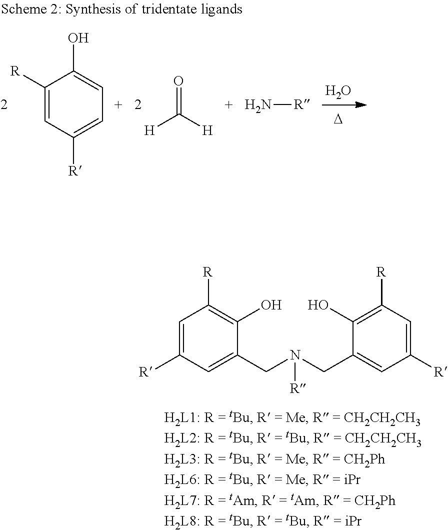

In accordance with another aspect, there is provided an iron amine-bis(phenolate)complex having the structure of Formula II:

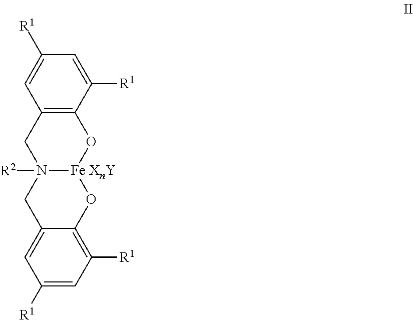

wherein:

each R¹ is independently an electron withdrawing group or a substituted or unsubstituted C₁-C₂₅ linear, branched or cyclic alkyl, or a substituted or unsubstituted aryl, where R¹ does not comprise a coordinating atom

R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom;

X is halogen, such as F, Cl, Br or I;

n is 1 or 2; and

Y is absent or a coordinating solvent molecule, such as water, methanol, ethanol, tetrahydrofuran (THF) or acetonitrile, or, when n is 2, Y is a positively charged ionic species, such as an alkali metal or NR′″₄⁺, where R′″ is H, alkyl, aryl, heteroalkyl or heteroaryl; wherein when R² comprises a coordinating atom, the coordinating atom forms a dative bond to the Fe atom, and with the proviso that when the R¹ substituents are all methyl, R² cannot be CH₂CH₂N(CH₃)₂, when the two ortho R¹ substituents are tert-butyl and the two para R¹ substituents are methyl, R² cannot be —CH₂pyridine or CH₂CH₂N(CH₃)₂, and when R² is —CH₂(2-tetrahydrofuran) or —CH₂CH₂methoxy, R¹ is an electron withdrawing group.

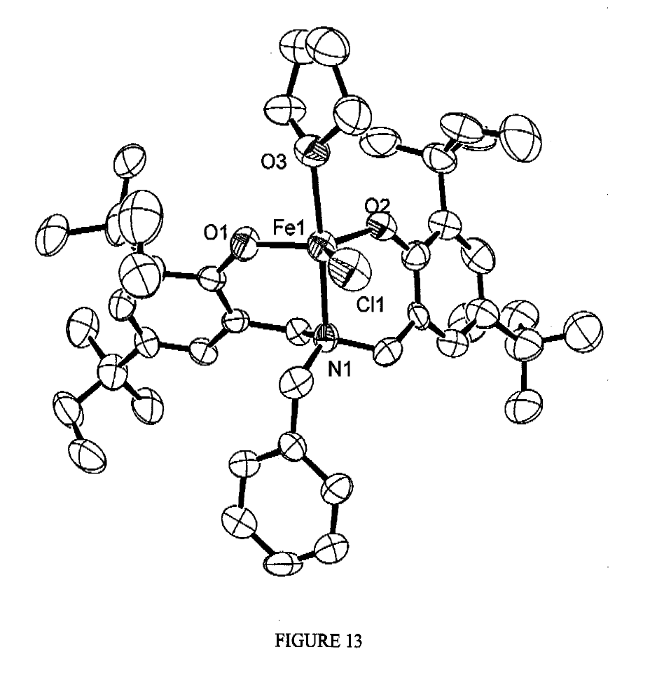

As noted above, in certain examples, the iron amine-bis(phenolate) complex comprises two coordinated halogen atoms X. In these examples, the complex is a negatively charged species associated with a positively charged ionic species Y. The positively charged ionic species can be any atom or molecule that can function as a positive counter ion to balance the negative charge of the complex. FIG. 12 depicts a crystal structure of an iron complex of a tridentate ligand formulated as {FeCl₂[O₂N]^{tBuMePr}}⁻[HN(C₂H₅)₃]⁺.

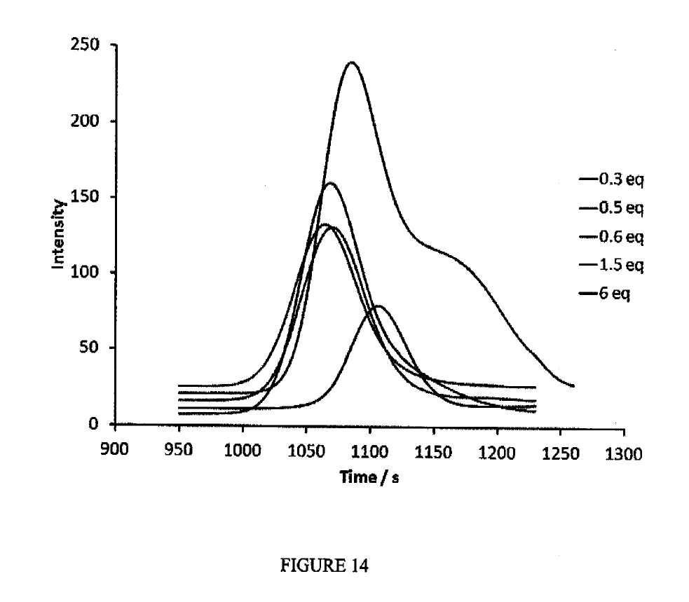

Furthermore, certain examples of the iron amine-bis(phenolate) complex comprise a coordinated solvent molecule Y. The nature of the solvent molecule will depend on the solvents used in the preparation and purification of the complex. Non-limiting examples of coordinated solvent molecules are water, methanol, ethanol, tetrahydrofuran (THF) and acetonitrile. FIG. 13 depicts a crystal structure of an iron complex of a tridentate ligand comprising coordinated THF (FeCl(THF)[O₂N]^{tAmtAmBa}).

In accordance with one embodiment of the compound of Formula II, there is provided an iron complex having the structure of Formula IIa or IIa′:

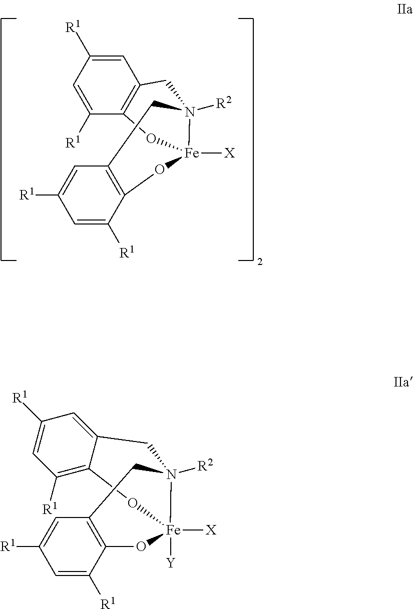

wherein:

R¹, R², X and Y are as defined above in relation to the compound of Formula II; and

wherein R² does not comprise a coordinating atom bound via a covalent dative bond to the Fe atom.

The dimeric form of iron amine-bis(phenolate) complex, i.e. Formula IIa, only exists in the solid state. As soon as it is dissolved, for example, for use as a catalyst, the complex separates to form the monomer complex of Formula IIa′. In a specific example, when the complex is dissolved, it coordinates a solvent molecule Y as an additional ligand.

In accordance with another embodiment of the compound of Formula II, there is provided an iron complex having the structure of Formula IIb:

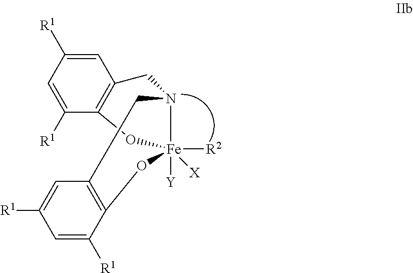

wherein:

R¹, R², X and Y are as defined above in relation to the compound of Formula II; and wherein R² comprises a coordinating atom bound via a covalent dative bond to the Fe atom.

In the synthesis of an iron amine-bis(phenolate) complex of Formula II, a tridentate ligand can be reacted with iron or an iron salt to form the iron amine-bis(phenolate) complexes. Accordingly, also provided herein is a method for synthesizing iron amine-bis(phenolate) complexes from tridentate ligands, for example, as set out in Scheme 3 below.

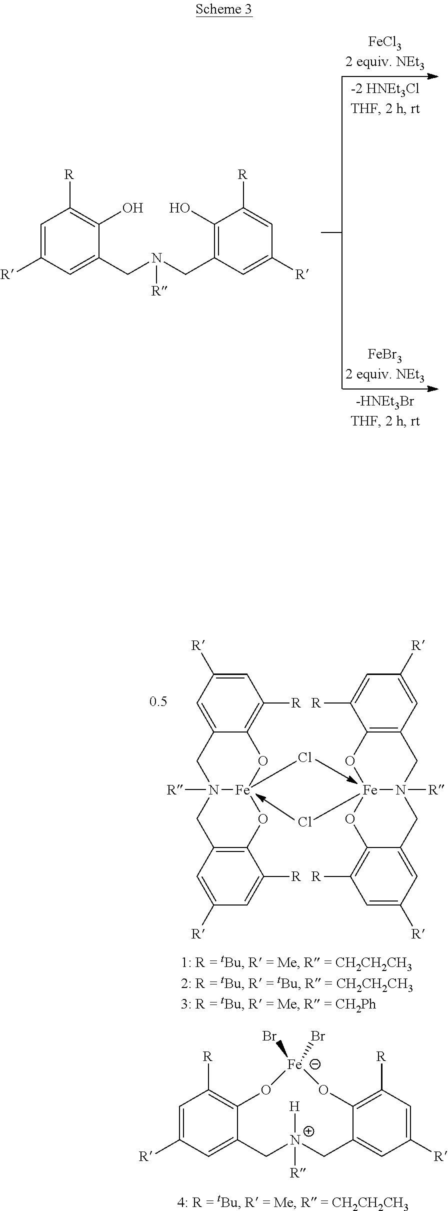

The amine-bis(phenolate) ligand precursors, abbreviated H₂[O₂N]^{RR′R″} (where R=^{t}Bu, R′=Me, R″=n-propyl (H₂L1); R=R¹=^{t}Bu, R″=n-propyl (H₂L2) and R=^{t}Bu, R′=Me, R″=benzyl (H₂L3)) were prepared by modification of literature procedures, (Kerton et al. 2008 Can. J. Chem. Vol. 86 p. 435; Tshuva et al. 2001 Organometallics Vol. 20, p. 3017; Chmura et al 2006 Dalton Transactions p. 887) and as shown above.

The ligands H₂L1, H₂L2 and H₂L3 shown in Scheme 3 above react with FeCl₃ in THF in the presence of base to generate immediate colour changes to dark purple. From these solutions, the chloride-bridged dimeric iron(III) complexes ([FeL1(μ-Cl)]₂ ({FeCl[O₂N]^{tBuMePr}}), [FeL2(μ-Cl)]₂ ({FeCl[O₂N]^{tBuBuPr}}) and [FeL3(μ-Cl)]₂ ({FeCl[O₂N]^{tBuMeBenzyl}}) were isolated in high yield.

There is also provided herein a method for synthesizing iron amine-bis(phenolate) complexes from tetradentate ligands according to the following reaction:

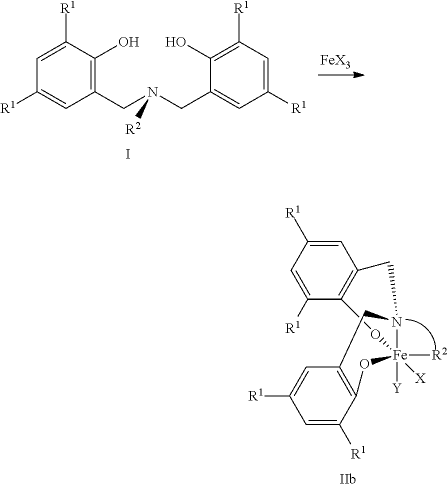

In the tetradentate ligand complexes of this type, the R² pendant group comprises a coordinating atom that can form a dative covalent bond with the Fe atom.

The amine-bis(phenolate) iron complexes described herein have been found to be robust, stable towards water and oxygen, and are readily synthesized from inexpensive starting materials. Accordingly, also provided herein is a catalyst system comprising a catalyst of Formula II and, optionally, one or more solvents or reactants, such as an initiator. As would be readily appreciated by a worker skilled in the art, selection of the appropriate solvent(s) and/or reactants will depend on the reaction being catalyzed and the required reaction conditions.

Application of Amine-bis(phenolate) Iron Complexes

Carbon Cross-Coupling

The amine-bis(phenolate) iron complexes described herein are useful in catalytic cross-coupling of alkyl and aryl Grignard reagents with a primary or secondary alkyl halide bearing a β-hydrogen. The cross-coupling reaction proceeds by reacting the Grignard reagent with the alkyl halide, in the presence of an amine-bis(phenolate) catalyst of Formula II, according to the following equation:

R⁴—MgBr+R⁵—X¹→R⁴—R⁵

wherein:

X¹ is an electronegative atom, such as, for example, Cl, Br or I;

R₄ is a C₁-C₂₅ substituted or unsubstituted, linear, branched or cyclic alkyl; a substituted or unsubstituted aryl; or a substituted or unsubstituted heterocyclic group;

R₅ is a C₁-C₂₅ substituted or unsubstituted, linear, branched or cyclic alkyl, or a C₂-C₂₅ substituted or unsubstituted, linear, branched or cyclic alkenyl or alkynyl; a substituted or unsubstituted aryl or a substituted or unsubstituted heterocyclic group.

The reaction can be conducted at room temperature or under heating. It has been found that improved yields can be obtained for certain substrates by performing the reaction under microwave heating. Preferably, the amine-bis(phenolate) catalysts used in the cross-coupling reactions do not include R¹ and R² substituents that are substituted with F.

Controlled Radical Polymerization

Also provided herein is a method of using the amine-bis(phenolate) iron complexes as catalysts for atom transfer radical polymerization (ATRP). ATRP is an example of a controlled radical polymerization (CRP) whereby radical concentrations are kept low by a rapid and reversible trapping by a metal-halogen species. The growing polymer chain is capped by a halogen, in this case chlorine or bromine. The dormant chain can react with a low oxidation state transition metal, forming a growing free radical and a new metal-halogen bond. The present system is a reverse ATRP protocol whereby a radical initiator and a high oxidation state metal halide cooperate to control the radical reactivity.

Traditional complexes for ATRP depend upon copper and ruthenium metal mediators. Use of both of these catalyst systems in ARTP results in polymers that retain highly coloured transition metals. In contrast, the presently described amine-bis(phenolate) iron complex systems produce bright white polymeric materials upon polymer precipitation. The rapid rates, high levels of control and ease of synthesis of these complexes contribute to the strength of this catalyst system. In addition, iron is an environmentally and biochemically benign metal, which means that these catalyst systems can be useful in food and biomedical applications.

The present amine-bis(phenolate) iron complexes can capably control the polymerization of various monomers. The monomer can include, but are not limited to, acrylates, methacrylates, styrenes, acrylonitriles, vinyl acetate, vinyl pyrrolidones, and combinations thereof. Preferred monomers are styrene, methyl methacrylate, methyl acrylate, vinyl acetate, and combinations thereof.

The rates of polymerization of these monomers using the present catalyst systems are comparable to those observed for copper complexes, but the polymers produced are white and the remaining iron is benign. Extensive screening and kinetic plots to confirm the complexes activity as ATRP mediators are provided in the Examples below.

There is also provided a method to prepare low dispersity polymers (i.e., those having a low polydispersity index (“PDI”)) of controlled molecular weight through a radical mechanism process using the amine-bis(phenolate) iron complexes. For reference, a method having “good control” is considered to be one that produces polymers having a PDI of <1.4. The best iron complexes reported in the literature can reach a PDI of 1.2. Levels of control attainable using the present amine-bis(phenolate) iron catalyst systems are excellent, as demonstrated by the fact that PDIs of down to 1.10 have been achieved from ATRP of styrene and down to 1.16 from ATRP of methyl methacrylate. The present catalysts and methods can thus provide rapid production of white polystyrenes and polyacrylates of predictable molecular weight and low polydispersity.

The choice of catalyst and monomer are made together, matching the Fe—Cl bond strength to that of the capped polymer chain C—Cl bond strength. In general, this means a singly or doubly substituted double bond with at least one R group containing an activating functionality. These functionalities are either aryl groups or esters but can be expanded to anything that will induce polarity in the double bond and stabilize the radical at the alpha carbon.

Preferably, the amine-bis(phenolate) catalysts used in the ARTP reactions do not include R¹ and R² alkyl substituents that are substituted with higher halogens (Cl, Br and I) or R¹ and R² aryl substituents that are substituted with I.

To gain a better understanding of the invention described herein, the following example is set forth. It should be understood that these examples are for illustrative purposes only. Therefore, they should not limit the scope of this invention in any way.

### EXAMPLES

### Example 1

### Preparation and characterization of Tridentate Ligands and Complexes

Synthesis of Iron Complex {FeCl[O₂N]^{tBuMePr}}₂

Synthesis of Ligand:

To a stirred mixture of 2-t-butyl-4-methylphenol (20,236 g, 0.1232 mol) in 75 mL deionized water was added 37% aqueous formaldehyde (10 mL, 0.1232 mol) followed by slow addition of n-propyl amine (3.64 g, 0.0615 mol). The reaction was heated to reflux for 12 h. Upon cooling, the reaction mixture separated into two phases. The upper phase was decanted and the remaining oily residue was triturated with cold methanol to give an analytically pure, white powder (23.01 g, 91%). Anal. Calcd for C₂₇H₄₁NO₂: C, 78.78; H, 10.04; N, 3.40. Found C, 78.85; H, 10.14; N, 3.32. ¹H NMR (500 MHz, CDCl₃, δ): 7.00 (d, J=1.6 Hz, ArH, 2H); 6.73 (d, J=1.6 Hz, ArH, 2H); 3.63 (s, CH₂, 4H); 2.48 (t, J=7.5 Hz, CH₂, 2H); 2.24 (s, CH₃, 6H); 1.62 (m, CH₂, 2H); 1.39 (s, CH₃, 18H); 0.86 (t, J=7.5 Hz, CH₃, 3H). ¹³C{¹H}NMR (125 MHz, 298 K, CDCl₃): δ 152.7 (Ar); 137.0 (Ar); 129.1 (Ar); 128.3 (Ar); 127.5 (Ar); 122.47 (Ar); 122.7 (Ar); 57.3 (CH₂); 55.7 (CH₂); 34.8 (C(CH₃)₃); 29.9 (C(CH₃)₃); 21.0 (ArCH₃); 19.7 (CH₂); 12.0 (CH₃).

Synthesis of Iron Complex:

To an ethanolic slurry of recrystallized ligand (2.68 g, 6.52 mmol) was added a solution of anhydrous FeCl₃ (1.06 g, 6.52 mmol) in THF resulting in an intense purple solution. To this solution was added triethylamine (1.32 g, 13.04 mmol) and the resulting mixture was stirred for 2 h then filtered through Celite. Removal of solvent under vacuum yielded a dark purple product (2.87 g, 88%). Crystals suitable for X-ray diffraction were obtained by slow evaporation of a toluene solution (2.054 g, 63%). Anal. Calcd for C₂₇H₃₉ClFeNO₂: C, 64.74; H, 8.11; N, 2.99. Found: C, 65.02; H, 7.85; N, 2.80. MS (MALDI-TOF) m/z (%, ion): 408.1 (26, [M-FeCl]⁺), 464.2 (100, [M-Cl]⁺), 499.2 (20, [M]⁺), 965.45 (10, [2M-Cl]⁺). UV-vis (solvent) λₘₐₓ, nm (ε): (Pentane) 480 (2800), 320 (2800); (MeCN) 500 (2580), 320 (2740), 240 (5500); (MeOH) 600 (2120), 330 (2260), 250 (5170); (THF) 490 (2800), 320 (2930), 250 (5880). μ_{eff} (solid, 27° C.) 5.50 μ_{B} per mol Fe.

Synthesis of Iron Complex {FeCl[O₂N]^{tButBuPr}}₂:

Synthesis of Ligand:

To a stirred mixture of 2,4-di-t-butylphenol (20.236 g, 0.1232 mol) in 75 mL deionized water was added 37% aqueous formaldehyde (10 mL, 0.1232 mol) followed by slow addition of n-propyl amine (3.64 g, 0.0615 mol). The reaction was heated to reflux for 12 h. Upon cooling, the reaction mixture separated into two phases. The upper phase was decanted and the remaining oily residue was triturated with cold methanol to give a pure, white powder (23.01 g, 91%). Spectroscopic analyses by NMR are consistent with those previously reported therefore no elemental analysis is reported here.^{24 1}H NMR (300 MHz, CDCl₃, δ): 7.25 (s, ArH, 2H); 6.95 (s, ArH, 2H); 3.70 (s, CH₂, 4H); 2.53 (t, ³J=7.5 Hz, CH, 2H); 1.65 (m, CH₂, 2H); 1.42 (s, CH₃, 18H); 1.30 (s, CH₃, 18H); 0.90 (t, ³J=7.5 Hz, CH₃, 3H). ¹³C{¹H}NMR (75 MHz, 298 K, CDCl₃): δ 152.40 (Ar); 141.5 (Ar) 136.04 (Ar); 128.93 (Ar); 128.08 (Ar); 125.06 (Ar); 123.48 (Ar); 121.76 (Ar); 57.24 (ArCH₂); 55.53 (ArCH₂); 34.88 (C(CH₃)₃); 34.22 (C(CH₃)₃); 31.68 (C(CH₃)₃); 29.73 (C(CH₃)₃); 19.40 (CH₂); 16.67 (CH(CH₃)₂).

Synthesis of Iron Complex:

To an ethanolic slurry of recrystallized ligand (3.23 g, 6.52 mmol) was added a solution of anhydrous FeCl₃ (1.06 g, 6.52 mmol) in THF resulting in an intense purple solution. To this solution was added triethylamine (1.32 g, 13.04 mmol) and the resulting mixture was stirred for 2 h then filtered through Celite. Removal of solvent under vacuum yielded a dark purple product (3.43 g, 90%). Crystals suitable for X-ray diffraction were obtained by slow evaporation of a toluene solution (2.29 g, 60%). Anal. Calcd for C₃₃H₅₁ClFeNO₂: C, 67.75; H, 8.79; N, 2.39. Found: C, 67.89; H, 8.85; N, 2.50. MS (MALDI-TOF) m/z (%, ion): 495.4 (25, [M-FeCl]⁺), 548.3 (100, [M-Cl]⁺), 584.3 (20, [M]⁺). UV-vis (solvent) λₘₐₓ, nm (ε): (Pentane) 480 (2800), 330 (2700); (MeCN) 500 (2600), 330 (2700), 250 (5570); (MeOH) 600 (2100), 320 (2250), 250 (5200); (THF) 500 (2850), 320 (3000), 250 (5900). μ_{eff} (solution, 25° C.) 5.6 μ_{B} per mol Fe.

Synthesis of Iron Complex {FeCl[O₂N]^{tBuMeBenzyl}}₂

Synthesis of Ligand:

To a stirred mixture of 2-t-butyl-4-methylphenol (20.236 g, 0.1232 mol) in 75 mL deionized water was added 37% aqueous formaldehyde (10 mL, 0.1232 mol) followed by slow addition of benzylamine (3.64 g, 0.0615 mol). The reaction was heated to reflux for 12 h. Upon cooling, the reaction mixture separated into two phases. The upper phase was decanted and the remaining oily residue was triturated with cold methanol to give an analytically pure, white powder (23.01 g, 91%). Anal. Calcd for C₃₁H₄₁NO₂: C, 81.00; H, 8.99; N, 3.05. Found C, 79.83; H, 9.10; N, 3.00. ¹H NMR (300 MHz, CDCl₃, δ): 7.37 (s, ArH, 1H); 7.35 (s, ArH, 1H); 7.32 (s, ArH, 1H); 7.29 (s, ArH, 1H); 7.22 (s, ArH, 1H); 6.99 (d, J=1.6 Hz, ArH, 2H); 6.74 (d, J=1.6 Hz, ArH, 2H); 3.58 (s, CH₂, 4H); 3.53 (s, CH₂, 2H); 2.22 (s, CH₃, 6H); 1.38 (s, CH₃, 18H). ¹³C{¹H}NMR (75 MHz, 298 K, CDCl₃): δ 152.27 (Ar); 137.59 (Ar); 136.76 (Ar); 129.56 (Ar); 129.06 (Ar); 129.02 (Ar); 128.04 (Ar); 127.99 (Ar); 127.44 (Ar); 58.51 (CH₂); 56.59 (CH₂); 34.66 (C(CH₃)₃); 29.62 (C(CH₃)₃); 20.81 (ArCH₃),

Synthesis of Iron Complex:

To an ethanolic slurry of recrystallized ligand (3.00 g, 6.52 mmol) was added a solution of anhydrous FeCl₃ (1.06 g, 6.52 mmol) in THF resulting in an intense purple solution. To this solution was added triethylamine (1.32 g, 13.04 mmol) and the resulting mixture was stirred for 2 h then filtered through Celite. Removal of solvent under vacuum yielded a dark purple product (2.86 g, 80%). Crystals suitable for X-ray diffraction were obtained by slow evaporation of a toluene solution (2.15 g, 60%). Anal. Calcd for C₃₁H₃₉ClFeNO₂: C, 67.83; H, 7.16; N, 2.55. Found: C, 68.02; H, 7.25; N, 2.63. MS (MALDI-TOF) m/z (%, ion): 457.3 (20, [M-FeCl]⁺), 513.2 (100, [M-Cl]⁺), 548.2 (20, [M]⁺). UV-vis (solvent) λₘₐₓ, nm (ε): (Pentane) 490 (1120), 320 (1300); (MeCN) 500 (2010), 350 (1350), 230 (4750); (MeOH) 520 (1000), 320 (1800), 250 (5070); (THF) 500 (1890), 320 (2800), 250 (4880). μ_{eff} (solution, 25° C.) 5.5 μ_{B} per mol Fe.

Synthesis of Iron Complex FeBr₂[O₂]^{tBuMePr}:

The ligand was prepared according to the method provided in the synthesis of iron complex {FeCl[O₂N]^{tBuMePr}}₂ described above.

Synthesis of Iron Complex:

To an ethanolic slurry of recrystallized ligand (2.68 g, 6.52 mmol) was added a solution of anhydrous FeBr₃ (1.93 g, 6.52 mmol) in THF resulting in an intense purple solution. To this solution was added triethylamine (1.32 g, 13.04 mmol) and the resulting mixture was stirred for 2 h then filtered through Celite. Removal of solvent under vacuum yielded a dark purple product (3.06 g, 75%). Crystals suitable for X-ray diffraction were obtained by slow evaporation of a toluene solution (2.04 g, 50%). Anal. Calcd for C₂₇H₄₀Br₂FeNO₂: C, 51.78; H, 6.44; N, 2.24. Found: C, 52.02; H, 6.54; N, 2.57. MS (MALDI-TOF) m/z (%, ion): 465.2 (100, [M-2Br]⁺), 545.2 (48, [M-Br]⁺), 626.4 (10, [M]⁺). UV-vis (solvent) λₘₐₓ, nm (ε): (Pentane) 500 (1240), 340 (1700); (MeCN) 490 (1270), 340 (1560); (MeOH) 600 (940), 330 (1670); (THF) 490 (2030), 330 (2330). μ_{eff} (solution, 25° C.) 5.8 μ_{B} per mol Fe.

Structural and Electronic Characterization of Iron Complexes

The ¹H NMR spectra exhibit broad, shifted peaks consistent with paramagnetism. Single crystals of {FeCl[O₂N]^{tBuMePr}}₂, {FeCl[O₂N]^{tBuMeCBenzyl}}₂ and FeBr₂[O₂]^{tBuMePr} suitable for X-ray diffraction were obtained by slow evaporation of toluene solutions. Suitable crystals were selected and mounted on a diffraction loop using Paratone-N oil and cooling to 153 K or lower. Complete crystallographic data, bond lengths and bond angles are given in supporting information.

All measurements were made on a Rigaku Saturn CCD area detector with graphite monochromated Mo-Kα radiation. The data was processed and corrected for Lorentz and polarization effects and absorption. Neutral atom scattering factors for all non-hydrogen atoms were taken from the International Tables for X-ray Crystallography. The structure was solved by direct methods and expanded using Fourier techniques. All non-hydrogen atoms were refined anisotropically. Hydrogen atoms were refined using the riding model. Anomalous dispersion effects were included in F_{calc}; the values for Δf′ and Δf″ were those of Creagh and McAuley. (International Tables for Crystallography, ed. A. J. C. Wilson, Kluwer Academic Publishers, Boston, 1992, 219-222.) The values for the mass attenuation coefficients are those of Creagh and Hubbell. (International Tables for Crystallography, ed. A. J. C. Wilson, Kluwer Academic Publishers, Boston, 1992, 200-206.) All calculations were performed using the CrystalStructure⁴¹ crystallographic software package except for refinement, which was performed using SHELXL-97. Crystallographic data are given in Table 1,

TABLE 1
Selected bond lengths (Å) and angles (°) of {FeCl[O₂N]^{tBuMePr}}₂, {FeCl[O₂N]^{tBuMeBenzyl}}₂ and FeBr₂[O₂]^{tBuMePr}

| | {FeCl[O₂N]^{tBuMePr}}₂ | {FeCl[O₂N]^{tBuMeBenzyl}}₂ | FeBr₂[O₂]^{tBuMePr} |
|---|---|---|---|
| Fe(1)-O(1) | 1.818(3) | 1.8276(12) | 1.828(3) |
| Fe(1)-O(2) | 1.817(3) | 1.8222(12) | 1.836(3) |
| Fe(1)-N(1) | 2.183(4) | 2.1818(13) | |
| Fe(1)-N(6) | | | 3.435(3) |
| Fe(1)-Cl(1) | 2.298(2) | 2.3290(5) | |
| Fe(1)-Cl(1)* | 2.4911(18) | | |
| Fe(1)-Cl(2) | | 2.5026(5) | |
| Fe(1)-Br(1) | | | 2.3569(7) |
| Fe(1)-Br(2) | | | 2.3723(7) |
| Fe•••Fe | 3.4658(7) | 3.5748(3) | |
| O(1)-Fe(1)-O(2) | 124.63(14) | 119.36(6) | 105.23(15) |
| N(1)-Fe(1)-Cl(1)* | 178.32(9) | | |
| N(1)-Fe(1)-Cl(2) | | 177.29(4) | |
| Cl(1)-Fe(1)-Cl(1)* | 87.36(6) | | |
| Cl(1)-Fe(1)-Cl(2) | | 84.343(19) | |
| Br(1)-Fe(1)-Br(2) | | | 109.55(3) |
| O(1)-Fe(1)-Br(1) | | | 110.72(10) |
| O(2)-Fe(1)-Br(1) | | | 112.88(11) |
| O(1)-Fe(1)-Br(2) | | | 109.41(10) |
| O(2)-Fe(1)-Br(2) | | | 108.93(11) |
| Fe(1)-Cl(1)-Fe(1)* | 92.64(6) | | |
| Fe(1)-Cl(1)-Fe(2) | | 95.38(2) | |
| O(1)-Fe(1)-Cl(1) | 122.08(12) | 114.96(4) | |
| O(1)-Fe(1)-Cl(1)* | 89.41(11) | | |
| O(1)-Fe(1)-Cl(2) | | 88.91(4) | |
| O(2)-Fe(1)-Cl(1) | 113.18(11) | 125.52(4) | |
| O(2)-Fe(1)-Cl(1)* | 89.86(11) | | |
| O(2)-Fe(1)-Cl(2) | | 92.60(4) | |
| O(1)-Fe(1)-N(1) | 88.99(13) | 90.38(5) | |
| O(2)-Fe(1)-N(1) | 90.62(13) | 90.03(5) | |

*Symmetry operators used to generate equivalent atoms: −x + 1, −y + 1, −z + 1.

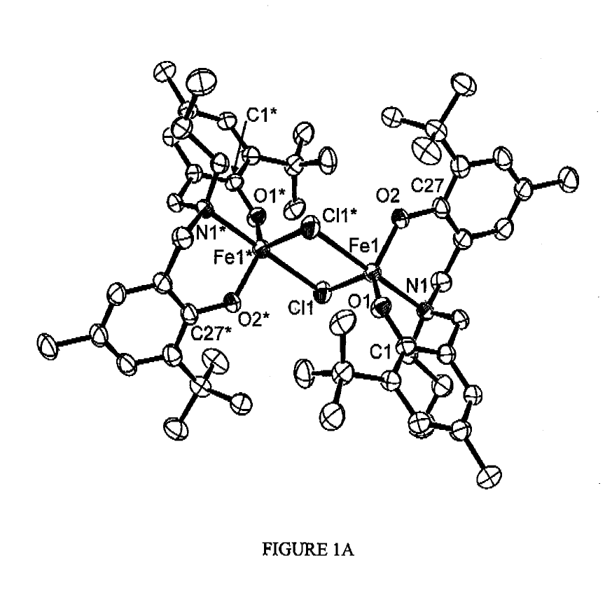

In the solid state, iron complexes {FeCl[O₂N]^{tBuMePr}}₂, {FeCl[O₂N]^{tButBuPr}}₂, and {FeCl[O₂N]^{tBuMeBenzyl}}₂ exhibit dimeric structures resulting in trigonal bipyramidal iron(III) centres bridged by chloride ligands as shown in FIGS. 1A and 1B. The Fe(1) . . . Fe(1)* distance of 3.4658(7) Å in {FeCl[O₂N]^{tBuMePr}}₂ and Fe(1) . . . Fe(2) distance of 3.5748(3) Å in {FeCl[₂N]^{tBuMeBenzyl}}₂ precludes any bonding interaction between the metal centres. The two phenolate oxygen donor atoms and a bridging chloride occupy the equatorial plane around each iron ion, where the sum of bond angles is 359.89° in {FeCl[O₂N]^{tBuMePr}}₂ and 359.84° in {FeCl[O₂N]^{tBuMeBenzyl}}₂ indicating near perfect planarity. The amine nitrogen and another bridging chloride take up the axial positions, giving a Cl(1)-Fe(1)-N(1) bond angle of 178.32(9)° in {FeCl[O₂N]^{tBuMeBenzyl}}₂ and Cl(2)-Fe(1)-N(1) bond angle of 177.28(3)° in {FeCl[O₂N]^{tBuMeBenzyl}}₂. The cis-orientated chloride ligands are nearly orthogonal with a Cl—Fe—Cl bond angle of 87.36(4)° in {FeCl[O₂N]^{tBuMePr}}₂ and 84.341(14)° in {FeCl[O₂N]^{tBuMeBenzyl}}₂. The asymmetric nature of the bridging chlorides is demonstrated by the different Fe—Cl bond lengths of 2.2976(12) Å for Fe(1)-Cl(1) and 2.4912(14) Å for Fe(1)-Cl(1)* in {FeCl[O₂N]^{tBuMePr}}₂ and 2.3290(4) Å for Fe(1)-Cl(1) and 2.5025(3) Å for Fe(1)-Cl(2) in {FeCl[O₂N]^{tBuMeBenzyl}}₂.

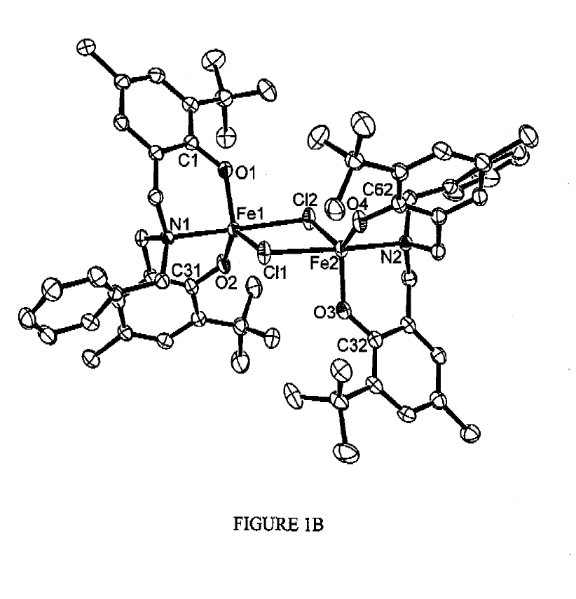

Single crystal X-ray diffraction on compound FeBr₂[O₂]^{tBuMePr} showed its solid state structure to be different to that of the other three prepared iron complexes. The molecular structure of FeBr₂[O₂]^{tBuMePr} is shown in FIG. 2. In the solid state, complex FeBr₂[O₂]^{tBuMePr} exhibits a monomeric structure having a tetrahedral iron(II) centre. Unlike the other three complexes, the bis(phenolate) ligand in FeBr₂[O₂]^{tBuMePr} binds in a bidentate fashion. The central amine nitrogen atom is protonated resulting in a quaternized ammonium group. The two phenolate groups remain anionic, thereby resulting in a net monoanionic ammonium-bis(phenolate) ligand. The iron(III) centre is further bonded to two bromide ions, hence the four-coordinate iron(III) centre is formally anionic, resulting in an overall zwitterionic complex. The two phenolate oxygen donor atoms and two bromide ions compose the tetrahedral ligand sphere around iron. The bond angles around the metal ranged from 105.23(15)° to 112.88(11)°, which are only moderately distorted from the ideal tetrahedral angle of 109.5°. The bond lengths of Fe—Br(1) and Fe—Br(2) are slightly asymmetrical at 2.3569(7) and 2.3723(7) Å respectively. The phenolate oxygen atoms exhibited bond distances to iron of 1.828(3) and 1.836(3) Å for Fe(1)-O(1) and Fe(1)-O(2), respectively.

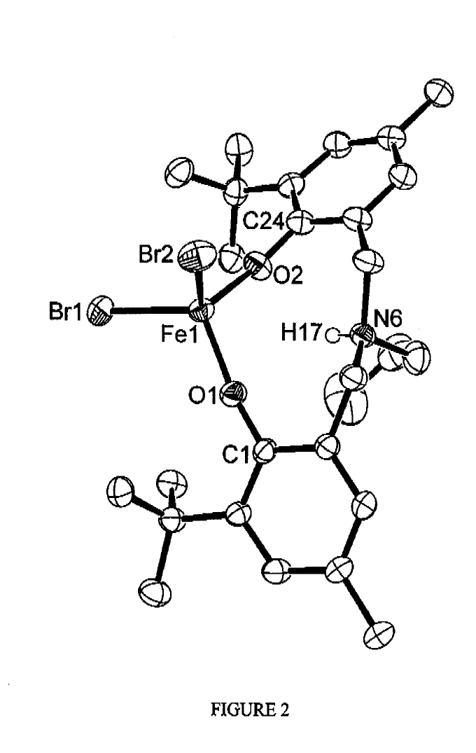

The temperature dependant magnetic behavior of {FeCl[O₂N]^{tBuMePr}}₂ was examined in the range of 2 to 300 K. Variable temperature magnetic studies show a very weak decrease in the magnetic moment vs. temperature from 5.50 μ_{B} at 300 K to 5.14 μ_{B} at 40 K (FIG. 3). Below this temperature, the moment drops rapidly to 3.15 μ_{B} at 2 K. The room temperature moment is slightly lower than expected for a high spin d⁵ ion. No maximum is observed in the plot of susceptibility, χ, vs. T suggesting the absence of significant antiferromagnetic exchange between iron centres. A plot of 1/χ vs. T gives a straight line indicating {FeCl[O₂N]^{tBuMePr}}₂ obeys the Curie-Weiss law.

Magnetic moments of complexes {FeCl[O₂N]^{tButBuPr}}₂, {FeCl[O₂N]^{tBuMeBenzyl}}₂ and FeBr[O₂]^{tBuMePr} were measured in solution by Evans' method at room temperature. All compounds exhibit moments of 5.8 μ_{B} per iron centre, consistent with high spin d⁵ ions. The iron centres in these complexes were shown to possess five strong metal-ligand interactions.

UV-Visible Spectroscopy

The synthesized iron complexes are intensely purple-coloured solids and exhibit strong, solvent dependant, bands in their UV-vis spectrum. The highest energy bands (<300 nm) result from ligand π→π* transitions. Indeed, the UV-vis spectrum of the unmetallated ligand in methanol exhibits strong bands in this region. Other strong bands occur in the UV region (≈275-350 nm), which are assigned to charge-transfer from the out-of-plane p_{π} orbital (HOMO) of the phenolate oxygen to the half-filled dₓ2-_{y}2/d_{z}2 orbital of high spin iron(III). The lowest energy bands (visible region) arise from charge-transfer transitions from the in-plane p_{π} orbital of the phenolate to the half-filled d_{π*} orbital of iron(III). These bands show the strongest solvent dependance and their λₘₐₓ values shift to longer wavelength according to the trend: pentane (470 nm)<MeCN≈THF (490 nm)<MeOH (610 nm).

### Example 2

### Preparation and Characterization of Tetradentate Ligands and Complexes

All manipulations and handling of ligands and iron complexes were performed in air. Reagents were purchased and used without further purification. The amine-bis(phenolate)-ether ligands, H₂[L₁] to H₂[L₅], (shown in Scheme 4 below) were prepared by modified literature procedures (e.g. as found in Kerton et al. 2008 Can. J. Chem. Vol. 86, p. 435) employing Mannich condensation of 2,4-dichlorophenol or 2,4-difluorophenol, formaldehyde and the corresponding primary amine in water, as described below.

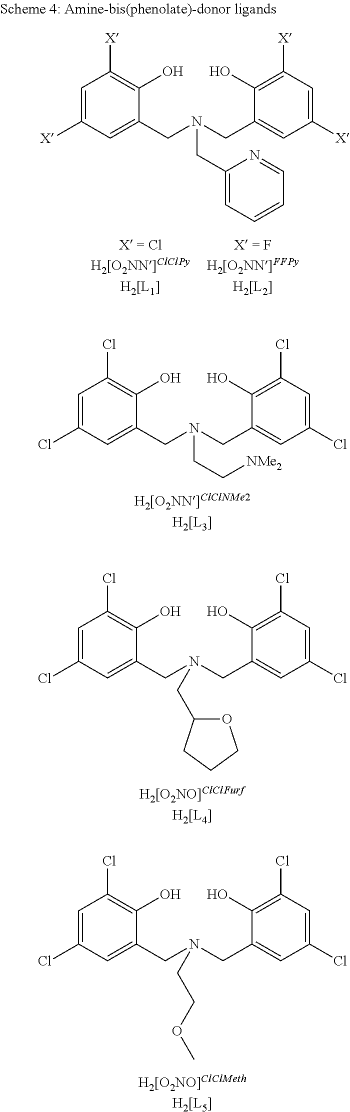

To a mixture of 2,4-dichlorophenol (20.06 g, 0.123 mol) and 37% aqueous formaldehyde (10.00 mL, 0.123 mol) in water (50 mL) was slowly added aminomethylpyridine (6.60 g, 0.061 mol), which resulted in a cloudy suspension. The mixture was stirred and heated to reflux for 12 b. Upon cooling, a large quantity of pale orange solid formed. The solvents were decanted and the remaining solid residue was washed with cold methanol to give an analytically pure white powder. Yield: 20.00 g (70%). Crystalline product was obtained by slow cooling of a hot diethyl ether solution. ¹H NMR (500 MHz, CDCl₃, 298 K): δ 8.69 (d, ³J_{H1-H2}=5.8 Hz, PyH, 1H); 7.78 (ddd, ³J_{H3-H4}=8.2 Hz, ³J_{H3-H2}=7.0 Hz, ⁴J_{H3-H1}=1.8 Hz, PyH, 1H); 7.34 (dd, ³J_{H2-H1}=5.8 Hz, ³J_{H2-H3}=7.0 Hz, PyH, 1H); 7.28 (s, ArH, 2H); 7.16 (d, ³J_{H4-H3}=8.2 Hz, PyH, 1H); 6.94 (s, ArH, 2H); 3.85 (s, CH₂, 2H); 3.79 (s, CH, 4H). ¹³C{¹H}NMR (125 MHz, CDCl₃, 298 K): δ 155.1 (Py), 155.5 (ArC—OH), 148.6 (Py), 138.7 (Py), 130.1 (Ar), 128.9 (Ar), 124.3 (Ar), 124.1 (Ar), 123.6 (Py), 123.1 (Py), 122.3 (Ar), 56.5 (CH₂), 55.9 (CH₂). IR (cm⁻¹): 3350 (OH); 2955 (C—H); 1603 (C═C, phenyl ring). Anal. Calcd for C₂₀H₁₆Cl₄N₂O₂: C, 52.43; H, 3.52; N, 6.11. Found C, 52.45; H, 3.50; N, 6.10.

Synthesis of Ligand H₂[O₂NN′]^{FFPy} (H₂[L₂])

To a solution of 2,4-difluorophenol (4.00 g, 0.031 mol) and 37% aqueous formaldehyde (2.50 mL, 0.031 mol) in water (50 mL) was added aminomethylpyridine (1.663 g, 0.015 mol), which formed a cloudy precipitate and a yellow oil. The mixture was stirred and heated to reflux for 12 h. Upon cooling, a large quantity of yellow oil formed. The solvents were decanted and the remaining oily residue was triturated with cold methanol under ultrasound to give an analytically pure white powder. Yield: 3.52 g (58%). ¹H NMR (500 MHz, (CD₃)₂CO, 298 K): δ 10.72 (s, OH, 1H); 8.68 (d, ³J_{H1-H2}=5.8 Hz, PyH, 1H); 7.91 (ddd, ³J_{H3-H4}, 8.2 Hz, ³J_{H3-H2}=7.0 Hz, ⁴J_{H3-H1}=1.8 Hz, PyH, 1H); 7.46 (dd, ³J_{H2-H1}=5.8 Hz, ³J_{H2-H3}=7.0 Hz, PyH, 1H); 7.43 (s, ArH, 2H); 6.93 (d, ³J_{H4-H3}=8.2 Hz, PyH, 1H); 6.90 (s, ArH, 2H); 4.01 (s, CH₂, 2H); 3.91 (s, CH₂, 4H). ¹³C{¹H}NMR (125 MHz, (CD₃)CO, 298 K): δ 155.1 (Py), 158.5 (ArC—OH), 149.2 (Py), 139.7 (Py), 130.9 (Ar), 123.2 (Ar), 124.9 (Ar), 124.6 (Ar), 124.1 (Py), 123.8 (Py), 122.9 (Ar), 57.0 (CH₂), 56.5 (CH₁). IR (cm⁻¹): 3350 (OH); 2955 (C—H); 1600 (C═C, phenyl ring). Anal. Calcd for C₂₀H₁₆F₄N₂O₂: C, 61.22; H, 4.11; N, 7.14. Found C, 61.30; H, 4.15; N, 7.10.

Synthesis of Ligand H₂[O₂NN′]^{ClClNMe2} (H₂[L₃])

To a solution of 2,4-dichlorophenol (20.07 g, 0.123 mol) and 37% aqueous formaldehyde (10.00 mL, 0.123 mol) in water (50 mL) was added N,N-dimethylethylenediamine (6.70 mL, 0.062 mol), which formed a cloudy precipitate and a yellow oil. The mixture was stirred and heated to reflux for 12 h. Upon cooling, a large quantity of yellow oil formed. The solvents were decanted and the remaining oily residue was triturated with cold methanol under ultrasound to give an analytically pure white powder. Yield: 17.54 g (65%). Spectroscopic analysis is identical to that previously reported.

Synthesis of Ligand H₂[O₂NO]^{ClClFwf} (H₂[L₄])

To a mixture of 2,4-dichlorophenol (20.00 g, 0.123 mol) and 37% aqueous formaldehyde (10,00 mL, 0.123 mol) in water (50 mL) was slowly added tetrahydrofurfurylamine (6.18 g, 0.061 mol), which resulted in a white precipitate. The mixture was stirred and heated to reflux for 12 h. Upon cooling, a large quantity of white solid and yellow oil formed. The solvents were decanted, and the remaining residues were triturated with cold methanol under ultrasound to give an analytically pure white powder. Yield: 19.30 g (70%). ¹H NMR (500 MHz, CDCl₃, 298 K, Labelled resonances correspond to diagram below): δ 8.29 (s, OH^{a}, 2H); 7.26 (s, ArH^{b}, 2H,); 6.94 (s, ArH^{c}, 2H,); 4.19 (m, CH^{f}, 1H,); 3.98 (m, CH₂^{h}, 2H,); 3.73 (s, CH₂^{d}, 4H); 2.60 (m, CH₂^{e}, 2H,); 1.92 (m, CH₂^{g}, 4H). ¹³C{¹H}NMR (125 MHz, CDCl₃, 298 K): δ 151.6 (ArCOH); 129.6 (ArCCl); 129.0 (ArCCl); 124.8 (ArCH); 124.5 (ArCH); 122.4 (ArC); 78.0 (CH); 69.0 (CH₂); 57.1 (CH₂); 56.6 (CH₂); 30.3 (CH₂); 25.6 (CH₂). IR (cm⁻¹): 3350 (OH); 2955 (C—H); 1603 (C═C, phenyl ring). Anal. Calcd for C₁₉H₁₉Cl₄NO₃: C, 50.58; H, 4.24; N, 3.10. Found C, 50.45; H, 4.30; N, 3.10.

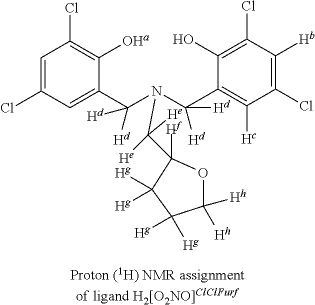

Synthesis of Ligand H₂[O₂NO]^{ClClMeth} (H₂[L₅])

To a solution of 2,4-dichlorophenol (20.07 g, 0.123 mol) and 37% aqueous formaldehyde (10.00 mL, 0.123 mol) in water (50 mL) was added (2-methoxy)ethylamine (5.40 mL, 0.062 mol), which formed a cloudy precipitate. The mixture was stirred and heated to reflux for 12 h. Upon cooling, a large quantity of yellow oil formed. The solvents were decanted and the remaining oily residue was triturated with cold methanol under ultrasound to give an analytically pure white powder. Yield: 16.54 g (62%). Spectroscopic analysis is identical to that previously reported.

Preparation of Iron Complexes

Anhydrous FeCl₃ (97%) and FeBr₃ (99%) were purchased and used without further purification. The desired iron(III) complexes were obtained by dropwise addition of a THF solution of FeX₃ (X=Cl, Br) to a THF solution of the ligand at room temperature to yield a dark blue mixture. NEt₃ in methanol is added to neutralize the resulting solution. The complexes FeX[L₁](1, X=Cl; 2, X=Br), FeX[L₂](3, X=Cl; 4, X=Br), FeX[L₃](5, X=Cl; 6, X=Br), FeX[L₄](7, X=Cl; 8, X=Br) and FeX[L₅](9, X=Cl; 10, X=Br) are obtained as paramagnetic dark indigo powders that give analytically pure products upon recrystallization from methanol or acetone.

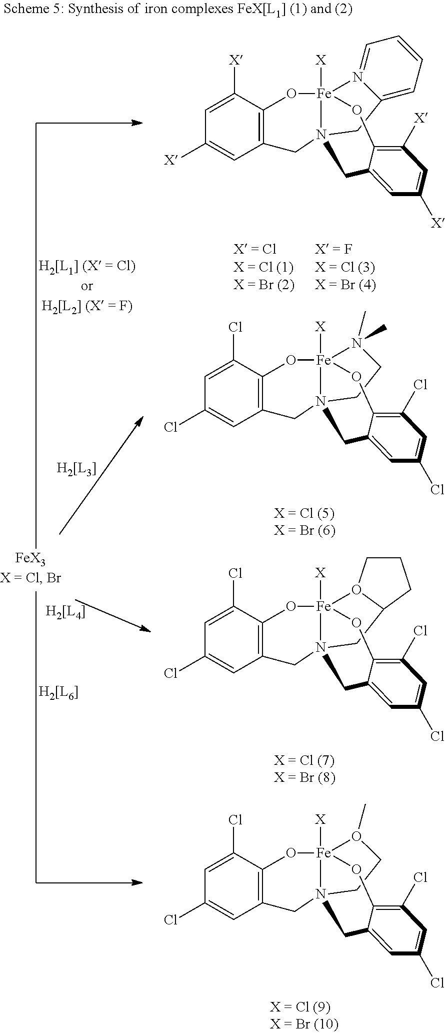

To a THF solution of recrystallized H₂[O₂NN′]^{ClClPy}, H₂[L₁], (3.00 g, 6.55 mmol) was added a solution of anhydrous FeX₃ (6.55 mmol) in THF resulting in an intense violet/blue solution. To this solution was added triethylamine (1.32 g, 13.0 mmol) and the resulting mixture was stirred for 2 h. Solvent was removed under vacuum and the residue was extracted with toluene and filtered through Celite three times. Removal of solvent under vacuum yielded analytically pure dark-blue products. Yield 1: 3.19 g (89%); yield 2: 3.30 g (85%). Crystals suitable for X-ray diffraction were obtained by slow evaporation of a solution of 2 in a 1:1 mixture of hexanes and chloroform to give 2.H₂O. Characterization for 1: Anal. Calcd for C₂₀H₁₄Cl₅FeN₂O₂: C, 43.88; H, 2.58; N, 5.12. Found C, 43.95; H, 2.65; N, 4.93. MS (MALDI-TOF) m/z (%, ion): 544.89 (20, [M]⁺), 509.92 (100, [M-Cl]⁺). UV-vis λₘₐₓ, nm (ε): (methanol) 480 (8,000), 325 (14,500), 280 (16,000). μ_{eff} (solution, 25° C.) 5.9 μ_{B}. Characterization for 2: Anal. Calcd for C₂₀H₁₄BrCl₄FeN₂O₂: C, 40.58; H, 2.38; N, 4.73. Found C, 40.65; H, 2.35; N, 4.80. MS (MALDI-TOF) m/z (%, ion): 588.83 (20, [M]⁺), 509.92 (100, [M-Br]⁺). UV-vis λₘₐₓ, nm (ε): (methanol) 480 (8,000), 325 (14,500), 280 (16,000). μ_{eff} (solution, 25° C.) 5.8 μ_{B}.

Synthesis of Iron Complexes FeX[L₂](3) and (4)

To a THF solution of recrystallized H₂[O₂NN′]^{FFPy}, H₂[L₂], (3.00 g, 6.52 mmol) was added a solution of anhydrous FeX₃ (6.52 mmol) in THF resulting in an intense violet/blue solution. To this solution was added triethylamine (1.32 g, 13.0 mmol) and the resulting mixture was stirred for 2 h. Solvent was removed under vacuum and the residue was extracted with toluene and filtered through Celite three times. Removal of solvent under vacuum yielded analytically pure dark-blue products. Yield 3: 2.20 g (70%); yield 4: 2.95 g (86%). Characterization for 3: Anal. Calcd for C₂₀H₁₄ClF₄FeN₂O₂: C, 49.88; H, 2.93; N, 5.82. Found C, 49.95; H, 2.95; N, 5.93. MS (MALDI-TOF) m/z (%, ion): 481.00 (20, [M]⁺), 446.03 (100, [M-Cl]⁺). UV-vis λₘₐₓ, nm (ε): (methanol) 480 (8,000), 325 (14,500), 280 (16,000). μ_{eff} (solution, 25° C.) 5.9 μ_{B}. Characterization for 4: Anal. Calcd for C₂₀H₁₄BrF₄FeN₂O₂: C, 45.66; H, 2.68; N, 5.32. Found C, 45.65; H, 2.65; N, 5.32. MS (MALDI-TOF) m/z (%, ion): 524.95 (20, [M]⁺), 446.03 (100, [M-Br]⁺). UV-vis λₘₐₓ, nm (ε): (methanol) 480 (8,000), 325 (14,500), 280 (16,000). μ_{eff} (solution, 25° C.) 5.8 μ_{B}.

Synthesis of Iron Complexes FeX[L₃](5) and (6)

To a THF solution of recrystallized H₂[O₂NN′]^{ClClNMe2}, H₂[L₃], (2.86 g, 6.55 mmol) was added a solution of anhydrous FeX₃ (6.55 mmol) in THF resulting in an intense violet/blue solution. To this solution was added triethylamine (1.32 g, 13.0 mmol) and the resulting mixture was stirred for 2 b. Solvent was removed under vacuum and the residue was extracted with toluene and filtered through Celite three times. Removal of solvent under vacuum yielded analytically pure dark-blue products. Yield 5: 3.11 g (90%); yield 6: 3.00 g (80%). Characterization for 5: Anal. Calcd for C₁₈H₁₈Cl₅FeN₂O₂: C, 40.99; H, 3.44; N, 5.31. Found C, 40.95; H, 3.55; N, 5.33. MS (MALDI-TOF) m/z (%, ion): 524.92 (20, [M]⁺), 489.95 (100, [M-Cl]⁺). UV-vis λₘₐₓ, nm (ε): (methanol) 480 (8,000), 325 (14,500), 280 (16,000). μ_{eff} (solution, 25° C.) 5.9 μ_{B}. Characterization for 6: Anal. Calcd for C₁₈H₁₈BrCl₄FeN₂O₂: C, 37.80; H, 3.17; N, 4.90. Found C, 37.85; H, 3.15; N, 4.80. MS (MALDI-TOF) m/z (%, ion): 568.87 (20, [M]⁺), 489.95 (100, [M-Br]⁺). UV-vis λₘₐₓ, nm (ε): (methanol) 480 (8,000), 325 (14,500), 280 (16,000). μ_{eff} (solution, 25° C.) 5.8 μ_{B}.

Synthesis of Iron Complexes FeX[L₄] (7) and (8)

To a THF solution of recrystallized H₂[O₂NO]^{ClClFurf}, H₂[L₄], (2.96 g, 6.55 mmol) was added a solution of anhydrous FeX₃ (6.55 mmol) in THF resulting in an intense violet/blue solution. To this solution was added triethylamine (1.32 g, 13.0 mmol) and the resulting mixture was stirred for 2 h. Solvent was removed under vacuum and the residue was extracted with toluene and filtered through Celite three times. Removal of solvent under vacuum yielded analytically pure dark-blue products. Yield 5: 2.48 g (70%); yield 6: 2.68 g (70%). Characterization for 5: Anal. Calcd for C₁₉H₁₇Cl₅FeNO₃: C, 42.22; H, 3.17; N, 2.59. Found C, 42.35; H, 3.15; N, 2.53. MS (MALDI-TOF) m/z (%, ion): 537.90 (20, [M]⁺), 502.93 (100, [M-Cl]⁺). UV-vis λₘₐₓ, nm (ε): (methanol) 480 (8,000), 325 (14,500), 280 (16,000). μ_{eff} (solution, 25° C.) 5.9 μ_{B}. Characterization for 6: Anal. Calcd for C₁₉H₁₇BrCl₄FeNO₃: C, 39.02; H, 2.93; N, 2.39. Found C, 38.85; H, 3.10; N, 2.40. MS (MALDI-TOF) m/z (%, ion): 581.85 (20, [M]⁺), 489.95 (100, [M-Br]⁺). UV-vis λₘₐₓ, nm (ε): (methanol) 480 (8,000), 325 (14,500), 280 (16,000). μ_{eff} (solution, 25° C.) 5.8 μ_{B}.

Synthesis of Iron Complexes FeX[L₅](9) and (10)

To a THF solution of recrystallized H₂[O₂NO]^{ClClMeth}, H₂[L₄], (2.78 g, 6.55 mmol) was added a solution of anhydrous FeX₃ (6.55 mmol) in THF resulting in an intense violet/blue solution. To this solution was added triethylamine (1.32 g, 13.0 mmol) and the resulting mixture was stirred for 2 h. Solvent was removed under vacuum and the residue was extracted with toluene and filtered through Celite three times. Removal of solvent under vacuum yielded analytically pure dark-blue products. Yield 9: 3.03 g (90%); yield 10: 3.46 g (95%). Characterization for 9: Anal. Calcd for C₁₇H₁₅Cl₅FeNO₃: C, 39.69; H, 2.94; N, 2.72. Found C, 39.85; H, 3.11; N, 2.53. MS (MALDI-TOF) m/z (%, ion): 511.88 (20, [M]⁺), 476.92 (100, [M-Cl]⁺). UV-vis λₘₐₓ, nm (ε): (methanol) 480 (8,000), 325 (14,500), 280 (16,000). μ_{eff} (solution, 25° C.) 5.9 μ_{B}. Characterization for 10: Anal. Calcd for C₁₇H₁₅BrCl₄FeNO₃: C, 36.53; H, 2.71; N, 2.51. Found C, 36.55; H, 2.83; N, 2.50. MS (MALDI-TOF) m/z (%, ion): 555.83 (20, [M]⁺), 489.95 (100, [M-Br]⁺). UV-vis λₘₐₓ, nm (ε): (methanol) 480 (8,000), 325 (14,500), 280 (16,000). μ_{eff} (solution, 25° C.) 5.8 μ_{B}.

Characterization of Iron Complexes

The ¹H NMR spectra of these iron compounds show shifted and broadened resonances as a result of their paramagnetic nature. All the iron(III) tetradentate amine-bis(phenolate) complexes have magnetic moments in solution in the range of 5.8-5.9 μ_{B} obtained by Evans' method at room temperature, consistent with high-spin d⁵ ions. MALDI-TOF mass spectrometry was useful in characterizing these paramagnetic complexes. When prepared using an anthracene matrix, masses were observed corresponding to the parent and characteristic fragment ions, but in all complexes the parent ion corresponding to FeX[Lₙ] is relatively weak. The halide ion is only weakly coordinated to the metal and therefore the reference peak corresponds to the loss of halide, [M-X]⁺, namely Fe[L₁]⁺. The identity of the fragments was further confirmed by matching the isotopic patterns of the relevant peaks.

MALDI-TOF MS spectra were recorded on an Applied Biosystems Voyager DE-PRO equipped with a reflectron, delayed ion extraction and high performance nitrogen laser (337 nm). Samples were prepared at a concentration of 0.03 mg L⁻¹ in methanol. Anthracene was used as the matrix, which was mixed at a concentration of 0.03 mg L⁻¹. UV-vis spectra were recorded on an Ocean Optics USB4000+ fiber optic spectrophotometer. Room temperature magnetic moments were determined in solution by Evans' NMR method. Elemental analyses were carried out by Chemisar Laboratories Inc., Guelph, ON, Canada or Canadian Microanalytical Service Ltd, Delta, BC, Canada.

Combustion analysis of the amorphous powder obtained prior to recrystallization confirms the formation of complexes that can be formulated as FeX[Lₙ]. Microcrystalline samples of these materials were used for further characterization and catalysis studies as discussed below.

X-Ray Crystallography of Iron Complexes

Single crystals of the iron complexes were isolated as described below. The data was processed and corrected for Lorentz and polarization effects and absorption. Neutral atom scattering factors for all non-hydrogen atoms were taken from the International Tables for X-ray Crystallography (D. T. Cromer, J. T. Waber, International Tables for X-ray Crystallography, The Kynoch Press, Birmingham, UK, 1974, Vol. IV.). The structure was solved by direct methods using SIR92 (Altomare et al. 1994 J. Appl. Cryst. Vol. 27, p. 435) and expanded using Fourier techniques (DIRDIF99) (Beurskens et al., DIRDIF99, University of Nijmegen, Netherlands, 1999).

All non-hydrogen atoms were refined anisotropically. Hydrogen atoms were included in calculated positions, with the exception of two hydroxyl hydrogens that were found in the difference map, with isotropic parameters set twenty percent greater than those of their bonding partners. Anomalous dispersion effects were included in Fcalc (Ibers J A et al. 1964 Acta Crystallogr. Vol. 17, p. 781); the values for Δf′ and Δf″ were those of Creagh and McAuley (Tables for Crystallography, Kluwer Academic Publishers, Boston, 1992, Vol. Vol. C, Table 4.2.6.8, pp. 219-222.). The values for the mass attenuation coefficients are those of Creagh and Hubbell (International Tables for Crystallography, Kluwer Academic Publishers, Boston, 1992, Vol. Vol. C, Table 4.2.4.3, pp. 200-206). All calculations were performed using the CrystalStructure crystallographic software package except for refinement, which was performed using SHELXL-97. The data were reduced and corrected for absorption. The structure was refined by full-matrix least squares on F²(SHELXTL). All non-hydrogen atoms were refined using anisotropic displacement parameters. Hydrogen atoms were included in calculated positions and refined using a riding model except those for the water molecule which were found in Fourier difference maps and refined using isotropic thermal parameters. Structural illustrations were created using ORTEP-III for Windows.

Characterization of Ligand H₂[L₁]

Single crystals of H₂[L₁] suitable for X-ray diffraction were obtained from a saturated chloroform or acetone solution, Suitable crystals were selected and mounted on glass fibers using Paratone-N oil and freezing to −135±1° C. A hemisphere of data was collected on a Bruker AXS P4/SMART 1000 diffractometer using ω and θ scans with a scan width of 0.3° and 30 s exposure times. The detector distance was 5 cm.

Crystallographic data for compound H₂[L₁] is summarized in Table 2. Data collections for H₂[L₁] were performed on a Rigaku AFC8-Satum 70 equipped with a CCD area detector and an X-Stream 2000 low temperature system, using graphite monochromated Mo-Kα radiation (α=0.71073 Å). The structure of H₂[L₁] (obtained at 138 K) is shown in FIG. 3.

TABLE 2
Selected Bond Lengths (Å) and Angles (°) for H₂[L₁]

| | | | |
|---|---|---|---|
| Cl(1)-C(4) | 1.742(2) | O(2)-C(20)-C(19) | 119.40(17) |
| Cl(2)-C(6) | 1.740(2) | O(2)-C(20)-C(15) | 121.77(17) |
| Cl(3)-C(19) | 1.734(2) | N(2)-C(13)-C(12) | 123.15(19) |
| Cl(4)-C(17) | 1.748(2) | N(1)-C(14)-C(15) | 110.71(15) |
| O(1)-C(3) | 1.346(2) | N(1)-C(1)-C(2) | 111.99(15) |
| O(2)-C(20) | 1.355(2) | N(1)-C(8)-C(9) | 113.89(15) |
| N(1)-C(8) | 1.469(2) | N(2)-C(9)-C(10) | 121.58(18) |
| N(1)-C(1) | 1.480(2) | N(2)-C(9)-C(8) | 119.25(16) |
| N(1)-C(14) | 1.482(2) | C(8)-N(1)-C(1) | 111.24(15) |
| N(2)-C(9) | 1.339(2) | C(8)-N(1)-C(14) | 110.36(14) |
| N(2)-C(13) | 1.345(3) | C(1)-N(1)-C(14) | 109.89(14) |
| | | C(9)-N(2)-C(13) | 118.38(17) |

Ligand precursor, H₂[L₁], displays intramolecular hydrogen-bonding between the phenolic hydroxyl group O(1)-H and the pyridyl nitrogen N(2), and between the second phenolic hydroxyl group, O(2)-H, and the amine nitrogen N(1) [O(1) . . . N(2) 2.699 Å and O(2) . . . N(1) 2.720 Å], which gives the molecule an orientation similar to that observed in metal complexes of this class of ligand. No intermolecular hydrogen bonding is observed. ¹H and ¹³C{¹H}NMR analyses of H₂[L₁] are consistent with the solid-state structure but show that in solution the hydrogen bonding interactions are easily broken allowing free rotation of the phenol fragments. The methylene protons located between the amine nitrogen and the phenol groups appear as singlets in CDCl₃ or de-acetone, whereas restricted rotation of the methylene group would lead to diastereotopic protons. The bond lengths and angles around each atom are consistent with those found in the structures of related ligands. (R. R. Chowdhury et al. Chem. Commun. (2008) 94.; E. Safaei et al. Eur. J. Inorg. Chem. (2007) 2334.; C. Lorber et al. Eur. J. Inorg. Chem. (2005) 2850.; and D. Maity et al. Inorg. Chim. Acta 359 (2006) 3197)

Iron Complex 2.H₂O {FeBr[O₂NN′]^{ClClPy}(H₂O)}

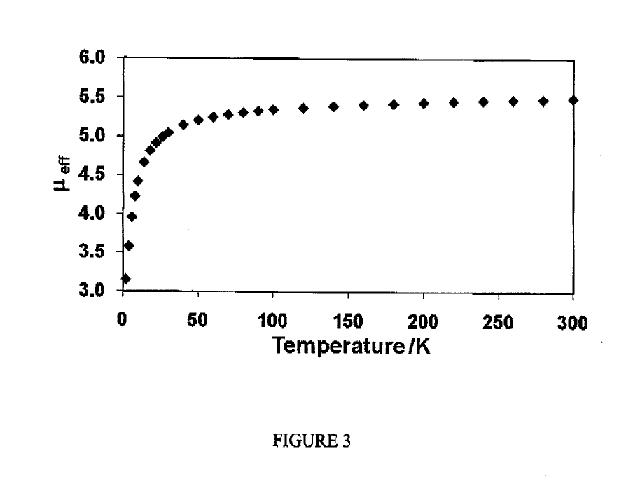

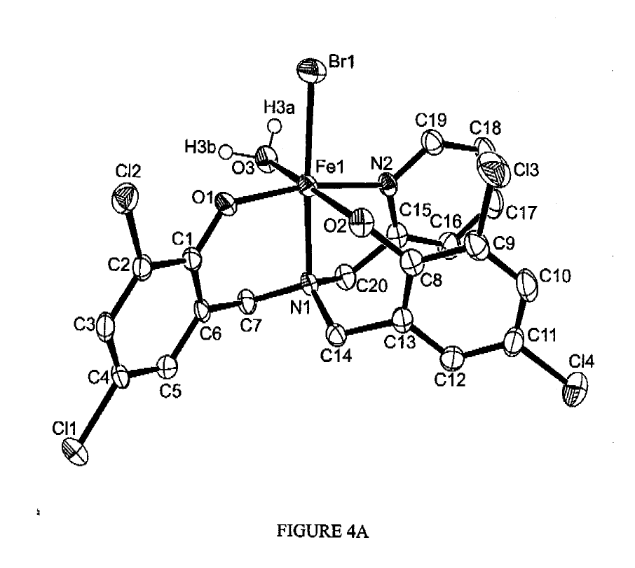

Single crystals suitable for X-ray diffraction were obtained by slow evaporation of a solution of FeBr[O₂NN′]^{ClClPy}(H₂O) in a mixture of hexanes and chloroform in air at room temperature. The structure is shown in FIG. 4a and selected bond lengths and angles are given in Table 2. The coordination geometry around the iron atom is distorted octahedral as a result of the addition of a water ligand. This water ligand results from adventitious water present in the solvent of recrystallization or atmospheric moisture.

The molecular structure (ORTEP) of FeBr[O₂NN′]^{ClClPy}(H₂O) is shown in aa. FeBr[O₂NN′]^{ClClPy}(H₂O) exhibits a six-coordinate geometry constituted by two cis-coordinated phenolate oxygen atoms (O(1), O(2)) and the cis-coordinated amine and pyridine nitrogen donors (N(1) and N(2)). The bromide ion and aquo ligand reside in cis positions, with the bromide residing trans to the central amine nitrogen and the aquo oxygen occupying a position trans to one of the phenolate oxygen donors. The molecule therefore possesses C symmetry. The cis-disposed Fe—O(phenolate) bonds are significantly different (Fe—O(1), 1.879 (3); Fe—O(2), 1.970(3) Å), with the longer bond being found for the phenolate oxygen located trans to the water ligand. The Fe—Br bond distance is 2.4241(7) Å, and Fe—N distances are 2.261(3) and 2.179(3) Å for Fe(1)-N(1) and Fe(1)-N(1), respectively. The Fe—O bond distance of the water ligand is 2.148(3) Å and is consistent with bond lengths observed in related octahedral iron(III) complexes bearing non-bridging aquo ligands. The Fe—O—C bond angles are asymmetric, namely C(1)-O(1)-Fe(1) is 134.1(3)° and C(8)-O(2)-Fe(1) is 122.5(2)°. The smaller bond angle at O(2) (and possibly the slightly longer Fe(1)-O(2) bond) may be a result of intramolecular π-π stacking between the pyridine ring the phenolate ring. The distance between centroids is 3.519 Å with an angle of 26.04° between the planes of the two aromatic rings.

Furthermore, the structure exhibits intermolecular π-π stacking as well as intermolecular hydrogen bonding. The interplanar distance between stacked phenolate rings in a pair of complexes is approximately 3.55 Å (found between the centroid of ring A and the ipso carbon of ring B) and the two rings display an offset orientation. In addition to this intermolecular phenyl stacking, there exists a hydrogen bonding interaction between the water ligand of one molecule (the H-bond donor) and the phenolate oxygen (the H-bond acceptor) of a “partner” molecule giving an O . . . O interatomic distance of 2.688 Å between oxygen atoms. This is shown in FIG. 4b. This interaction results in the formation of hydrogen bonded linear “chains” in the solid state. Despite the absence of bulky groups in the 2-position in this reported complex (the phenolate groups possess para-nitro substituents), the pendant donor is a dimethylaminoethyl group, which occupies a sterically larger volume of space around the metal ion than the planar pyridine ring found in FeBr[O₂NN′]^{ClClPy}(H₂O).

TABLE 3
Selected Bond Lengths (Å) and Angles (°) for FeBr[O₂NN']^{ClClPy}(H₂O)

| | | | |
|---|---|---|---|
| Fe(1)-O(1) | 1.879(3) | O(1)-Fe(1)-O(2) | 99.52(13) |
| Fe(1)-O(2) | 1.970(3) | O(1)-Fe(1)-O(3) | 89.27(13) |
| Fe(1)-O(3) | 2.148(3) | O(2)-Fe(1)-O(3) | 167.49(13) |
| Fe(1)-N(2) | 2.179(3) | O(1)-Fe(1)-N(2) | 163.16(13) |
| Fe(1)-N(1) | 2.261(3) | O(2)-Fe(1)-N(2) | 87.68(12) |
| Fe(1)-Br(1) | 2.4241(7) | O(3)-Fe(1)-N(2) | 81.56(13) |
| Cl(1)-C(4) | 1.744(4) | O(1)-Fe(1)-N(1) | 88.31(12) |
| Cl(2)-C(2) | 1.745(4) | O(2)-Fe(1)-N(1) | 86.08(12) |
| Cl(3)-C(9) | 1.731(5) | O(3)-Fe(1)-N(1) | 85.30(13) |
| Cl(4)-C(11) | 1.731(5) | N(2)-Fe(1)-N(1) | 76.94(12) |
| N(1)-C(20) | 1.482(5) | O(1)-Fe(1)-Br(1) | 97.43(9) |
| N(1)-C(14) | 1.489(5) | O(2)-Fe(1)-Br(1) | 96.96(9) |
| N(1)-C(7) | 1.493(5) | O(3)-Fe(1)-Br(1) | 90.64(10) |
| N(2)-C(15) | 1.341(5) | N(2)-Fe(1)-Br(1) | 96.77(9) |
| N(2)-C(19) | 1.348(5) | N(1)-Fe(1)-Br(1) | 172.95(9) |
| O(1)-C(1) | 1.321(5) | C(1)-O(1)-Fe(1) | 134.1(3) |
| O(2)-C(8) | 1.339(5) | C(8)-O(2)-Fe(1) | 122.5(2) |

Electronic Spectroscopy of tetradentate amine-bis(phenolate) Iron Complexes

Electronic absorption spectra of all complexes in methanol show multiple intense bands in the UV and visible regions. In tetradentate amine-bis(phenolate) iron complexes 1-10, the absorption maxima observed in the near-UV regions (below 300 nm) are caused by π→π* transitions involving the phenolate units—absorptions in this region are observed in the spectra of the unmetallated ligand precursors. Intense bands (which appear as shoulders on the π→π* bands) are observed in the region between 300 and 375 nm and are assigned to charge transfer transitions from the out-of-plane p_{π} orbital (HOMO) of the phenolate oxygen to the half-filled dₓ_{²}_{y}_{²}/d_{z}_{²} orbital of high-spin Fe(III). Intense, lower energy bands between 450 and 700 nm in the visible region are proposed to arise from charge-transfer transitions from the in-plane p, orbital of the phenolate to the half-filled d, orbital of Fe(III) and account for the intense indigo blue/purple colour of the complexes. The halide ligands are anticipated to be labile in solution, hence a coordinating solvent such as methanol would clearly interact with the iron(III) centres influencing the ligand fields and, therefore, the electronic spectra.

### Example 3

### Cross-Coupling Catalysis with Tridentate amine-bis(phenolate) Iron Complexes

The catalytic ability of tridentate iron amine (bisphenolate) complexes was demonstrated using the complex {FeCl[O₂N]^{tBuMePr}}₂. The air-stable, single component iron(III) complex {FeCl[O₂N]^{tBuMePr}}₂ catalyzes the C(sp³)-C(sp²) bond forming reaction between aryl Grignard reagents and alkyl halides, including primary as well as cyclic or acyclic secondary alkyl chlorides. The synthesis and characterization of the tridentate amine (bisphenolate) iron complex employed in the cross coupling reactions as follows are described in Example 1.

The cross coupling reaction was performed according to the general scheme:

R₄—MgBr+R₅—Br→R₄—R₅

Unless otherwise stated, all manipulations were performed under an atmosphere of dry oxygen-free nitrogen by means of Schlenk techniques or using an MBraun LabmasterDP glove box. Anhydrous diethyl ether was purified using an MBraun Solvent Purification System. THF was stored over sieves and distilled from sodium benzophenone ketyl under nitrogen. Reagents were purchased and used without further purification. Grignard reagents were titrated prior to use and were also analyzed by GC-MS after being quenched with dilute HCl(to quantify biaryl complexes or other impurities present prior to their use in catalyst runs.

NMR spectra were recorded in CDCl₃ on Bruker Avance-500 or AvanceIII-300 spectrometers. MALDI-TOF MS spectra were recorded on an Applied Biosystems Voyager DE-PRO equipped with a reflectron, delayed ion extraction and high performance nitrogen laser (337 nm). Samples were prepared at a concentration of 0.03 mg L⁻¹ in methanol. Anthracene was used as the matrix, which was mixed at a concentration of 0.03 mg L⁻¹. UV-vis spectra were recorded on an Ocean Optics USB4000+ spectrophotometer. Elemental analyses were carried out by Canadian Microanalytical Service Ltd, Delta, BC, Canada. Magnetic susceptibility data were acquired in the solid state using a Quantum Designs MPMS5 SQUID magnetometer and in solution using Evans' NMR method. (E. M. Schubert, J. Chem. Ed., 1992, 69, 62) Crystal structures were solved on a AFC8-Saturn 70 single crystal X-ray diffractometer from Rigaku/MSC, equipped with an X-stream 2000 low temperature system. Gas chromatography mass spectrometry (GC-MS) analyses were performed using an Agilent Technologies 7890 GC system coupled to an Agilent Technologies 5975C mass selective detector (MSD). The chromatograph is equipped with electronic pressure control, split/splitless and on-column injectors, and an HP5-MS column.

All catalytic reactions were performed on a Radleys Carousel Reactor™. Twelve 45 mL reaction tubes were fitted with threaded Teflon caps equipped with valves for connection to the inert gas or vacuum supply of a Schlenk apparatus, and septa for the introduction of reagents. Microwave-heated reactions were performed using a Biotage Initiator™ Eight microwave synthesizer.

Cross-Coupling Catalysis: Method A

Procedure for cross-coupling at room temperature: Catalyst {FeCl[O₂N]^{tBuMePr}}₂ (50 mg, 0.05 mmol of {FeCl[O₂N]^{tBuMePr}}₂ or 0.1 mmol formula units of {FeCl[O₂N]^{tBuMePr}}₂ in CH₂Cl₂ (3 mL) was added to a 45 mL Radleys Carousel Reactor tube and the solvent removed in vacuo. To the catalyst were added Et₂O (5 mL), alkyl halide (2.0 mmol) and an ether solution of aryl Grignard reagent (4.0 mmol) was added dropwise under vigorous stirring (except for entries 11 to 15 where 8.00 mmol of Grignard was used). The resulting mixture was stirred for 30 minutes, then dodecane (2.0 mmol as internal standard) was added and the reaction quenched with 5 ml 1.0 M HCl(aq). The organic phase was extracted with Et₂O (5 mL) and dried over MgSO₄. The mixture was analyzed by GC-MS and NMR. NMR samples were prepared by careful removal of solvent under vacuum and dissolving the residue in CDCl₃.

Cross-Coupling Catalysis: Method B

Procedure for cross-coupling under microwave-heating: In a glove box, complex {FeCl[O₂N]^{tBuMePr}}₂ (50 mg, 0.1 mmol) and a magnetic stir bar were added to a Biotage™ microwave vial, which was sealed with a septum cap. A solution of alkyl halide (2.00 mmol) in Et₂O was injected into the vial, followed by 4.00 mmol of Grignard reagent in Et₂O (except for entry 14 where 8.00 mmol of Grignard was used). The mixture was heated in a Biotage Initiator™ Eight Microwave Synthesizer using the following parameters: time=10 min; temperature=100° C.; prestirring=off; absorption level=high; fixed hold time=on. Upon completion, 2.00 mmol of dodecane (internal standard) was added to the mixture followed by 5.0 mL of 1.0 M HCl(aq) to quench. The product yields were quantified by GC-MS (relative to standard curves) and in several cases by ¹H NMR.

Reactions performed at room temperature gave superior results to those conducted at lower temperatures. A small number of experiments also explored the use of microwave heating of diethyl ether solutions to 100° C. to improve reaction yields.

The results of this study are summarized in Table 4 below.

TABLE 4
Cross-coupling of aryl Grignard with alkyl halides catalyzed by {FeCl[O₂N]^{tBuMePr}}₂^{a}

| Entry | ArMgBr | Alkyl halide | Product | Yield (%) [symbol] |
|---|---|---|---|---|
| 1 | Ph | 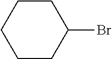 | 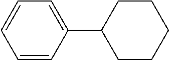 [SMILES: c1ccc(C2CCCCC2)cc1] | >95^{d} |
| 2 | m-Anisyl | 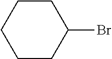 | 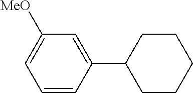 [SMILES: COc1cccc(C2CCCCC2)c1] | >95 |
| 3 | p-FPh | 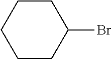 | 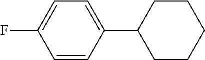 [SMILES: Fc1ccc(C2CCCCC2)cc1] | >95 |
| 4 | 2,6-Me₂Ph | 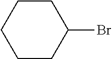 | 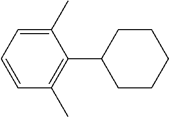 | Trace^{e} |
| 5 | 1-Naphthyl | 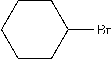 | 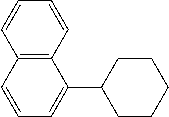 [SMILES: c1ccc2c(C3CCCCC3)cccc2c1] | 36 |
| 6 | p-Tolyl | 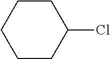 | 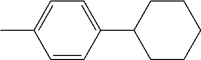 | 47 |
| 7 | o-Tolyl | 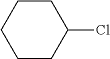 | 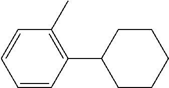 | 86 |
| 8 | p-Anisyl | 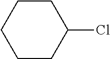 | 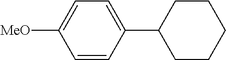 [SMILES: COc1ccc(C2CCCCC2)cc1] | 22 26^{f} 91 [symbol] 0^{g} |
| 9 | o-Tolyl | 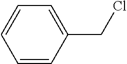 | 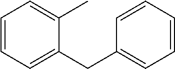 | >95 |
| 10 | 2,6-Me₂Ph | 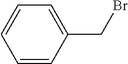 | 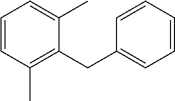 | 78 |
| 11 | o-Tolyl | 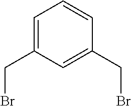 | 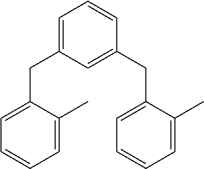 [SMILES: Cc1ccccc1Cc1cccc(Cc2ccccc2C)c1] | >95 |
| 12 | 2,6-Me₂Ph | 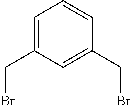 | 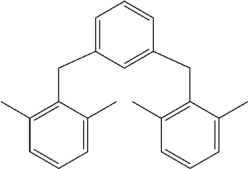 | 19 |
| 13 | o-Tolyl | 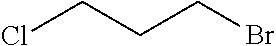 | 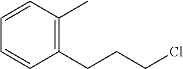 | 61 |
| 14 | p-FPh | 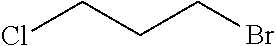 | 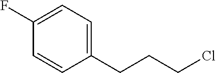 [SMILES: Fc1ccc(CCCCl)cc1] | 28^{e} |
| 15 | o-Tolyl | 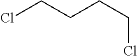 | 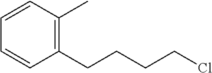 | 90 |
| 16 | o-Tolyl | n-C₈H₁₇Br | 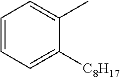 | 85^{d} |
| 17 | 2,6-Me₂Ph | n-C₈H₁₇Br | 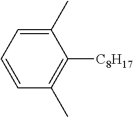 | Trace 94^{e} |
| 18 | o-Tolyl | 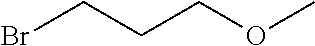 | 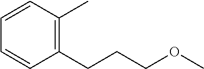 | 19 |
| 19 | o-Tolyl | 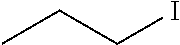 | 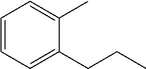 | 76 |
| 20 | p-FPh | 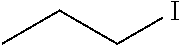 | 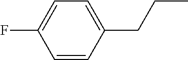 | 67 |
| 21 | 2,6-Me₂Ph | 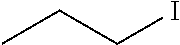 | 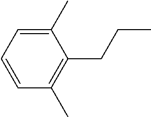 | Trace 88^{e} |
| 22 | 2,6-Me₂Ph | 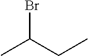 | 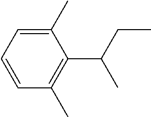 | Trace^{e} |
| 23 | p-Anisyl | 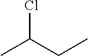 | 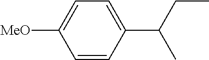 | 35 37^{e} |
| 24 | o-Tolyl | 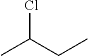 | 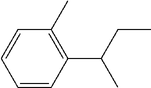 | 20 30^{e} 30^{h} |
| 25 | Ph | 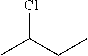 | 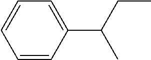 | 36 58^{e} |
| 26 | p-Tolyl | 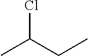 | 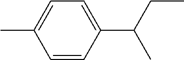 | 64 |
| 27 | o-Tolyl | 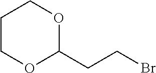 | 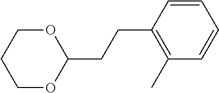 | 61 |
| 28 | o-Tolyl | 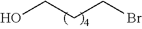 | 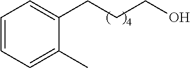 | 54 |
| 29 | o-Tolyl | 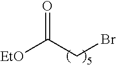 | 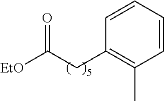 | 91 |
| 30 | p-FPh | 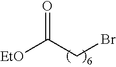 | 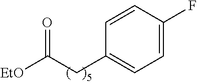 | 30 32 [symbol] |
| 31 | o-Tolyl | 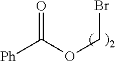 | 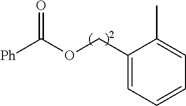 | 0 0 [symbol] |
| 32 | Ph | 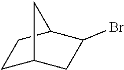 | 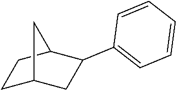 [SMILES: c1ccc(C2CC3CCC2C3)cc1] (exo:endo = 95:5) | 93 99^{e} |

^{a}See description herein for general procedure using 5.0 mol % (0.05 mmol) of iron complex and 2 equiv. Grignard reagent per halide functional group.
^{b}Yield determined by GC using dodecane as the internal standard.
^{c}Yield given for reactions performed in Et₂O for 30 min at 22° C. unless otherwise noted.
^{d}Reaction performed using 6.00 mmol of alkyl halide.
^{e}Microwave heating for 10 min at 100° C.
^{f}Performed for 30 min at 40° C.
^{g}Microwave heating for 10 min at 100° C. in the absence of iron complex.
^{h}Microwave heating for 10 min at 180° C. [symbol]
indicates data missing or illegible when filed

The system shows improved reactivity for sterically demanding nucleophiles, such as 2,6-dimethylphenylmagnesium bromide, and has demonstrated that diarylmethane motifs can be obtained using both benzyl bromides and chlorides.

### Example 4

### Cross-Coupling Catalysis with tetradentate amine (bisphenolate) Iron Complexes

Grignard cross-coupling reactions were carried out using tetradentate amine

(bisphenolate) iron complexes. The synthesis and characterization of the tetradentate amine-bis(phenolate) iron complexes employed in the following cross-coupling reactions are described in Example 2 above.

Catalyst (iron complex) (0.1 mmol, 5.0 mol %) in CH₂Cl₂ (3 mL) was added to a Schlenk flask followed by removal of the solvent in vacuo. To the catalyst were added Et₂O (5 mL), alkyl halide (2.0 mmol) and dodecane (2.0 mmol as internal standard) and the solution was stirred at room temperature. Grignard (4.0 mmol) was added and the resulting mixture was stirred for 30 minutes. The reaction was quenched with HCl (aq., 2 M, 5 mL) and the organic phase was extracted with Et₂O (1×5 mL) and dried over MgSO₄. The mixture was analyzed by GC-MS and quantified using ¹H NMR and/or GC. NMR samples were prepared by careful removal of solvent under vacuum and dissolving the residue in CDCl₃.

TABLE 5
Cross-coupling catalysis data

| | Grignard, | | | Cat. | |
|---|---|---|---|---|---|
| | RMgBr | | | loading | Yield |
| Trial | R= | Alkyl halide | Cat. | (%) | (%) |
| 1 | p-tolyl | bromocyclohexane | FeCl[O₂NN']^{ClClPy} | 5 | 28 |
| 2 | p-tolyl | bromocyclohexane | FeCl[O₂NN']^{ClClPy} | 10 | 31 |
| 3 | p-tolyl | bromocyclohexane | FeCl[O₂NN']^{ClClPy} | 15 | 25 |
| 4 | p-tolyl | bromocyclohexane | FeCl[O₂NN']^{ClClPy} | 20 | 23 |
| 5 | p-tolyl | bromocyclohexane | FeCl[O₂NN']^{FFPy} | 5 | 29 |
| 6 | p-tolyl | bromocyclohexane | FeCl[O₂NN']^{ClClNMe2} | 5 | 47 |
| 7 | p-tolyl | bromocyclohexane | FeBr[O₂NN']^{ClClNMe2} | 5 | 57 |
| 8 | Allyl | bromocyclohexane | FeCl[O₂NN']^{ClClPy} | 5 | 3 |
| 9 | Allyl | chlorocyclohexane | FeCl[O₂NN']^{ClClPy} | 5 | 5 |
| 10 | Allyl | iodocyclohexane | FeCl[O₂NN']^{ClClPy} | 5 | 7 |
| 11 | Allyl | 2-bromobutane | FeCl[O₂NN']^{ClClPy} | 5 | 5 |
| 12 | Allyl | 2-bromopentane | FeCl[O₂NN']^{ClClPy} | 5 | 2 |
| 13 | Allyl | benzylbromide | FeCl[O₂NN']^{ClClPy} | 5 | 13 |

### Example 5

### Controlled Radical Polymerization General Procedure

All manipulations and handling of ligands and iron complexes were performed in air. Cross-coupling experiments were done under nitrogen using Schlenk technique. Reagents were purchased and used without further purification.

Monomers styrene, methyl methacrylate, methyl acrylate and vinyl acetate were purchased from Aldrich Chemical Co. and dried by stirring over calcium hydride for 24 hours, before being vacuum transferred or distilled, degassed and stored at −35° C. under inert atmosphere. Azobis(isobutyronitrile) (AIBN) was purchased from Aldrich, recrystallized from methanol prior to use, and then stored at −35° C. under inert atmosphere.

All experiments involving moisture and air sensitive compounds were performed under a nitrogen atmosphere using an MBraun LABmaster sp glovebox system equipped with a −35° C. freezer and [H₂O] and [O₂] analyzers or using standard Schlenk techniques. Gel permeation chromatography (GPC) was carried out in THF (flow rate: 1 mL min⁻¹) at 50° C. with a Polymer Labs PL-GPC 50 Plus integrated GPC system using two 300×7.8 mm Jordi gel DVB mixed bed columns. For PS and PVAc, polystyrene standards were used for calibration and corrected for PVAc against parameters for low molecular weight vinyl acetate. For PMMA and PMA, poly(methyl methacrylate) standards were used.

¹H-NMR and 2-D spectra were recorded at 298 K with a Bruker Avance Spectrometer (300 MHz) in CDCl₃. Thermogravimetric analysis (TGA) was carried out using a TA Instruments TGA Q500 under an inert nitrogen atmosphere, with a flow rate of 60 mL min⁻¹ and a heating rate of 10° C. min⁻¹. Differential scanning calorimetry (DSC) was carried out using a TA Instruments DSC Q100 with a flow rate of 50 mL min⁻¹ and a heating rate of 5° C. min⁻¹. UV-vis data was collected using a Cary 100 instrument at 298 K with 1 cm pathlength.

Polydispersity Index (PDI)

The terms “dispersity” and “polydispersity” describe the dispersions of distributions of molar masses (or relative molecular masses, or molecular weights) and degrees of polymerization in polymeric systems. (INTERNATIONAL UNION OF PURE AND APPLIED CHEMISTRY—Dispersity in polymer science IUPAC Recommendations 2009; Pure Appl. Chem., Vol. 81, No. 2, pp. 351-353, 2009)

Polydispersity is measured by use of size exclusion chromatography, providing a distribution of molecular weights (Mₙ). Molecular weights are measured versus styrene standards and corrected (M_{n,corr}) for changes in monomer elution times. The ratio of Mₙ over M_{w} is the polydispersity (PDI). Other evidence of control includes a correlation between the observed molecular weight (Mₙ or M_{n,corr}) and the theoretical molecular weight M_{n,th} which is determined from the % conversion x the number of monomer units per chain x the molecular weight of the monomer.

Good control over the radical polymerization is evidenced by polymers with PDIs of <1.4 where good control refers to an ability to prepare polymers of narrow molecular weight distributions and predictable molecular weights. Excellent levels of control, approaching that of a living polymerization with an idealized polydispersity of 1.0, are evidenced by PDIs of <1.2.

General Procedure for Controlled Radical Polymerization

Monomer, catalyst and initiator in the ratio of about 100:1:0.6 were placed in an ampoule under inert atmosphere. The ampoule was stirred in a preheated oil-bath at 120° C. for the required length of time, then removed from the heat and cooled quickly under running water. Work-up procedures were dependent on the monomer: poly(styrene), poly(methyl methacrylate) and poly(methacrylate) samples were dissolved in 5 mL of THF and precipitated into 150 mL of acidified methanol (1% HCl). Monomer conversion for these reactions was determined by ¹H NMR spectroscopic analysis of crude samples, by comparing the integration of the polymer versus monomer resonances. For poly(vinyl acetate), excess monomer was removed under reduced pressure, the samples were dried to constant mass and then weighed to determine monomer conversion gravimetrically.

Polymerization of styrene with FeCl[O₂NN′]^{ClClNMe2}

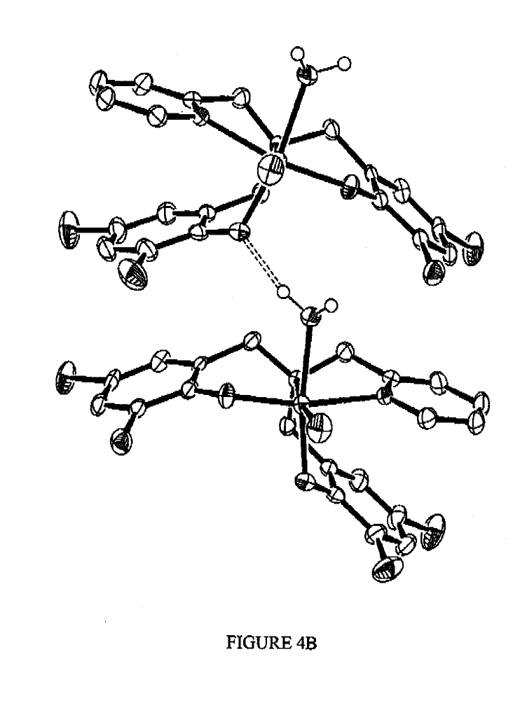

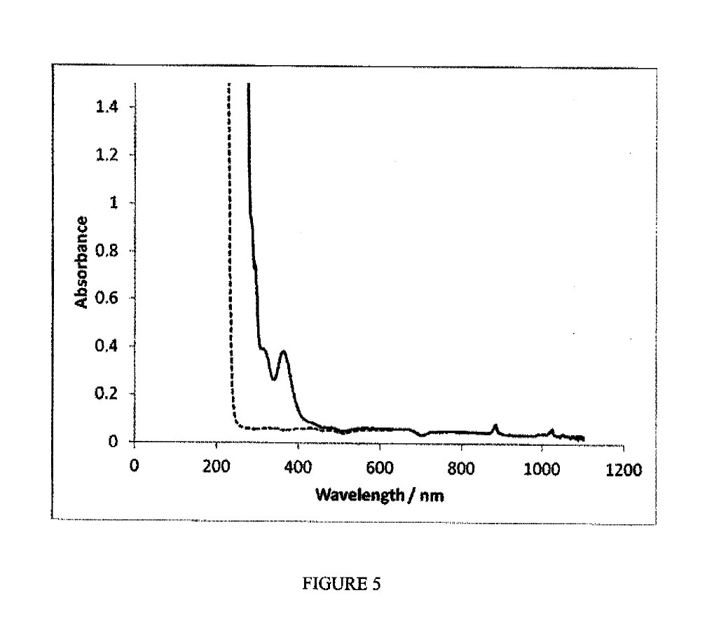

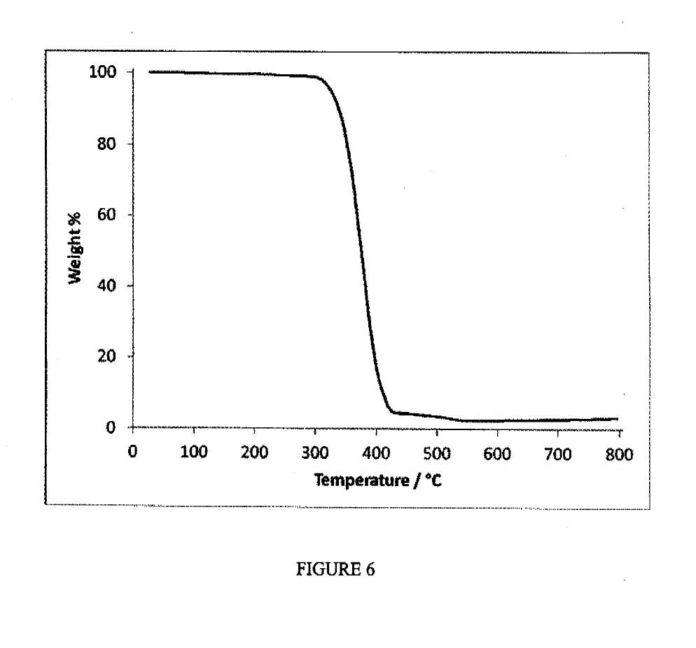

FeCl[O₂NN′]^{ClClNMe2} (0.05 g, 0.1 mmol), AIBN (0.01 g, 0.06 mmol) and styrene (1.0 g, 10 mmol) were added to an ampoule containing a micro-stirrer bar under inert atmosphere, which was then sealed and heated at 120° C. with stirring for 7.5 h. ¹H NMR spectroscopic analysis of the crude residue indicated 90% monomer conversion, with GPC analysis of the crude material giving an Mₙ of 9933 and a PDI of 1.17. Precipitation into acidified methanol gave white poly(styrene), with Mₙ=10941 and PDI=1.16. The UV-Vis spectrum of the worked-up polystyrene product LA-216 is shown in FIG. 5. The Thermogravimetric analysis (TGA) and Differential scanning calorimetry (DSC) traces of the worked-up polystyrene product LA-216 is shown in FIGS. 6 and 7, respectively.

ATRP with Various Monomers

The polymerization procedure using various monomers was performed according to the general conditions described above. The catalysts employed in each polymerization are described in the general structures IIa and IIb as described earlier. Data for each set of reactions is provided in Tables 6-11.

TABLE 6
Vinyl acetate Monomer ATRP

| Rxn | | % | | | | | |
|---|---|---|---|---|---|---|---|
| (LA-) | Complex | conv. | Mₙ | M_{n,corr.} | M_{n,th} | M_{w} | PDI |
| 138 | {FeCl[O₂N]^{BuMePr}}₂ | 13 | 98613 | 69029 | 970 | 264635 | 2.68 |
| 170 | FeCl[O₂NO]^{BuMeFurf} | 28 | 5423 | 3796 | 1999 | 277723 | 5.11 |
| | | | | | | | (bimodal) |
| 140 | FeBr[O₂NO]^{BuMeFurf} | 10 | 47966 | 33576 | 746 | 105891 | 2.21 |
| 172 | FeBr[O₂NO]^{BuBuFurf} | 21 | 108620 | 76034 | 1567 | 286975 | 2.64 |
| 176 | FeBr[O₂NO]^{BuBuMeth} | 16 | 23597 | 16518 | 1194 | 53109 | 2.25 |
| 178 | FeBr[O₂NN′]^{ClClNMe2} | 10 | 19795 | 13857 | 746 | 59040 | 2.93 |
| 180 | FeCl[O₂NN′]^{ClClNMe2} | 10 | 77667 | 54367 | 746 | 189003 | 2.43 |
| 182 | FeBr[O₂NO]^{ClClMeth} | 9 | 14648 | 10254 | 672 | 93474 | 6.38 |
| 184 | FeCl[O₂NO]^{ClClFurf} | 7 | 68629 | 48040 | 522 | 176625 | 2.57 |
| 186 | FeCl[O₂NN′]^{ClClPy} | ca 8 | 95750 | 67025 | 597 | 182639 | 1.91 |
| 188 | FeBr[O₂NN′]^{ClClPy}(H₂O) | 11 | 79858 | 55901 | 821 | 145430 | 1.82 |

Bulk vinyl acetate polymerizations, initiated with AIBN at 120° C. for 6 h with a complex:initiator:monomer ratio of 1:0.6:100.
M_{n,th} assumes 2 radicals per AIBN.
M_{n,corr.} uses conversion factor of 0.7.

TABLE 7
Styrene, initial screening with long reaction times.

| Rxn | | | | | | |
|---|---|---|---|---|---|---|
| (LA-) | Complex | % conv. | Mₙ | M_{n,th} | M_{w} | PDI |
| 137 | {FeCl[O₂N]^{BuMePr}}₂ | 54 | 7543 | 4667 | 10138 | 1.34 |
| 168^{a} | {FeCl[O₂N]^{BuMePr}}₂ | 85 | 13665 | 7346 | 18864 | 1.38 (low |
| | | | | | | MW tailing) |
| 174^{b} | {FeCl[O₂N]^{BuMePr}}₂ | 85 | 6947 | 7346 | 9215 | 1.33 |
| 169 | FeCl[O₂NO]^{BuMeFurf} | 79 | 12916 | 6900 | 25972 | 2.01 |
| 139 | FeBr[O₂NO]^{BuMeFurf} | 69 | 15196 | 5963 | 26034 | 1.71 (low |
| | | | | | | MW tailing) |
| 171 | FeBr[O₂NO]^{BuBuFurf} | 86 | 13905 | 7433 | 33935 | 2.44 |
| | | | | | | (bimodal) |
| 175 | FeBr[O₂NO]^{BuBuMeth} | 89 | 12669 | 7692 | 25082 | 1.98 |
| | | | | | | (bimodal) |
| 177 | FeBr[O₂NN′]^{ClClNMe2} | 87 | 9174 | 7519 | 17624 | 1.92 |
| 179 | FeCl[O₂NN′]^{ClClNMe2} | ca 100 | 13261 | 8643 | 17110 | 1.29 |
| 181 | FeBr[O₂NO]^{ClClMeth} | 89 | 3722 | 7692 | 4426 | 1.19 |
| 183 | FeCl[O₂NO]^{ClClFurf} | 87 | 7197 | 7519 | 9033 | 1.26 |
| 185 | FeCl[O₂NN′]^{ClClPy} | 91 | 9145 | 7865 | 10771 | 1.18 |
| 187 | FeBr[O₂NN′]^{ClClPy}(H₂O) | 59 | 6004 | 5099 | 6582 | 1.10 |

Bulk styrene polymerizations, initiated with AIBN at 120° C. for 6 h with a complex:initiator:monomer ratio of 1:0.6:100. M_{n,th} assumes 2 radicals per AIBN.
^{a}Run time = 21 h.
^{b}Run time = 3.5 h.

TABLE 8
Styrene, short reaction times.

| Rxn | | % | | | | |
|---|---|---|---|---|---|---|
| (JM-) | Complex | conv. | Mₙ | M_{n,th} | M_{w} | PDI |
| 28 | FeBr[O₂NO]^{BuMeFurf} | 48 | 10541 | 4166 | 15460 | 1.47 |
| 29 | FeBr[O₂NO]^{BuBuMeth} | 50 | 11461 | 4340 | 18229 | 1.64 |
| 32 | FeBr[O₂NN′]^{ClClNMe2} | 46 | 5633 | 3992 | 6281 | 1.12 |
| 34 | FeCl[O₂NN′]^{ClClNMe2} | 40 | 4962 | 3472 | 5610 | 1.13 |
| 35 | FeBr[O₂NO]^{ClClMeth} | 47 | 4927 | 4079 | 6344 | 1.14 |
| 90 | FeCl[O₂NO]^{ClClFurf} | 54 | 7815 | 4687 | 9904 | 1.27 |
| 31 | FeCl[O₂NN′]^{ClClPy} | 60 | 8506 | 5208 | 9451 | 1.11 |
| 30 | FeBr[O₂NN′]^{ClClPy}(H₂O) | 37 | 4650 | 3298 | 5414 | 1.16 |

Bulk styrene polymerizations, initiated with AIBN at 120° C. for 1 h with a complex:initiator:monomer ratio of 1:0.6:100.
M_{n,th} assumes 2 radicals per AIBN.

TABLE 9
Styrene, with 200:1 monomer:initiator ratios.

| Rxn (JM) | Complex | % conv. | Mₙ | M_{n,th} | M_{w} | PDI |
|---|---|---|---|---|---|---|
| 6 | FeBr[O₂NO]^{BuMeFurf} | 48 | 17828 | 8245 | 35656 | 1.47 |
| 7 | FeBr[O₂NO]^{BuBuMeth} | 70 | 19695 | 12151 | 49438 | 2.50 |
| 13 | FeBr[O₂NN′]^{ClClNMe2} | 28 | 7402 | 4860 | 8518 | 1.15 |
| 1 | FeCl[O₂NN′]^{ClClNMe2} | 51 | 11654 | 8853 | 13884 | 1.20 |
| 5 | FeBr[O₂NO]^{ClClMeth} | 49 | 7772 | 8419 | 9875 | 1.30 |
| 4 | FeCl[O₂NO]^{ClClFurf} | 57 | 10283 | 9842 | 13375 | 1.30 |
| 2 | FeCl[O₂NN′]^{ClClPy} | 60 | 14997 | 10415 | 20186 | 1.35 |
| 8 | FeBr[O₂NN′]^{ClClPy}(H₂O) | 43 | 7785 | 7464 | 9515 | 1.22 |

Bulk styrene polymerizations, initiated with AIBN at 120° C. for 1 h with a complex:initiator:monomer ratio of 1:0.6:200.
M_{n,th} assumes 2 radicals per AIBN.

TABLE 10
Methyl methacrylate in bulk. Conversions could not be measured
as reactions proceeded too quickly to remove samples.

| Rxn | | % | | | | |
|---|---|---|---|---|---|---|
| (JM-) | Complex | conv. | Mₙ | M_{n,th} | M_{w} | PDI |
| 38 | FeBr[O₂NO]^{BuMeFurf} | >99 | 14080 | 8343 | 21559 | 1.53 |
| 58 | FeBr[O₂NO]^{BuBuMeth} | >99 | 12809 | 8343 | 19174 | 1.50 |
| 52 | FeBr[O₂NN′]^{ClClNMe2} | >99 | 10473 | 8343 | 12646 | 1.21 |
| 53 | FeCl[O₂NN′]^{ClClNMe2} | >99 | 8289 | 8343 | 10079 | 1.22 |
| 48 | FeBr[O₂NO]^{ClClMeth} | >99 | 7459 | 8343 | 8634 | 1.14 |
| 55 | FeCl[O₂NO]^{ClClFurf} | >99 | 8776 | 8343 | 11946 | 1.36 |
| 40 | FeCl[O₂NN′]^{ClClPy} | >99 | 10193 | 8343 | 14556 | 1.33 |
| 45 | FeBr[O₂NN′]^{ClClPy}(H₂O) | >99 | 8366 | 8343 | 9950 | 1.19 |

Bulk MMA polymerizations, initiated with AIBN at 120° C. for 1 h with a complex:initiator:monomer ratio of 1:0.6:100.
M_{n,th} assumes 2 radicals per AIBN.

TABLE 11
Methyl methacrylate in toluene. Dilution gives slower kinetics.

| Rxn | | % | | | | |
|---|---|---|---|---|---|---|
| (JM-) | Complex | conv. | Mₙ | M_{n,th} | M_{w} | PDI |
| 20 | FeBr[O₂NO]^{BuMeFurf} | 57 | 11547 | 4756 | 16555 | 1.43 |
| 21 | FeBr[O₂NO]^{BuBuMeth} | 51 | 12535 | 4225 | 18811 | 1.50 |
| 24 | FeBr[O₂NN′]^{ClClNMe2} | 71 | 10908 | 5924 | 15302 | 1.40 |
| 19 | FeCl[O₂NN′]^{ClClNMe2} | 51 | 8694 | 4255 | 10343 | 1.19 |
| 25 | FeBr[O₂NO]^{ClClMeth} | 42 | 6627 | 3502 | 8106 | 1.22 |
| 18 | FeCl[O₂NO]^{ClClFurf} | 49 | 6849 | 4088 | 9376 | 1.29 |
| 22 | FeCl[O₂NN′]^{ClClPy} | 40 | 7574 | 3337 | 10154 | 1.34 |
| 23 | FeBr[O₂NN′]^{ClClPy}(H₂O) | 43 | 7528 | 3588 | 8765 | 1.16 |

MMA polymerizations, 1:1 w/w toluene, initiated with AIBN at 120° C. for 1 h with a complex:initiator:monomer ratio of 1:0.6:100.
M_{n,th} assumes 2 radicals per AIBN.

The described complexes were found to capably control the polymerization of styrene, methyl methacrylate and methacrylate. The rates of these systems are comparable to those observed for copper complexes, but the polymers produced were white and the remaining iron is benign. Extensive screening and kinetic plots confirmed the complexes' activity as ATRP mediators.

As discussed above, good control was shown for polymers with polydispersity indeces (PDI) of <1.4. The best iron complexes reported in the literature reach polydispersities (PDI) of 1.2, a value considered excellent. Using the present amine-bis(phenolate) iron catalysts it was possible to control down to 1.10 for styrene and 1.16 for methyl methacrylate. These represent remarkable levels of control.

### Example 6

### General Polymerization Procedure for Styrene Kinetics

Monomer, catalyst and initiator in the ratio 100:1:0.6 were placed in a schlenk flask under inert atmosphere and sealed with a subaseal. The schlenk was placed in an oil-bath preheated to 120° C., at which point timing commenced. Samples were removed from the schlenk via degassed syringe at designated intervals and quenched with CDCl₃. Analysis of the crude samples by ¹H NMR spectroscopy gave the monomer conversion, while GPC analysis gave the molecular weights and PDIs of the samples. The results are shown in Table 12 below.

TABLE 12
Kinetic studies using FeCl[O₂NN']^{ClClNMe2} (styrene).

| Time/ | | | | | |
|---|---|---|---|---|---|
| min | % conv. | ln[M]₀/[M]ₜ | Mₙ | M_{n, th} | PDI |
| 10 | 30 | 0.356675 | 2981 | 2565.78 | 1.31 |
| 20 | 43 | 0.562119 | 3706 | 3677.618 | 1.28 |
| 30 | 49 | 0.673345 | 4379 | 4190.774 | 1.22 |
| 45 | 54 | 0.776529 | 4874 | 4618.404 | 1.18 |
| 60 | 59 | 0.891598 | 5163 | 5046.034 | 1.18 |
| 75 | 63 | 0.994252 | 5423 | 5388.138 | 1.17 |
| 90 | 67 | 1.108663 | 5545 | 5730.242 | 1.17 |
| 105 | 71 | 1.237874 | 6104 | 6072.346 | 1.17 |
| 120 | 73 | 1.309333 | 6280 | 6243.398 | 1.16 |
| 180 | 81 | 1.660731 | 6438 | 6927.606 | 1.16 |
| 255 | 89 | 2.207275 | 6836 | 7611.814 | 1.16 |

Bulk styrene polymerization, initiated with AIBN at 120° C. for 1 h with a complex:initiator:monomer ratio of 1:0.6:100.
M_{n,th} assumes 2 radicals per AIBN.

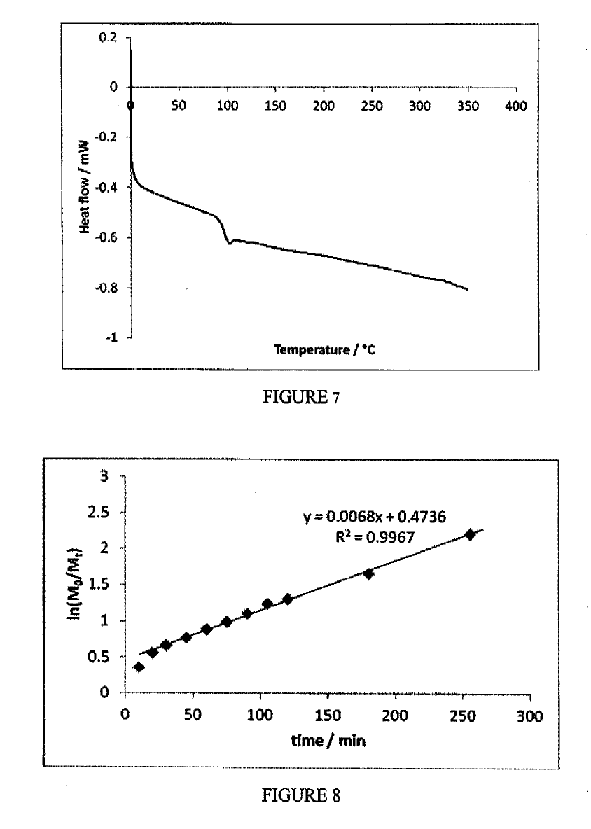

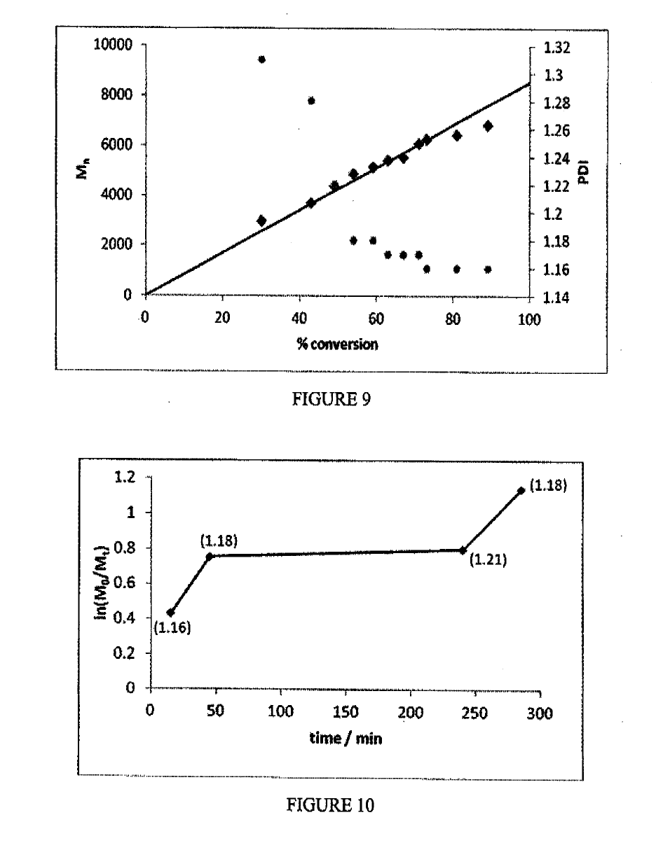

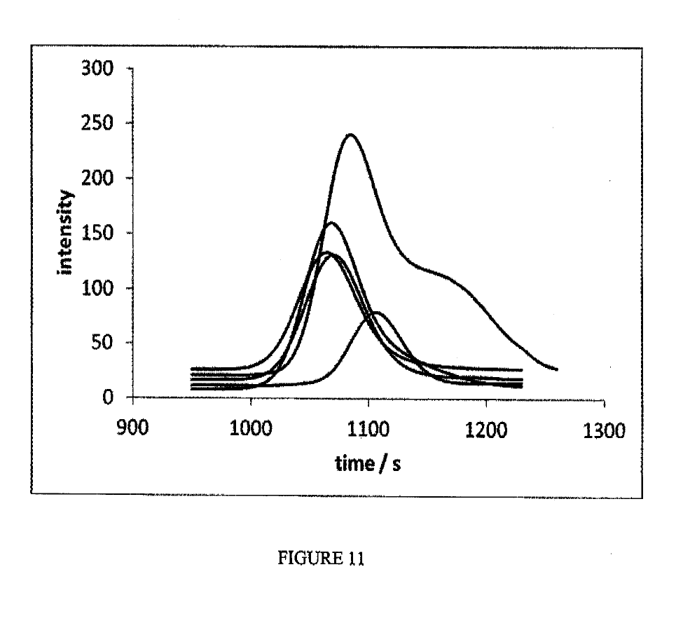

FIG. 8 is a Plot of ln [M]₀/[M], versus time for bulk styrene polymerization at 120° C. using FeCl[O₂NN′]^{ClClNMe2} and AIBN, with a complex:initiator:monomer ratio of 1:0.6:100.] FIG. 9 is a plot of molecular weight (♦) and PDI () versus conversion for bulk styrene polymerization at 120° C. using FeCl[O₂NN′]^{ClClNMe2} and AIBN, with a complex:initiator:monomer ratio of 1:0.6:100.] FIG. 10 is a Stop-start plot of ln [M]₀/[M]ₜ versus time for bulk styrene polymerization at 120° C. using FeCl[O₂NN′]^{ClClNMe2} and AIBN, with a complex:initiator:monomer ratio of 1:0.6:100. PDI values shown in parentheses. The concentration of AIBN initiator was varied and the results to PDI are reported as follows.

TABLE 13
Varying AIBN concentration.

| Reaction | | | | | |
|---|---|---|---|---|---|
| (LA-) | AIBN eq. | % conv. | Mₙ | M_{n, th} | PDI |
| 244 | 1.5 | 78 | 4940 | 2668 | 1.29 |
| 245 | 0.3 | 26 | 3611 | 4342 | 1.11 |
| 246 | 0.5 | 56 | 5893 | 5820 | 1.16 |
| 247 | 6.0 | 92 | 2508 | 795 | 1.63* |
| 248 | 0.6 | 63 | 6121 | 5445 | 1.20 |

100:1 monomer:complex ratio, 120° C. 1 h.
*indicates bimodal distribution.

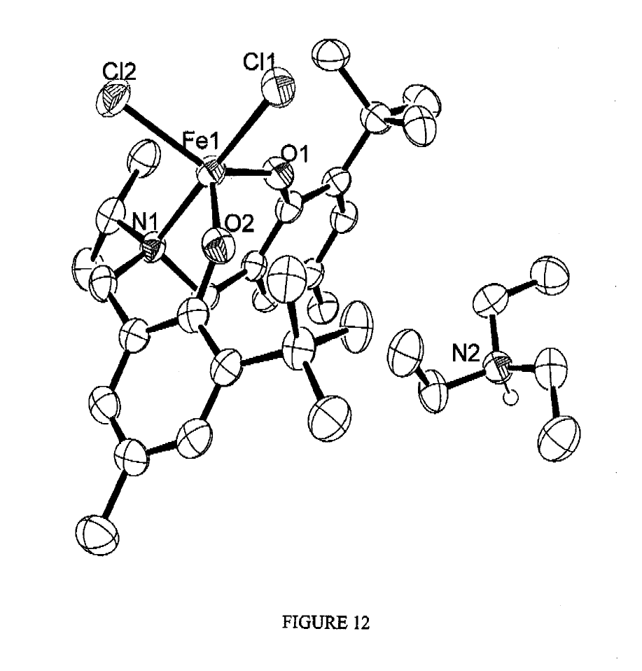

FIG. 11 graphically depicts the GPC traces for bulk styrene polymerization at 120° C. using FeCl[O₂NN′]^{ClClNMe2} and AIBN, with a complex:monomer ratio of 1:100. From highest to lowest intensity, the lines depict the reaction speed of reactions having the following equivalents of AIBN: 6 eq, 1.5 eq, 0.6 eq, 0.5 eq, and 0.3 eq.

### Example 7

### Polymerization of Styrene and MMA

Mechanistic studies were performed for the present catalysts in CRP reactions. CRP offers polymer chemists and engineers the ability to alter polymer macrostructure and create a unique array of materials with high functional group tolerance and defined molecular weights. Metal-mediated methods such as (reverse) atom transfer radical polymerization ((R)ATRP) (di Lena, F.; Matyjaszewski, K. Prog. Polym. Sci. 2010, 35, 959) and organometallic mediated radical polymerization (OMRP), as shown Scheme 6, are especially useful as tuning the supporting ligand framework in a metal complex can expand the monomer scope and open up new applications. (Matyjaszewski, K.; Tsarevsky, N. V. Nat. Chem. 2009, 1, 276)

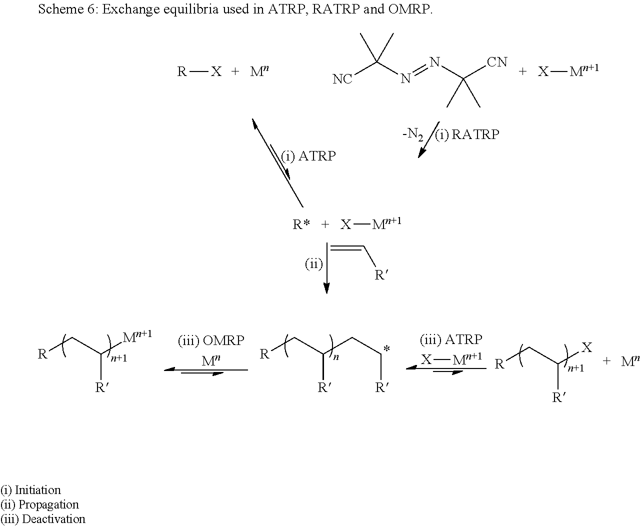

The following catalysts were prepared for this polymerization study:

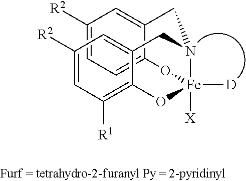

| Compound | R¹ | R² | D | X |
|---|---|---|---|---|
| 21 | tBu | Me | CH₂Furf | Cl |
| 22 | tBu | Me | CH₂Furf | Br |
| 23 | tBu | tBu | CH₂Furf | Br |
| 24 | tBu | tBu | (CH₂)₂OMe | Br |
| 25 | Cl | Cl | (CH₂)₂NMe₂ | Cl |
| 26 | Cl | Cl | (CH₂)₂NMe₂ | Br |
| 27 | Cl | Cl | CH₂Furf | Cl |
| 28 | Cl | Cl | CH₂Py | Cl |
| 29 | Cl | Cl | CH₂Py | Br |
| 31 | Cl | Cl | (CH₂)₂OMe | Br |

Initial studies utilized ^{Cl,Cl,NMe2}[O₂NN′]FeCl, which proved to be among the fastest iron-based catalysts for the CRP of styrene reported, with a k_{obs.} of 1.02 h⁻¹ (cf salicylaldiminato iron complexes (O'Reilly, R. K. J. Am. Chem. Soc. 2003, 125, 8450) with k_{obs.}=0.39-0.49 h⁻¹ and α-diimine iron complexes (Allan, L. E. N. Inorg. Chem. 2007, 46, 8963), with k_{obs.}=0.01-0.72 h⁻¹). The linear semilogarithmic plot of ln [M]₀/[M]ₜ versus time and the linear increase of molecular weight with conversion, in conjunction with the narrow PDIs, illustrated the excellent control imparted by this complex. However, in repeated kinetic experiments molecular weights were observed to be somewhat higher than the theoretical values. This can be attributed to the number of growing radical chains being lower than expected, resulting in an effective increase in the monomer concentration. Polymerization was very rapid initially (reaching 32% conversion in 10 minutes) before a constant radical concentration was established and linear behaviour observed. End-group analysis of low molecular weight crude polymer samples by ¹H NMR spectroscopy suggests that the polymerization mechanism was not simply RATRP, as only 30-35% of the chains are chlorine-terminated. No evidence of olefin end-groups was observed and the success of a start-stop reaction implied that the other polymerization pathway also operates though a controlled radical mechanism, potentially OMRP.

Mechanistic Studies with Styrene Monomer

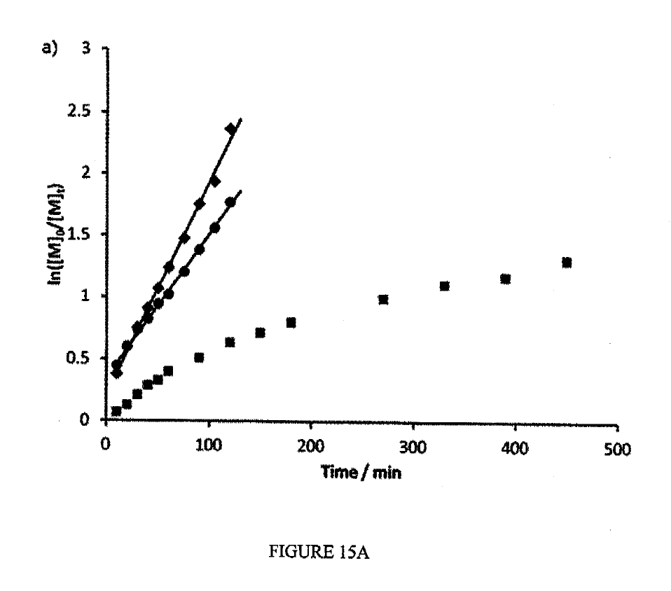

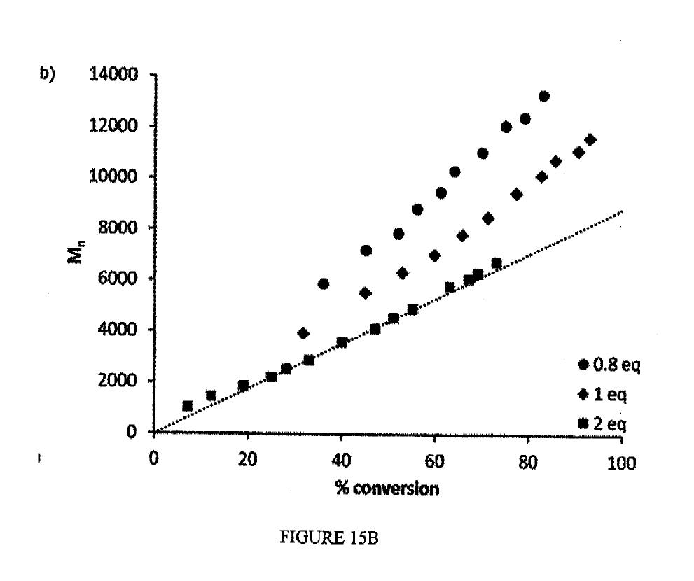

The effect of changing the concentration of both the initiator (Table 14, FIG. 14) and the catalyst (FIGS. 15A and 15B) was investigated. FIG. 14 shows GPC traces for bulk styrene polymerizations using ^{Cl,Cl,NMe2}[O₂NN′]FeCl. [Fe]:[St] ratio 1:100, 120° C., 1 h. FIGS. 15A and 15B show a plot of ln([M]₀/[M]ₜ) versus time for bulk styrene polymerizations and molecular weight versus conversion plots for various equivalents of ^{Cl,Cl,NMe2}[O₂NN′]FeCl, respectively. The dashed line indicates theoretical molecular weights. Use of 6 equivalents of AIBN led to a loss in control, where high conversions were rapidly achieved as a result of the high radical concentrations. Molecular weights were much higher than the theoretical values and broad, bimodal PDIs were observed. The excess radicals cannot be deactivated by the catalyst fast enough and so bimolecular coupling and other termination reactions occur. The use of 1.5 eq. of AIBN resulted in a slight loss of control, with PDIs broadening to 1.29 and molecular weights which were higher than theoretical values. However, the catalyst still imparts reasonable control over the polymerization, even at this significantly elevated radical concentration. This suggests that multiple trapping routes are available to the propagating chains and that very fast chain exchange occurs. At radical concentrations below our standard 0.6 eq. of AIBN, excellent control is observed, although the polymerizations are slower. Using 0.5 eq. of AIBN instead of 0.6 decreased the conversion from 63% in 1 h to 56%, but control over both the molecular weight and PDI was improved. With 0.3 eq. of AIBN, the catalyst is in excess and deactivation of the propagating radicals is favored. The polymerization is significantly slower and this results in excellent PDIs of 1.11.

TABLE 14
Effect of AIBN concentration on styrene polymerization.

| AIBN eq. | % conv. | M_{n, th} | Mₙ | PDI |
|---|---|---|---|---|
| 0.3 | 26 | 4542 | 3611 | 1.11 |
| 0.5 | 56 | 5820 | 5893 | 1.16 |
| 0.6 | 63 | 5445 | 6121 | 1.19 |
| 1.5 | 78 | 2668 | 4940 | 1.29 |
| 6.0 | 92 | 795 | 2508 | 1.63* |

[Fe]:[St] ratio 1:100, 120° C., 1 h, using ^{Cl,Cl,NMe2}[O₂NN']FeCl.
% conv. determined from ¹H NMR spectra of crude samples.
*indicates bimodal distribution.
M_{n,th} = [St]₀/2[AIBN]₀ × MW(St) × conversion.

Changing the concentration of catalyst also had a significant effect on the polymerization. Increasing the amount of trapping agent bad the expected effect of slowing the rate of polymerization, which significantly improved the PDIs. When 2 equivalents of catalyst were used, non-linear plots of ln([M]₀/[M]ₜ) were obtained (FIG. 15A) and the polymerization took 8 h to reach 50% conversion, albeit with PDIs which were below 1.16 throughout and <1.10 for the first 5 h. Molecular weights were in excellent agreement with theoretical values (FIG. 15B), indicating the exemplary control over the polymerization. A decrease in catalyst concentration to 0.8 equivalents also resulted in slower rates of polymerization when compared to the reaction using 1.0 equivalents (k_{obs.}=0.013 h⁻¹ for 0.8 eq., cf 0.017 h⁻¹ for 1.0 eq.), which can be attributed to the lower radical concentrations overall. It is worth noting that the initial rate (first 20 minutes) is faster when 0.8 eq. of catalyst is used, suggesting that increased amounts of catalyst reduce the uncontrolled events at the beginning of the polymerization. Molecular weights obtained using 0.8 eq. of catalyst were significantly higher than the theoretical values, deviating more severely than data obtained for 1.0 equivalents of catalyst. PDIs were also broader, decreasing from 1.27 at the beginning of the polymerization to 1.17 at the end.

Polymerization with MMA Monomer

The iron complexes 21-29 and 31 were screened for activity towards MMA, with the chloro-substituted complexes proving to be most efficient. It was surprising to find that the MMA solution polymerizations (monomer 1:1 w/w with toluene) were not significantly faster than the styrene reactions, despite the greater reactivity of MMA (Table 15). Bulk reactions resulted in broader PDIs and slightly increased conversions, but in all cases molecular weights were higher than the theoretical values calculated from the initial concentration of radical initiator. This indicates that fewer chains were growing than expected and is consistent with data obtained for the styrene polymerizations.

TABLE 15
Initial methyl methacrylate polymerization screening reactions

| Complex | % conv. | M_{n, th}^{b} | Mₙ^{c} | PDI^{c} |
|---|---|---|---|---|
| 21 | 57 | 4750 | 11570 | 1.43 |
| 22 | 51 | 4230 | 12540 | 1.50 |
| 25 | 40 | 3340 | 7570 | 1.24 |
| 25 | 48* | 4010 | 8120 | 1.28 |
| 26 | 67 | 5607 | 8272 | 1.34 |
| 28 | 51 | 4260 | 8700 | 1.19 |
| 23 | 71 | 5920 | 10900 | 1.40 |
| 24 | 42 | 3500 | 6630 | 1.22 |
| 27 | 49 | 4090 | 6850 | 1.29 |

[Fe]:[MMA]:[AIBN] ratio of 1:100:0.6 1 h at 120° C., solvent 1:1 (w/w) with monomer except for * which is bulk.
M_{n,th} = [MMA]₀/(2 × [AIBN]₀) × MMA molecular weight × conversion

Effect of Initiator Concentration

The effect of initiator concentration on the bulk MMA polymerizations was investigated using ^{Cl,Cl,NMe2}[O₂NN′]FeCl (Table 16). Results were significantly different to those obtained for styrene under identical conditions. A decrease in the initial AIBN concentration to 0.3 eq. resulted in little change to the PDI (PDI=1.28 and 1.29 for 0.6 and 0.3 equivalents of AIBN, respectively). No significant change in molecular weights was observed, although conversion decreased as a result of reduced radical concentrations. Increasing the AIBN concentration to 1.5 eq., resulted in largely unchanged PDIs, but conversion was considerably higher (88% vs. 48%) after 1 h, attributed to the higher number of radicals generated initially. Molecular weights were still much higher than expected if theoretical molecular weights are calculated from [AIBN]₀. Further increasing the AIBN concentration to 3 eq. resulted in improved control, illustrated by a decrease in PDI to 1.24. This was unexpected as the radical concentration is much higher than the catalyst concentration which should result in uncontrolled propagation, leading to the formation of uncontrolled polymer. Increasing the initial AIBN concentration to 6 eq. improves control further. The molecular weight of the polymer obtained was much higher than the theoretical values, as 6 eq. of AIBN leads to the formation of 12 radicals per metal centre. In reverse ATRP this is expected to lead to a very uncontrolled system, as there are too many propagating radicals to be controlled by the low catalyst concentration. This suggests that this iron system does not operate exclusively by reverse ATRP.

TABLE 16
Effect of AIBN concentration on MMA polymerization

| AIBN eq. | % conv. | M_{n, th} | Mₙ | PDI |
|---|---|---|---|---|
| 0.3 | 29 | 4840 | 9990 | 1.29 |
| 0.6 | 48 | 4010 | 8120 | 1.28 |
| 1.5 | 88 | 3000 | 7710 | 1.29 |
| 3 | 90 | 1500 | 5070 | 1.24 |
| 6 | 93 | 780 | 3780 | 1.18 |

[Fe]:[MMA] ratio 1:100, 120° C., 1 h, using ^{Cl,Cl,NMe2}[O₂NN']FeCl.
% conv. determined from ¹H NMR spectra of crude samples.
M_{n,th} = [MMA]₀/2[AIBN]₀ × MW(MMA) × conversion.

Effect of Temperature

As the polymerization of MMA with higher concentrations of radical initiator afforded better control, it was investigated whether slower generation of radicals would be advantageous. Bulk MMA polymerizations using AIBN as the initiator at 50° C. and 70° C. were carried out, but the data obtained show that the system did not operate well with a slower generation of radicals (Table 17). Conversions were quite low at 50° C., which is as expected, as much fewer radicals are generated. However, PDIs were much broader (1.39-1.49), indicating inefficient exchange as well as new chains starting as the polymerization proceeds. This leads to polymers of varying lengths and broad PDIs. When increasing the temperature up to 70° C., conversion was much higher but exchange was still inefficient, resulting in a bimodal distribution. AIBN was not efficient at these lower temperatures and other radical initiators with lower 10 hour half-life decomposition temperature, V-65 and V-70 (Table 17), were investigated.

TABLE 17
Effect of lower temperatures on MMA
polymerization using AIBN, V-65 and V-70.

| | In. | | | | | | |
|---|---|---|---|---|---|---|---|
| Initiator | eq. | Temp./° C. | Time/h | % conv. | M_{n,th} | Mₙ | PDI |
| AIBN | 3 | 50 | 6 | 22 | 350 | 15921 | 1.39 |
| AIBN | 6 | 50 | 6 | 29 | 242 | 14847 | 1.49 |
| AIBN | 6 | 70 | 1.5 | 70 | 584 | 9519 | 1.28* |
| AIBN | 6 | 120 | 1 | 93 | 780 | 3780 | 1.18 |
| V-65 | 0.6 | 90 | 1 | 24 | 2070 | 6610 | 1.24 |
| V-65 | 0.6 | 100 | 1 | 25 | 2120 | 5932 | 1.23 |
| V-65 | 0.6 | 120 | 1 | 35 | 2920 | 8570 | 1.24 |
| V-70 | 0.6 | 80 | 2 | 88 | 7375 | 10530 | 1.78 |
| V-70 | 6 | 80 | 0.33 | 86 | 721 | 9764 | 1.47 |
| V-70 | 6 | 65 | 6 | 44 | 369 | 11470 | 1.45 |

[Fe]:[MMA] ratio 1:100, using^{Cl,Cl,NMe2}[O₂NN′]FeCl.
% conv. determined from ¹H NMR spectra of crude samples.
*indicates bimodal distribution.
M_{n,th} = [MMA]₀/2[In.]₀ × MW(MMA) × conversion.

From Table 17, it can be seen that polymerization was efficiently initiated with V-65. Interestingly, the PDIs remained constant even when decreasing the temperature to 90° C., indicating that initiation was efficient at these lower temperatures. However, conversions were considerably lower, reaching only 35% conversion after 1 h at 120° C., compared to 51% in an equivalent reaction initiated by AIBN. At lower temperatures, conversion was decreased further to ca 25%. Molecular weights remained higher than theoretical values at all temperatures. In contrast, reactions initiated by V-70 were not well controlled. PDIs were broad, only decreasing to 1.45 when 6 eq. of initiator was used at 65° C. Theoretical molecular weights were calculated based on the initial concentration of the radical initiator, but did not correlate well with actual molecular weights. Basing theoretical molecular weights on the monomer to catalyst ratio (since not all radicals were initiating polymer chains), as in a DT-OMRP polymerization, yielded better correlation between theoretical and observed molecular weights.

Effect of Monomer Concentration

Changing the monomer to solvent ratio was investigated to evaluate the effect this would have on control over the polymerization (Table 18). As expected, these solution polymerizations were still slower than the bulk reactions and less controlled than the more dilute solution polymerizations. Molecular weights were still considerably higher than theoretical values and PDIs increased slightly as the monomer concentration (and thus effective radical concentration) increased.

TABLE 18
Effect of dilution on MMA polymerization

| [MMA]:[tol] | AIBN eq. | Time/h | % conv. | M_{n,th} | Mₙ | PDI |
|---|---|---|---|---|---|---|
| 3:1 | 0.6 | 1 | 28 | 1750 | 6080 | 1.27 |
| 3:1 | 0.6 | 4 | 70 | 4380 | 10390 | 1.28 |
| 4:1 | 0.6 | 1 | 26 | 2170 | 8420 | 1.32 |

[Fe]:[MMA] ratio 1:100, using^{Cl,Cl,NMe2}[O₂NN′]FeCl.
% conv. determined from ¹H NMR spectra of crude samples.
M_{n,th} = [MMA]₀/2[AIBN]₀ × MW(MMA) × conversion.

Solution polymerization of MMA at higher radical concentrations and higher monomer concentrations (Table 19) was also investigated. A 1:1 monomer to solvent ratio, with 3 or 6 eq. of AIBN yielded the best control achieved over MMA polymerization, with PDIs of 1.14. However, control was decreased when the amount of monomer added was increased. When increasing from 100 to 200 equivalents of MMA, PDIs increased from 1.24 to 1.31. The molecular weight did increase but values were still considerably higher than the theoretical values. When increasing from 100 to 500 equivalents, control was significantly decreased, with PDIs of >1.45 although the molecular weights increased as expected.

TABLE 19
Solution MMA polymerizations with higher
AIBN and monomer concentrations.

| MMA eq. | [MMA]:[tol] | AIBN eq. | % conv. | M_{n,th} | Mₙ | PDI |
|---|---|---|---|---|---|---|
| 50 | 1:1 | 3 | 90 | 750 | 3570 | 1.14 |
| 50 | 1:1 | 6 | 92 | 380 | 3790 | 1.14 |
| 100 | 1:1 | 3 | 90 | 1500 | 5070 | 1.24 |
| 200 | 1:1 | 3 | 81 | 2700 | 8600 | 1.31 |
| 200 | 1:2 | 3 | 83 | 2770 | 11060 | 1.29 |
| 500 | 1:1 | 3 | 56 | 4670 | 16770 | 1.45 |
| 500 | 1:5 | 3 | 78 | 5420 | 20070 | 1.49 |

Solution MMA polymerizations, using^{Cl,Cl,NMe2}[O₂NN′]FeCl.
% conv. determined from ¹H NMR spectra of crude samples.
M_{n,th} = [MMA]₀/2[AIBN]₀ × MW(MMA) × conversion.

Polymerization Under DT Conditions

OMRP can occur via two methods, reversible termination OMRP where the metal complex reversibly caps the propagating chain (as shown in Scheme 6), or degenerative transfer OMRP (DT-OMRP) which is a thermodynamically neutral bimolecular exchange between a low concentration of growing radical chains and a dormant species. DT-OMRP requires a constant influx of radicals throughout the polymerization, whereas RT-OMRP typically requires an external initiator to start the polymerization, but the homolytic cleavage of the M-R dormant species then provides the only source of radicals. DT-OMRP conditions were investigated by combining an initiator which would decompose quickly at the polymerization temperature (V-70) with an initiator operating at its 10-hour half life temperature, AIBN. The results are shown in Table 20, for both styrene and MMA.

TABLE 20
Effect of mixed initiators on styrene and MMA polymerizations.

| Monomer | Time/h | % conv. | M_{n, th} | Mₙ | PDI |
|---|---|---|---|---|---|
| St | 1 | 55 | 6330 | 4132 | 2.35 |
| St | 3 | 91 | 9600 | 8146 | 2.61 |
| MMA | 4 | 95 | 10164 | 7169 | 1.50 |
| MMA | 6 | 97 | 9839 | 4529 | 2.03 |

Bulk St polymerizations, MMA 1:1 v/v with toluene, with a complex:V-70:AIBN:monomer ratio of 1:0.6:5:100.
M_{n,th} = [M]₀/[cat]₀ × MW(monomer) × conversion.

It was found that the present complexes containing electron-withdrawing substituents on the aromatic rings were exceptional catalysts, especially for the polymerization of styrene. Molecular weights were generally in good agreement with theoretical values, with PDIs as low as 1.11. Polymerization of styrene utilizing chloro-substituted amine-bis(phenolate) iron(III) halides proceeds rapidly, affording excellent control over both molecular weights and PDIs. Kinetic studies illustrated the controlled nature of the polymerization and polymer end-group analysis suggests that control is imparted by cooperation between ATRP and OMRP mechanisms.

The present iron complexes are also effective catalysts for MMA polymerization, although significant differences in the mechanism of control are implied. Polymerization with MMA monomer is almost certainly not operating via DT-OMRP. Although product polymer molecular weights are in fairly good agreement to those calculated using the monomer to catalyst ratio, PDIs of MMA polymer were broad in all cases indicating a lack of control. Best results for MMA polymerization were obtained when reactions were carried out in solution (1:1 w/w) with excess radical initiator (3 or 6 eq.). The difference in mechanism between styrene and MMA may be due to the stronger iron-carbon bond formed during MMA polymerization.

### Example 8

### Tridentate amine-bis(phenolate) Ligands and Corresponding iron(III) amine-bisphenolate Complexes

This example describes the synthesis and structure of six iron(III) complexes supported by tridentate amine-bis(phenolate) ligands, as shown below.

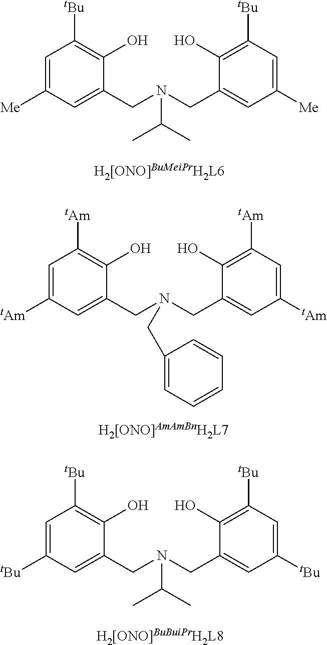

General Methods and Materials

H₂L6, H₂L7 and H₂L8 were synthesized in the presence of air. Unless otherwise stated, all iron complexes were synthesized under an atmosphere of dry oxygen-free nitrogen by means of standard Schlenk techniques or by using an MBraun LabmasterDP glove box. THE was stored over sieves and distilled from sodium benzophenone ketyl under nitrogen. Anhydrous toluene was purified using an MBraun solvent purification system. Anhydrous FeCl₃ (97%) was used for the synthesis of 10-20. Anhydrous FeBr₃ (99%) was obtained from Strem Chemicals for the preparation of 30-60. Reagents were purchased either from Strem, Aldrich or Alfa Aesar and used without further purification.

NMR spectra were recorded in CDCl₃ with a Bruker Avance III 300 MHz instrument with a 5 mm-multinuclear broadband observe (BBFO) probe. MALDI-TOF MS spectra were performed using an ABI QSTAR XL Applied Biosystems/MDS hybrid quadrupole TOF MS/MS system equipped with an oMALDI-2 ion source. Samples were prepared at a concentration of 10.0 mg/mL in toluene. Anthracene was used as the matrix, which was mixed at a concentration of 10.0 mg/mL. UV-vis spectra were recorded with an Ocean Optics USB4000+ fiber optic spectrophotometer. HR-MS spectra were recorded using a High Resolution MSD Waters Micromass GCT Premier spectrometer equipped with an electron impact ion source and a time-of-flight (oa-TOF) mass analyzer. Melting point data were collected on a MPA100 OptiMelt Automated Melting Point System. Elemental analyses were carried out by Canadian Micro-analytical Services Ltd. Delta, BC, Canada, or by Guelph Chemical Laboratories Ltd. Guelph, Ontario, Canada. The crystal structures were solved on a AFC8-Saturn 70 single crystal X-ray diffractometer from Rigaku/MSC, equipped with an X-stream 2000 low temperature system.

H₂[O₂N]^{BuMeiPr} (H₂L6):

To a stirred mixture of 2-t-butyl-4-methylphenol (20.398 g, 0.1232 mol) in 100 mL of deionized water was added 37% aqueous formaldehyde (10 mL, 0.1232 mol) followed by slow addition of isopropylamine (3.55 g, 0.0615 mol). The reaction was heated to reflux for 12 hours. Upon cooling, the reaction mixture separated into two phases. The upper phase was decanted and the remaining oily residue was triturated with cold methanol to give an analytically pure, white powder (16.25 g, 64%). ¹H NMR (300 MHz, CDCl₃, δ): 7.00 (s, ArH, 2H); 6.73 (s, ArH, 2H); 3.65 (s, CH₂, 4H); 3.16 (septet, ³J=5 Hz, CH, 1H); 2.24 (s, CH₃, 6H); 1.39 (s, CH₃, 18H); 1.17 (d, ³J=5 Hz, CH₃, 6H). ¹³C{¹H}NMR (75 MHz, 298 K, CDCl₃): δ 152.68 (Ar); 136.80 (Ar); 128.93 (Ar); 128.03 (Ar); 127.20 (Ar); 122.36 (Ar); 51.64 (CH₂); 48.33 (CH); 34.59 (C(CH₃)₃); 29.64 (C(CH₃)₃); 20.80 (ArCH₃); 16.64 (CH(CH₃)₂). HRMS (TOF MS EI+): (m/z): (M)⁺ calcd. For L1, 411.3137. found, 411.3143. MP range (° C.): 130.2-131.7.

H₂[O₂N]^{AmAmBn} (H₂L7):

To a stirred mixture of 2,4-di-t-amylphenol (28.829 g, 0.1232 mol) in 100 mL of deionized water was added 37% aqueous formaldehyde (10 mL, 0.1232 mol) followed by slow addition of benzylamine (6.59 g, 0.0615 mol). The reaction was heated to reflux for 12 hours. Upon cooling, the reaction mixture separated into two phases. The upper phase was decanted and the remaining white mass of solid material was triturated with cold methanol to give an analytically pure, white powder (27.91 g, 76%). ¹H NMR (300 MHz, CDCl₃, δ): 7.37 (s, ArH, 1H); 7.35 (s, ArH, 1H); 7.32 (s, ArH, 1H); 7.30 (s, ArH, 1H); 7.26 (s, ArH, 1H); 7.08 (d, J=1.6 Hz, ArH, 2H); 6.86 (d, J=1.6 Hz, ArH, 2H); 3.73 (s, NCH₂, 2H); 3.62 (s, ArCH₂, 4H); 1.87 (q, CH₂, 4H); 1.55 (q, CH₂, 4H); 1.34 (s, CH₃, 12H); 1.22 (s, CH₃, 12H); 0.64 (t, CH₃, 12H). ¹³C{¹H}NMR (75 MHz, 298 K, CDCl₃): δ 151.98 (Ar); 139.51 (Ar); 137.62 (Ar); 134.09 (Ar); 129.59 (Ar); 128.93 (Ar); 127.85 (Ar); 125.86 (Ar); 125.80 (Ar); 121.15 (Ar); 58.51 (CH₂); 56.95 (CH₂); 38.49 ((CH)₂C(CH₂CH₃)); 37.27 ((CH₃)₂C(CH₂CH₃)); 37.21 ((CH₃)₂C(CH₂CH₃)); 33.00 ((CH₃)₂C(CH₂CH₃)); 28.60 ((CH₃)₂C(CH₂CH₃)); 27.75 ((CH₃)₂C(CH₂CH₃)); 9.58 ((CH₃)₂C(CH₂CH₃)); 9.20 ((CH₃)₂C(CH₂CH₃)). HRMS (TOF MS EI+): (m/z): [M]⁺ calcd. For L2, 599.4702. found, 599.4711. MP range (° C.): 127.4-128.9.

H₂[O₂N]^{BuBuiPr} (H₂L8):

To a stirred mixture of 2,4-di-t-butylphenol (26.491 g, 0.1232 mol) in 100 mL of deionized water was added 37% aqueous formaldehyde (10 mL, 0.1232 mol) followed by slow addition of isopropylamine (3.55 g, 0.0615 mol). The reaction was heated to reflux for 12 hours. Upon cooling, the reaction mixture separated into two phases. The upper phase was decanted and the remaining light orange solid was triturated with cold methanol to give an analytically pure, white powder (17.32 g, 57%). ¹H NMR (300 MHz, CDCl₃, δ): 7.21 (s, ArH, 2H); 6.92 (s, ArH, 2H); 3.71 (s, CH₂, 4H); 3.17 (sp, ³J=5 Hz, CH, 1H); 1.39 (s, CH₃, 18H); 1.28 (s, CH₃, 18H); 1.18 (d, ³J=5 Hz, CH₃, 6H). ¹³C{¹H}NMR (300 MHz, 298 K, CDCl₃): δ 152.60 (ArCOH); 141.43 (Ar); 136.02 (Ar); 125.03 (Ar); 123.41 (Ar); 121.63 (Ar); 52.00 (NCH(CH₃)₂); 48.40 (ArCH₂); 34.88 (C(CH₃)₃); 34.18 (C(CH₃)₃); 31.67 (C(CH₃)₃); 29.70 (C(CH₃)₃); 16.66 (CH(CH₃)₂). HRMS (TOF MS EI+): (m/z): [M]⁺ calcd. For L3, 495.4076. found, 495.4063. MP range (° C.): 142.5-143.3. IR (neat): v=3196, 2958, 2905, 2865, 1606, 1476, 1451, 1391, 1362, 1290, 1225, 1207, 1157, 1123, 1078, 1027, 995, 967, 935, 879, 824, 792, 755, 722, 682, 653, 600, 540, 503 cm⁻¹.

[NEt₃H]⁺[FeCl₂L6]⁻ (10): To a THF solution (50 mL) of recrystallized L6 (2.00 g, 4.87 mmol) was added a solution of anhydrous FeCl₃ (0.800 g, 4.93 mmol) in THF resulting in an intense purple solution. To this solution was added triethylamine (1.00 g, 9.86 mmol) and the resulting mixture was stirred for 2 hours. After stirring, the dark purple solution was filtered through Celite. Removal of solvent under vacuum yielded a dark purple product. Crystals suitable for X-ray diffraction were obtained by slow evaporation of a toluene solution (1.693 g, 55%). Anal. Calcd for C₃₃H₅₅Cl₂FeN₂O₂ (plus 1.3 equivalents of co-crystallized toluene); C, 66.68; H, 8.69; N, 3.69. Found: C, 66.73; H, 8.92; N, 3.44.

FeCl(THF)L7 (20):

To a TI-F solution (50 mL) of recrystallized L7 (2.00 g, 3.33 mmol) was added a solution of anhydrous FeCl₃ (0.597 g, 3.33 mmol) in THF resulting in an intense purple solution. To this solution was added triethylamine (0.674 g, 6.66 mmol) and the resulting mixture was stirred for 2 hours. After stirring, the dark purple solution was filtered through Celite. Removal of solvent under vacuum yielded a dark purple product (1.809 g, 71%). The purple product was dissolved in minimal toluene and was placed in the freezer for 48 hours were a thin layer of white precipitate appeared at the bottom of the reaction flask. The mother liquor was decanted and passed through Celite. Crystals suitable for X-ray diffraction were obtained by slow evaporation of the toluene solution (1.408 g, 56%). Anal. Calcd for C₄₅H₆₇ClFeNO₃: C, 70.99; H, 8.87; N, 1.84. Found: C, 71.25; H, 9.03; N, 2.10. (MALDI-TOF) m/z (%, ion): 599.445 (100, [M-Fe—Cl-THF]⁺), 653.375 (40, [M-Cl-THF}]⁺), 688.328 (8, [M-THF}]⁺). UV-vis (methanol) λₘₐₓ, nm (ε): 600 (2750), 330 (3950), 250 (6610).

FeBr(THF)L7 (30):

A THF solution (50 mL) of recrystallized L2 (2.00 g, 3.33 mmol) was added dropwise to a NaH suspension (0.320 g, 13.33 mmol) in THF at −78° C. Upon return to room temperature, the sodium salt of the ligand was added dropwise to a THF solution of anhydrous FeBr₃ (0.985 g, 3.33 mmol) at −78° C. resulting in an intense purple solution. After stirring for 2 hours, the solvent was removed via vacuum to give a dark purple powder. The dark purple product was then extracted with minimal toluene and the resulting dark purple solution was filtered through Celite. Crystals suitable for X-ray diffraction were obtained by slow evaporation of the toluene solution (2.255 g, 84%). Anal. Calcd for C₄₅H₆₇BrFeNO₃: C, 67.08; H, 8.38; N, 1.74. Found: C, 66.87; H, 8.12; N, 2.05. (MALDI-TOF) m/z (%, ion): 599.445 (40, [M-Fe—Br-THF]⁺), 653.363 (100, [M-Br-THF}]⁺), 734.288 (5, [M-THF]⁺), 805.225 (1, [M]⁺).

FeBr₂L6H (40):

A THF solution (50 mL) of recrystallized L6 (2.00 g, 4.86 mmol) was added dropwise to a NaH suspension (0.467 g, 19.45 mmol) in THF at −78° C. Upon progressive return to room temperature, the sodium salt of the ligand was added dropwise to a THF solution of anhydrous FeBr₃ (1.44 g, 4.86 mmol) at −78° C. resulting in an intense purple solution. After stirring for 2 hours, the solvent was removed via vacuum to give a dark purple powder. The dark purple product was then washed with minimal toluene and the resulting dark purple solution was filtered through Celite. Crystals suitable for X-ray diffraction were obtained by slow evaporation of the toluene solution (1.958 g, 64%). Anal. Calcd for C₂₇H₄₀Br₂FeNO₂: C, 51.78; H, 6.44; N, 2.24. Found: C, 51.53; H, 6.18; N, 2.07. (MALDI-TOF) m/z (%, ion): 412.296 (100, [M-Fe-2Br—H]⁺), 465.215 (7, [M-2Br—H]⁺), 545.135 (3, [M-Br—H]⁺).

[FeL8(μ-OH)]₂ (50):

A 1.6 M hexane solution of n-butyllithium (5.50 mL, 8.87 mmol) was added via syringe to a stirred solution of L8 (2.00 g, 4.03 mmol) in THF (50 mL) at −78° C. Upon return to room temperature, the lithiated ligand (clear pale yellow solution) was transferred via cannula to a solution of anhydrous FeBr₃ (1.19 g, 4.03 mmol) in THF (30 mL) at −78° C. After stirring for 2 hours, the solvent was removed via vacuum to give a dark purple powder. The dark purple product was then extracted with minimal toluene and the resulting dark purple solution was filtered through Celite. Dark brown crystals suitable for X-ray diffraction were obtained by slow evaporation of the toluene solution (3.813 g, 83%). Anal. Calcd for C₆₆H₁₀₄Fe₂N₂O₆: C, 69.95; H, 9.25; N, 2.47. Found: C, 70.12; H, 8.98; N, 2.65. (MALDI-TOF) m/z (%, ion): 496.479 (100, [M-FeOH]⁺), 549.399 (10, [M-OH]⁺), 564.394 (7, [M]).

FeBr₂L8H (60):

A 1.6 M hexane solution of n-butyllithium (5.50 mL, 8.87 mmol) was added via syringe to a stirred solution of L8 (2.00 g, 4.03 mmol) in THF (50 mL) at −78° C. Upon return to room temperature, the lithiated ligand (clear pale yellow solution) was transferred via cannula to a solution of anhydrous FeBr₃ (1.19 g, 4.03 mmol) in THF (30 mL) at −78° C. After stirring for 2 hours, the solvent was removed via vacuum to give a dark purple powder. The dark purple product was then extracted with minimal toluene and the resulting dark purple solution was filtered through Celite. Dark purple crystals suitable for X-ray diffraction were obtained by slow evaporation of the toluene solution (2.156 g, 76%). Anal. Calcd for C₃₃H₅₂Br₂FeNO₂: C, 55.79; H, 7.38; N, 1.97. Found: C, 55.61; H, 7.19; N, 2.11. (MALDI-TOF) m/z (%, ion): 710.468 (2, [M]⁺), 549.260 (10, [M-2Br—H]⁺), 492.320 (100, [M-Fe-2Br—H]⁺).

Results

The tridentate amine-bis(phenol) ligand precursors, abbreviated H₂[O₂N]^{RR′R″} (where R=^{t}Bu, R=Me, R=isopropyl (H₂L6); R=^{t}Am, R=^{t}Am, R=benzyl (H₂L7) and R=^{t}Bu, R=^{t}Bu, R=isopropyl (H₂L8)) were readily synthesized by a modified Mannich condensation reaction, in which the desired 2,4-disubstituted phenol, amine and formaldehyde were heated to reflux in water for 12 hours (Scheme 7) (F. M. Kerton, S. Holloway, A. Power, R. G. Soper, K. Sheridan, J. M. Lynam, A. C. Whitwood, C. E. Willans, Can. J. Chem., 2008, 86, 435-443). This class of ligands can be complexed to transition metals by several routes. For example, metallation with alkali metal reagents can be accomplished using ⁿBuLi or NaH to generate M₂[L] salts where M=Li or Na, respectively. Several structurally characterized examples of dilithiated amine-bis(phenolate) compounds have recently been reported (F. M. Kerton, C. Koak, K. Luttgen, C. E. Willans, R. J. Webster, A. C. Whitwood, Inorg. Chem. Acta. Can. J. Chem., 2006, 359, 2819; C. A. Huang, C. T. Chen, Dalton Trans., 2007, 5561, and R. Dean, S. Granville, L. Dawe, A. Decken, K. Hattenhauer, C. Kozak, Dalton Trans., 2010, 39, 548.).

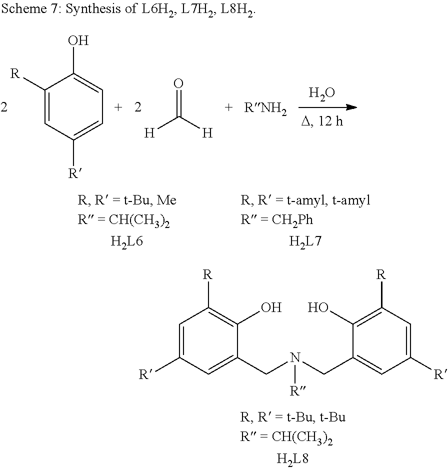

According to previous work in the Kozak group, when FeCl₃ reacts with a tridentate amine-bis(phenolate) ligand in the presence of a NEt₃, the resulting complexes exist as halide-bridged dimers in the solid state giving distorted trigonal bipyramidal iron(III) ions (X. Qian, L. Dawe, C. Kozak, Dalton Trans., 2010, 39, 1). However, from recent results, it was found that other Fe(III) complexes can be generated depending on the purification procedures employed and the steric requirements of the amine-bis(phenolate) backbone. The ligands H₂L6 and H₂L7 react with FeCl₃ in THF to generate the monometallic complexes 10 and 20, respectively (Scheme 8). The resulting dark purple solutions were neutralized using NEt₃. From these solutions, complexes 10 and 20 were isolated in moderate to high yield.

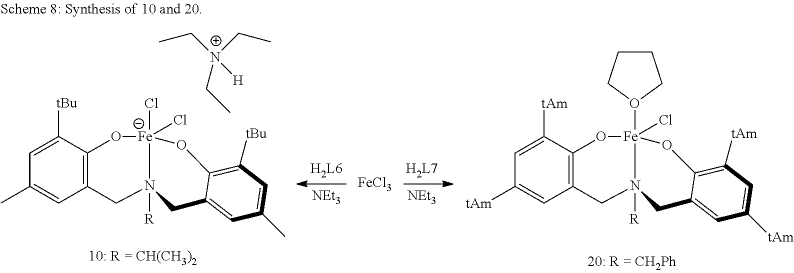

Since the ligand backbone in 20 contains very bulky t-amyl substituents, one reason why the iron(III) THF adduct was formed in favor of the chloride-bridged dimer may be that the dimer formation is sterically unfavored. MALDI-TOF mass spectrometry was carried out on 20 using anthracene as the matrix. The mass spectrum of 20 showed characteristic fragment ions. The THF ligand is easily lost from the parent ion in 20 ([M-THF]⁺). An intense [M-THF-Cl]⁺ peak was also observed in the mass spectrum of 20.

The sodium salt prepared from the reaction between Nail and H₂L7 in THY at −78° C. was reacted with anhydrous FeBr₃ in THF at −78° C., to generate an immediate color change to dark purple (Scheme 9). From this solution, complex 30 was isolated in high yield. Once again, since the ligand backbone in 30 contains very bulky t-amyl substituents, the monomer species is likely to hindered to dimerize (but there is enough space for THF to coordinate). The mass spectrum of 30 showed a very weak molecular ion peak, [M]⁺, and characteristic fragment ions. The THF ligand is easily lost from the parent ion in 30 ([M-THF]⁺). An intense [M-THF-Br]⁺ peak was also observed in the mass spectrum of 30. Surprisingly, when the sodium salt prepared from the reaction between Nail and H₂L6 in THF at −78° C. was reacted with anhydrous FeBr₃ in THF at −78° C., a zwitterionic tetrahedral iron(III) complex bearing two bromide ligands and a quaternized ammonium fragment was generated (40) instead of the suspected bromide-bridged dimer. A similar complex was previously reported in the Kozak group when NEt₃ was used as the base (X. Qian, L. Dawe, C. Kozak, Dalton Trans., 2010, 39, 1). In the mass spectrum of 40, there is a peak that represents the loss of one bromide ligand from the parent ion along with the proton of the central nitrogen atom ([M-Br—H]⁺). There is also a peak that represents the loss of both bromide ligands and the proton of the central N atom ([M-2Br—H]⁺) from the parent ion.

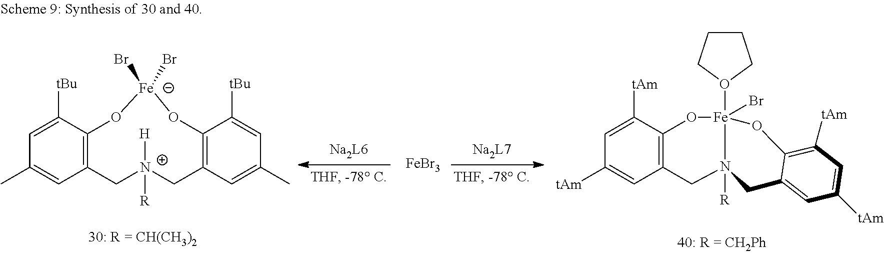

The lithium salt prepared from the reaction between ⁿBuLi and H₂L8 in THF at −78° C. was reacted with anhydrous FeBr₃ in THF at −78° C., to generate an immediate color change to dark purple (Scheme 10). From this solution, complex 50 was isolated in high yield instead of the suspected bromide-bridged dimer. According to the work reported by Attia and co-workers, treatment of a monomeric Fe(III) species (with coordinated monoanionic ligands) with a strong base (such as KOH) at room temperature leads to a μ-dihydroxo bridging structure core (S. Attia, M. F. El-Shahat, Polyhedron, 2007, 26, 791). If water contamination occurred during the synthesis of 50, LiOH could have been generated due to hydrolysis of the lithiated amine-bis(phenolate) ligand and may have reacted with the desired product in solution to generate the observed hydroxyl-bridged dimer. In the mass spectrum of 50, a molecular ion peak ([M]⁺) is evident. An intense [M-OH]⁺ peak was also observed in the mass spectrum of 50. In an attempt to successfully isolate [FeL3(μ-Br)]₂ (instead of ([FeL3(μ-OH)]₂ (50)) the reaction between the lithiated ligand of H₂L3 and FeBr₃ was repeated as shown in Scheme 10. Surprisingly, the zwitterionic tetrahedral iron(III) complex 60, bearing two bromide ligands and a quaternized ammonium fragment was isolated in high yield from the resulting dark purple solution. In the mass spectrum of 60, there exists a very weak molecular ion peak ([M]⁺). There also exists a peak in the mass spectrum of 60 which represents the loss of both bromide ligands and the proton of the central nitrogen atom ([M-2Br—H]⁺) from the parent ion.

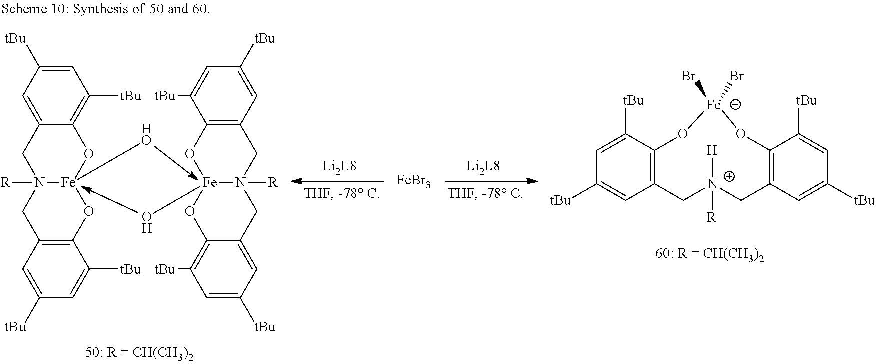

Structural Characterization

Molecular Structure of 10:

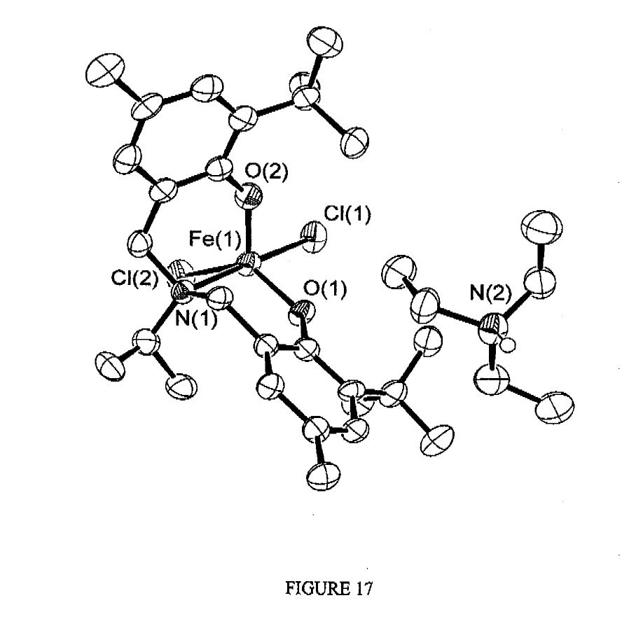

Single crystals of 10 suitable for X-ray diffraction were obtained from a saturated toluene solution at −35° C. inside a nitrogen filled glove box. The solid state molecular structure of 10 is shown in FIG. 17, while crystallographic data and selected metric parameters are shown in Table 21 and Table 22, respectively. In the solid state, 10 exhibits a monomeric structure having a trigonal bipyramidal iron(III) centre with a formal negative charge (“ate” complex). In the solid state molecular structure of 10, there exists a toluene molecule sandwiched between repeating units of the anion (“ate” complex) and cation (triethylammonium). Since there are no distinguishable π-π interactions within the structure, it is likely that the toluene molecule is caged within the structure as the result of ionic (Coulombic) intermolecular forces between the anion and cation units.

Elemental analysis performed on a recrystallized sample of 10 supports this reasoning. The equatorial plane of the Fe^{III} on in 10 consists of two phenolate oxygens, O(1) and O(2), and a chloride ion, Cl(2), where the sum of bond angles is 359.69° indicating near perfect planarity. The iron atom is displaced 0.06 Å above the equatorial plane. The amine nitrogen donor (N(1)) and the chloride ion Cl(1) occupy the apical sites, giving a Cl(1)-Fe(1)-N(1) bond angle of 178.85(7)° which is close to the ideal linear geometry. The cis-orientated chloride ligands are nearly orthogonal with a Cl(1)-Fe(1)-Cl(2) bond angle of 91.42(5)°. The distorted trigonal bipyramidal coordination environment of the Fe^{III} ion possesses a trigonality index parameter, τ, value of 0.837 [as defined by Addison and Reedijk, τ=(β−α)/60, where β represents the largest angle about the metal centre and α represents the second largest angle about the metal centre. For perfect trigonal bipyramidal and square pyramidal geometries the τ values are one and zero, respectively] (A. W. Addison, T. N. Rao, J. Reedijk, J. van Rijn, G. C. Verschoor, J. Chem. Soc., Dalton Trans. 1984, 1349).

TABLE 21
Crystallographic and Structure Refinement Data for 10, 20, 30, 40 and 60 and H₂L8.

| Compound | | | | | | |
|---|---|---|---|---|---|---|
| reference | 10 | 20 | 30 | 40 | 60 | H₂L8 |
| Chemical | C₄₀H₆₃Cl₂FeN₂O₂ | C₄₅H₆₇ClFeNO₃ | C₄₅H₆₇BrFeNO₃ | C_{44.50}H₆₀Br₂FeNO₂ | C₄₀H₆₀Br₂FeNO₂ | C₃₃H₅₃NO₂ |
| formula | | | | | | |
| Colour | Dark Red | Red | Dark Red | Black | Dark Red | Colorless |
| Habit | Prism | Prism | Prism | Prism | Prism | Prism |
| Formula | 730.70 | 761.33 | 805.78 | 856.62 | 802.57 | 496.76 |
| Mass | | | | | | |
| Crystal | Triclinic | Monoclinic | Monoclinic | Monoclinic | Triclinic | Mononclinic |
| system | | | | | | |
| a [Å] | 11.162(3) | 25.441(12) | 39.512(3) | 14.816(4) | 10.5127(10) | 15.185(4) |
| b [Å] | 11.397(4) | 10.907(4) | 10.9112(4) | 16.729(4) | 13.6960(14) | 11.707(3) |
| c [Å] | 17.686(6) | 31.379(15) | 25.084(2) | 18.202(5) | 15.3413(15) | 18.596(4) |
| α [°] | 83.003(14) | 90 | 90 | 90 | 68.421(5) | 90 |
| β [°] | 75.944(13) | 94.00(3) | 126.271(3) | 107.386(3) | 79.693(6) | 108.298(4) |
| γ [°] | 69.005(11) | 90 | 90 | 90 | 77.951(6) | 90 |
| Unit cell V | 2036.1(11) | 8686(7) | 8718.8(11) | 4305.4(20) | 1996.1(3) | 3138.7(12) |
| [Å³] | | | | | | |
| Temperature | 163(1) | 163(1) | 163(1) | 163(1) | 163(1) | 158(1) |
| [K] | | | | | | |
| Space group | P-1 (#2) | I2/a (#15) | C2/c (#15) | P2₁/c (#14) | P-1 (#2) | P2₁/c (#14) |
| Z | 2 | 8 | 8 | 4 | 2 | 4 |
| D_{c}/g cm⁻³ | 1.192 | 1.164 | 1.228 | 1.321 | 1.335 | 1.049 |
| Radiation | MoKα | MoKα | MoKα | MoKα | MoKα | MoKα |
| type | | | | | | |
| Absorption, | 0.535 | 0.446 | 1.302 | 2.246 | 2.412 | 0.630 |
| μ [mm⁻¹] | | | | | | |
| F(000) | 786 | 3288 | 3432 | 1784 | 838 | 1096 |
| Reflections | 17721 | 16461 | 54433 | 54221 | 14400 | 40638 |
| measured | | | | | | |
| Independant | 8338 | 7386 | 9027 | 8910 | 6952 | 6483 |
| refl's | | | | | | |
| Rᵢₙₜ | 0.0574 | 0.1000 | 0.0364 | 0.0588 | 0.0962 | 0.0369 |
| R₁ (I > | 0.0634 | 0.1181 | 0.0622 | 0.0554 | 0.0991 | 0.0830 |
| 2σ(I))^{[a]} | | | | | | |
| wR(F²) (I > | 0.1905 | 0.3701 | 0.1715 | 0.1413 | 0.2578 | 0.2384 |
| 2σ(I))^{[b]} | | | | | | |
| Goodness of | 1.093 | 1.094 | 1.093 | 1.104 | 1.035 | 1.157 |
| fit on F² | | | | | | |

[a]R₁ = Σ(|Fₒ| − |F_{c}|)/Σ|Fₒ|).
[b]wR₂ = [Σ(w(Fₒ² − F_{c}²)²)/Σw(Fₒ²)²]^{1/2}

TABLE 22
Selected bond lengths (Å) and bond angles (°) of 10,
[Fe[ONO]^{BuMenPr}(μ-Cl)]₂ (Dimer A) and
(Fe[ONO]^{BuMenPr}(μ-Cl)]₂ (Dimer B). Symmetry operators
used to generate equivalent atoms: (*) −x + 1, −y + 1, −z + 1.

| | Dimer A | Dimer B | 10 |
|---|---|---|---|
| Fe(1)-O(1) | 1.818(3) | 1.8276(13) | 1.855(2) |
| Fe(1)-O(2) | 1.817(3) | 1.8222(12) | 1.848(3) |
| Fe(1)-N(1) | 2.183(4) | 2.1819(10) | 2.255(3) |
| Fe(1)-Cl(1) | 2.298(2) | 2.3290(4) | 2.3618(4) |
| Fe(1)-Cl(1)* | 2.4911(18) | | |
| Fe(1)-Cl(2) | | 2.5025(3) | 2.3038(14) |
| O(1)-Fe(1)-O(2) | 124.63(14) | 119.36(5) | 114.58(12) |
| N(1)-Fe(1)-Cl(1) | 93.92(10) | 93.59(3) | 178.85(7) |
| N(1)-Fe(1)-Cl(1)* | 178.32(9) | | |
| N(1)-Fe(1)-Cl(2) | | 177.28(3) | 89.35(9) |
| Cl(1)-Fe(1)-Cl(1)* | 87.36(6) | | |
| Cl(1)-Fe(1)-Cl(2) | | 84.341(14) | 91.42(5) |
| Fe(1)-Cl(1)-Fe(1)* | 92.64(6) | | |
| Fe(1)-Cl(1)-Fe(2) | | 95.384(14) | |
| O(1)-Fe(1)-Cl(1) | 113.18(11) | 114.96(4) | 91.49(8) |
| O(1)-Fe(1)-Cl(1)* | 89.86(11) | | |
| O(1)-Fe(1)-Cl(2) | | 88.91(3) | 128.60(10) |
| O(2)-Fe(1)-Cl(1) | 122.08(12) | 125.52(4) | 92.71(9) |
| O(2)-Fe(1)-Cl(1)* | 89.41(11) | | |
| O(2)-Fe(1)-Cl(2) | | 92.60(3) | 116.51(8) |
| O(1)-Fe(1)-N(1) | 88.99(13) | 90.38(4) | 87.35(10) |
| O(2)-Fe(1)-N(1) | 90.62(13) | 90.03(4) | 87.71(11) |

Mononuclear trigonal bipyramzidal iron(III) complexes of related tetradentate diamine-bis(phenolate) ligands (abbreviated [O₂NN′], where N′ represents a pendant dimethylaminoethyl or pyridyl arm) have been previously reported (K. Hasan, C. Fowler, P. Kwong, A. K. Crane, J. L. Collins, C. M. Kozak, Dalton Trans., 2008, 2991). The Fe—Cl bond lengths in FeCl[O₂NN′]^{BuMePy} and FeCl[O₂NN′]^{BuMeNMe2} were found to be 2.3051(10) and 2.2894(5) Å respectively, which are very similar to the Fe—Cl(2) interaction observed in the equatorial plane of 10. The Fe—Cl(1) bond length (2.3618(13) Å) in 10, were Cl(1) is trans to a hard nitrogen donor, is slightly longer than the Fe—Cl bond length observed in FeCl[O₂NN′]^{BuMePy} and also slightly longer than the Fe—Cl bond length observed in FeCl[O₂NN′]^{BuMeNMe2} (were Cl is also trans to a hard nitrogen donor). 10 has a Fe—N(1) distance of 2.255(3) Å which is very similar to the Fe—N bond lengths reported in FeCl[O₂NN′]^{BuMePy} 2.2706(15) Å) and FeCl[O₂NN′]^{BuMeNMe2} (2.248(2) Å). The phenolate oxygen atoms in 10 exhibit bond distances of 1.855(2) and 1.848(2) Å for Fe(1)-O(1) and Fe(1)-O(2), respectively. These interactions are only slightly shorter than those observed in Kozak's FeCl[O₂NN′] complexes, where average Fe—O distances of 1.86 Å are observed.

The coordination geometry around iron(III) in 10 is very closely related to a series of iron(III) chloride-bridged dimers ([Fe[ONO]^{BuMenPr}(μ-Cl)]₂ (Dimer A) and [Fe[ONO]^{BuMeBn}(μ-Cl)]² (Dimer B)) previously reported (X. Qian, L. Dawe, C. Kozak, Dalton Trans., 2010, 39, 1). Selected metric parameters for (Dimer A) and (Dimer B) can be found in Table 22. Like 10, the five-coordinate trigonal bipyramidal iron(III) centre(s) in Dimer A and Dimer B are composed of two chloride ions along with two phenolate oxygen donor atoms and a central amine nitrogen atom originating from a tridentate amine-bis(phenolate) backbone. The axial Fe—Cl bond length in 10 (2.3618(4) Å) is slightly shorter than the axial Fe—Cl bond lengths found in Dimer A (2.4911(8) Å) and Dimer B (2.5025(3) Å). The equatorial Fe—Cl(2) bond length in 10 (2.3038(14) Å) is intermediate to the equatorial Fe—Cl bond lengths reported in Dimer A (2.298(2) Å) and Dimer B (2.3290(4) Å). The Fe—N(1) distance of 2.255(3) Å observed in 10 is longer than the observed Fe—N distances found in both chloride-bridged dimers. The Fe—O distances in 10 are 1.855(2) and 1.848(3) Å for Fe(1)-O(1) and Fe(1)-O(2), respectively, which are longer than the distances reported between iron and the phenolate oxygen atoms in Dimer A and Dimer B. Since the iron centre in 10 has a formal negative charge, the anionic oxygen donors may be slightly repelled by the metal centre. From an electronic perspective, this may account for the longer Fe—O distances observed in 10. Of course, in the case of both Dimer A and Dimer B, steric hindrance originating from the presence of two large amine-bis(phenolate) ligands about the two iron(III) centres may also be a major contributor. Two phenolate oxygen donor atoms and a bridging chloride occupy the equatorial plane around each iron ion, where the sum of bond angles is 359.89° in Dimer A and 359.84° in Dimer B. In comparison to both chloride-bridged dimers, the sum of bond angles about the equatorial plane in 10 (359.69°) is slightly lower. The amine nitrogen donor and a bridging chloride ion take up the axial positions, giving a Cl(1)*-Fe(1)-N(1) bond angle of 178.32(9)° in Dimer A and Cl(2)-Fe(1)-N(1) bond angle of 177.28(3)° in Dimer B. Complex 10 has a Cl(1)-Fe(1)-N(1) bond angle of 178.85(7)° which is closer to the ideal linear geometry. The cis-orientated chloride ligands are nearly orthogonal with a Cl—Fe—Cl bond angle of 87.36(4)° in Dimer A and 84.341(14)° in Dimer B. The Cl—Fe—Cl bond angle in 10 is 91.42(5)°, which is closer to the perfect orthogonal angle of 90°.

In 2002, Leznoff and co-workers reported a five-coordinate iron(III) chloride-bridged dimer with a distorted trigonal bipyramidal geometry (G. Mund, R. J. Batchelor, R. D. Sharma, C. Jones, D. B. Leznoff, J. Chem. Soc., Dalton Trans. 2002, 136). Unlike the coordination environment of 10, which contains two anionic oxygen donor atoms and a central nitrogen donor, the iron(III) centre in {FeCl[^{t}BuN(SiMe₂)]₂O}₂ is composed of two anionic nitrogen donor atoms and a central, neutral O-donor. The Fe—Cl bond lengths in {FeCl[^{t}BuN(SiMe₂)]₂O}₂ are 2.3181(19) and 2.4652(17) Å whereas the corresponding distances in 10 are 2.3618(4) and 2.3038(14) Å. The Cl—Fe—Cl bond angle in {FeCl[^{t}BuN(SiMe₂)]₂O}₂ is 86.75(6)°, which is lower than the Cl—Fe—Cl angle observed in 10 (91.42(5)°) and intermediate to those observed in Dimer A (87.36(6)° and Dimer B (84.341(14)°). The central, neutral O-donor in {FeCl[^{t}BuN(SiMe₂)]₂O}₂ is only weakly bonded to the iron centre, showing a Fe—O bond distance of 2.597(4) Å. However, the anionic nitrogen donors in {FeCl[^{t}BuN(SiMe₂)]₂O}₂ show Fe—N bond lengths of 1.894(4) and 1.887(5) Å which are slightly longer than the Fe—N distance of 2.255(3) Å found in 10. The sum of bond angles about the equatorial plane in {FeCl[BuN(SiMe₂)]₂O}₂ is only 332.23°, compared to nearly 3600 in 10, Dimer A and Dimer B. This suggests that the iron centre in {FeCl[^{t}BuN(SiMe₂)]₂O}₂ is more closely tetrahedral in geometry whereas the iron centres in 20, Dimer A and Dimer B possess five strong metal-ligand interactions.

Molecular Structure of 20 and 30:

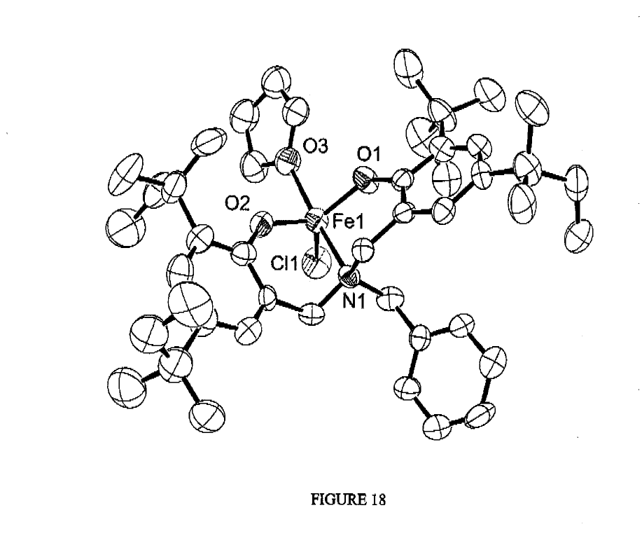

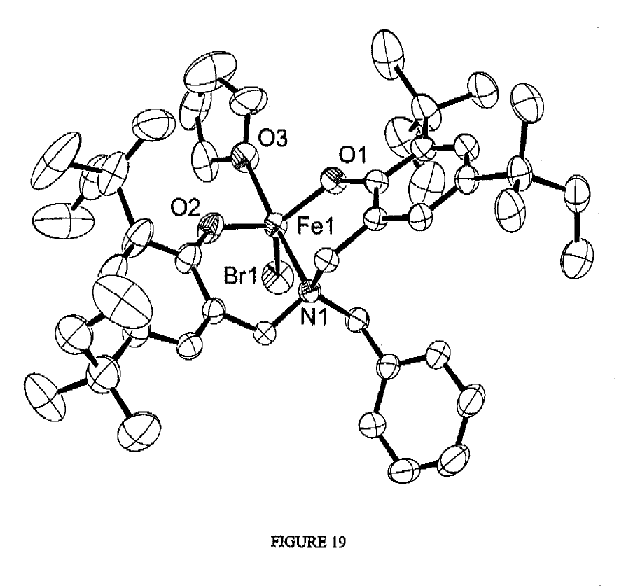

Slow evaporation of toluene solutions of 20 and 30 under a N₂ atmosphere in a glove box provided single crystals suitable for X-ray diffraction analysis. The solid state molecular structures of 20 and 30 are shown in FIG. 18 and FIG. 19, respectively. The crystallographic data and selected metric parameters of 20 and 30 are shown in Table 21 and Table 23, respectively.

TABLE 23
Selected bond lengths (Å) and bond angles
(°) of 20, 30 and FeCl(O₂NO']^{BuMeFurf}.

| | 20 | 30 | FeCl(O₂NO']^{BuMeFurf} |
|---|---|---|---|
| Fe(1)-O(1) | 1.854(6) | 1.8491(18) | 1.850(2) |
| Fe(1)-O(2) | 1.848(6) | 1.842(4) | 1.854(2) |
| Fe(1)-O(3) | 2.151(6) | 2.145(2) | 2.074(3) |
| Fe(1)-N(l) | 2.190(6) | 2.185(2) | 2.223(3) |
| Fe(1)-Cl(1) | 2.237(3) | | 2.2739(10) |
| Fe(1)-Br(1) | | 2.3808(8) | |
| O(1)-Fe(1)-O(2) | 123.7(3) | 124.80(14) | 118.39(10) |
| N(1)-Fe(1)-Cl(1) | 96.12(17) | | 165.69(8) |
| N(1)-Fe(1)-Br(1) | | 96.79(8) | |
| N(1)-Fe(1)-O(3) | 172.0(2) | 171.77(12) | 75.79(10) |
| Cl(1)-Fe(1)-O(3) | 91.72(18) | | 89.98(8) |
| Br(1)-Fe(1)-O(3) | | 91.36(10) | |
| O(1)-Fe(1)-Cl(1) | 121.49(20) | | 100.81(8) |
| O(1)-Fe(1)-Br(1) | | 120.17(11) | |
| O(1)-Fe(1)-O(3) | 86.7(2) | 86.89(9) | 119.00(11) |
| O(2)-Fe(1)-Cl(1) | 114.7(2) | | 96.60(8) |
| O(2)-Fe(1)-Br(1) | | 114.88(9) | |
| O(2)-Fe(1)-O(3) | 88.6(2) | 88.17(12) | 119.60(11) |
| O(1)-Fe(1)-N(1) | 88.0(2) | 88.03(8) | 87.62(10) |
| O(2)-Fe(1)-N(1) | 89.4(2) | 89.41(11) | 89.37(10) |

For 20 and 30, the coordination around the iron atom is distorted trigonal bipyramidal with the trigonality index value (r) of 0.805 in 20 and 0.783 in 30. The metal is bonded to two phenolate oxygen atoms and a halide ion (a chloride ion in 20 and a bromide ion in 3), which define the trigonal plane of the bipyramid. In 20 and 30, the sum of bond angles about the equatorial plane is 359.89° and 359.85° respectively, indicating near perfect planarity. The central nitrogen atom of the ligand and the oxygen atom of the THF ligand occupy the apical sites of 20 and 30, giving a O(3)-Fe(1)-N(1) bond angle of 172.0(2)° and 171.77(12)°, respectively. The O(3)-Fe(1)-N(1) angle (for both 20 and 30) is considerably distorted from the ideal linear geometry; it is bent away from the phenolate groups and directed toward the halide ion. The Fe—O distances in 20 are 1.854(6) and 1.848(6) Å for Fe(1)-O(1) and Fe(1)-O(2), respectively. The iron(III) bromide complex 30 displays shorter Fe—O bond lengths of 1.8491(18) Å for Fe(1)-O(1) and 1.842(4) Å for Fe(1)-O(2) implying the presence of a slightly stronger iron-oxygen overlap. The Fe(1)-Cl(1) distance of 2.237(3) Å in 20 is shorter than the Fe—Cl distances found in 1, Dimer A and Dimer B. In addition, the Fe—Cl distance observed in 20 is slightly shorter than the Fe—Cl distances reported in similar iron(III) trigonal bipyramidal complexes possessing tetradentate amine-bis(phenolate) ligands. (K. Hasan, C. Fowler, P. Kwong, A. K. Crane, J. L. Collins, C. M. Kozak, Dalton Trans., 2008, 2991) The Fe(1)-Br(1) distance of 2.3808(8) Å in 30 is longer than the Fe(1)-Cl(1) distance of 2.237(3) Å in 20. However, the Fe(1)-Br(1) distance in 30 is shorter than Fe—Br distances reported in other five-coordinate iron(III)-bromide complexes (M. D. Fryzuk, D. B. Leznoff, E. S. F. Ma, S. J. Rettig, V. G. Young, Organometallics, 1998, 17, 2313; and G. A. Abakumov, V. K. Cherkasov, M. P. Bubnov, L. G. Abakumova, V. N. Ikorskii, G. V. Romanenko, A. I. Poddel'sky, Russ. Chem. Bull., 2006, 55, 44). The central nitrogen donor in the ligand backbone exhibits a Fe—N(1) bond length of 2.190(6) Å in 20 and 2.185(2) Å in 30. These Fe—N distances are slightly shorter than the Fe—N(1) bond length found in 10 (2.255(3) Å). For 20 and 30, the Fe(1)-O(3) bond lengths are 2.151(6) and 2.145(2) Å respectively, implying that the oxygen atom of the THF ligand in both complexes share approximately the same degree of overlap with the iron(III) centre.

The coordination environment of 20 shares striking similarities with an iron(III)-chloride complex previously reported by the Kozak group. Like 20, the coordination geometry around the iron atom in FeCl[O₂NO′]^{BuMeFurf} is trigonal bipyramidal (R. Chowdhury, A. Crane, C. Fowler, P. Kwong, C. Kozak, Chem. Commun. 2008, 94). However, unlike 20, which possesses a tridentate amine-bis(phenolate) ligand (with bulky tert-amyl substituents), the iron atom in FeCl[O₂NO′]^{BuMeFurf} is supported by a tetradentate amine-bis(phenolate) ligand containing a pendant tetrahydrofurfuryl group. A comparison of selected metric parameters can be found in Table 3. In FeCl[O₂NO′]^{BuMeFurf}, two phenolate oxygen atoms and the furfuryl oxygen atom define the bipyramid. As seen in FIG. 18, the THF ligand in 20 is located in the axial position. The chloride ion of 20 and FeCl[O₂NO′]^{BuMeFurf} are also located in different planes about the iron(III) centre; the chloride ion is located in the equatorial plane of 20 and in the axial plane of FeCl[O₂NO′]^{BuMeFurf}. In 20, a shorter Fe—Cl bond length (2.237(3) Å) is observed compared to the Fe—Cl distance in FeCl[O₂NO′]^{BuMeFurf} (2.2739(10) Å) since the chloride ion in the latter compound is trans to the amine nitrogen donor. The Fe—O(3) bond length in FeCl[O₂NO′]^{BuMeFurf} (which originates from the chelating tetrahydrofurfuryl pendant arm) is shorter than the Fe—O(3) distance observed in 20. For 20 and FeCl[O₂NO′]^{BuMeFurf}, the coordination around the iron atom is distorted trigonal bipyramidal with the trigonality index value (τ) of 0.805 in 20 and 0.768 in FeCl[O₂NO′]^{BuMeFurf}.

Molecular Structure of 40, 60 and H₂L8:

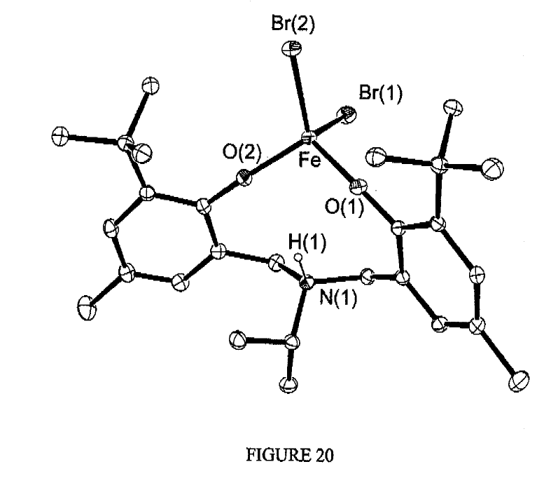

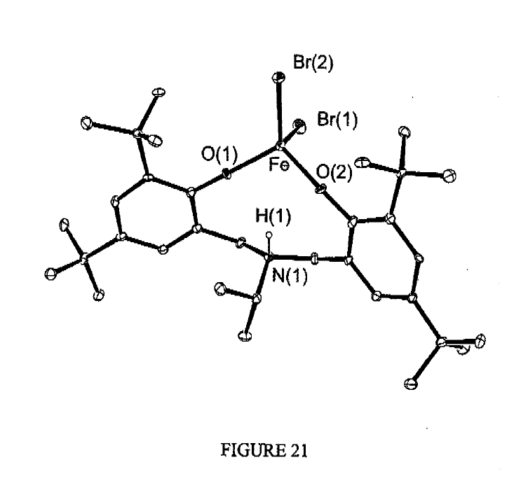

Single crystals of 40 and 60 suitable for X-ray diffraction were obtained from saturated toluene solutions at −35° C. inside a nitrogen filled glove box. The solid state molecular structures of 40 and 60 are shown in FIG. 20 and FIG. 21, respectively. The crystallographic data and selected metric parameters of 40 and 60 are shown in Table 21 and Table 24, respectively.

TABLE 24
Selected bond lengths (Å) and bond angles
(°) of 40, 60 and FeBr₂[O₂NH]^{BuMenPr}.

| | 40 | 60 | FeBr₂[O₂NH]^{BuMenPr} |
|---|---|---|---|
| Fe(1)-O(1) | 1.822(2) | 1.843(6) | 1.828(3) |
| Fe(1)-O(2) | 1.832(3) | 1.851(6) | 1.836(3) |
| Fe(1)-Br(1) | 2.3596(9) | 2.355(2) | 2.3569(7) |
| Fe(1)-Br(2) | 2.3491(8) | 2.3697(19) | 2.3723(7) |
| Fe•••N | 3.439(4) | 3.429(7) | 3.435(3) |
| O(1)-Fe(1)-O(2) | 106.38(13) | 105.9(3) | 105.24(15) |
| O(1)-Fe(1)-Br(1) | 108.43(8) | 108.9(2) | 110.72(9) |
| O(1)-Fe(1)-Br(2) | 110.90(9) | 110.8(2) | 109.24(15) |
| O(2)-Fe(1)-Br(1) | 110.71(9) | 107.5(3) | 112.87(10) |
| O(2)-Fe(1)-Br(2) | 110.08(9) | 112.0(2) | 108.93(9) |
| Br(1)-Fe(1)-Br(2) | 110.26(3) | 111.53(7) | 109.54(2) |

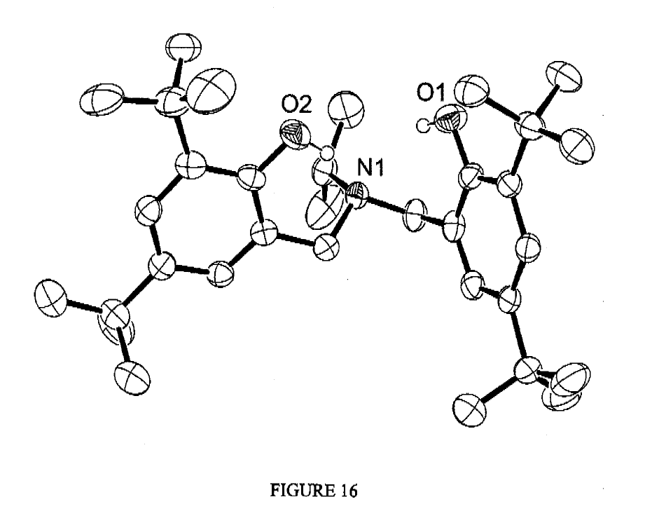

Single crystals of H₂L8 suitable for X-ray diffraction were obtained from a saturated methanol solution at −35° C. The solid state molecular structure and the crystallographic data of H₂L8 can be found in Table 21 and FIG. 16, respectively. Selected metric parameters for H₂L8 can be found in the electronic supplementary information. In the solid state, complexes 40 and 60 exhibit monomeric structures having tetrahedral iron(III) centres. Unlike complexes 10, 20 and 30, and also unlike the previously reported iron(III) complexes of amine-bis(phenolate) ligands (K. Hasan, C. Fowler, P. Kwong, A. K. Crane, J. L. Collins, C. M. Kozak, Dalton Trans., 2008, 2991; P. Mialane, E. Anxolabéhére-Mallart, G. Blondin, A. Nivorojkine, J. Guilhem, L. Tehertanova, M. Cesario, N. Ravi, E. Bominaar, J. Girerd, E. Munck, Inorg. Chim. Acta. 1997, 263, 367; and J. Strautmann, S. George, E. Bothe, E. Bill, T. Weyhermúller, A. Stammler, H. Bögge, T. Glaser, Inorg. Chem., 2008, 47, 6804), the bis(phenolate) ligand in 40 and 60 binds in a bidentate fashion. In both complexes (40 and 60), the central nitrogen donor is protonated giving a quaternized ammonium group. The oxygen donors of the phenolate groups remain anionic, giving a net monoanionic ammonium-bis(phenolate) ligand. Two bromide ions and the phenolate oxygen donor atoms make up the tetrahedral coordination environment about the iron(III) centre in both 40 and 60. The four-coordinate tetrahedral iron(III) centre is thereby formally anionic, resulting in an overall zwitterionic iron(III) complex. The bond angles around the metal range from 106.38(13)° to 110.90(9)° in 40, and 105.9(3)° to 112.0(2)° in 60, which are only moderately distorted from the ideal tetrahedral angle of 109.5°. The bond lengths of Fe—Br(1) and Fe—Br(2) are slightly asymmetrical in 40 and 60. The Fe—Br distances in 40 are 2.3596(9) and 2.3491(8) Å for Fe—Br(1) and Fe—Br(2) respectively, while the Fe—Br distances in 60 are 2.355(2) and 2.3697(19) Å for Fe—Br(1) and Fe—Br(2), respectively. The Fe—Br distances observed in 40 are slightly shorter than the terminally bonded Fe—Br bond length (2.3683(11) Å) found in a mononuclear square pyramidal iron(III) bromide complex (FeBr[O₂N₂]^{BuBu}) containing a salan ligand, previously reported in the Kozak group. (K. Hasan, C. Fowler, P. Kwong, A. K. Crane, J. L. Collins, C. M. Kozak, Dalton Trans., 2008, 2991) The Fe—Br distance of 2.3683(11) Å is intermediate to the Fe—Br bond lengths observed in the more sterically congested 60. In 40, the phenolate oxygen atoms exhibit bond distances to iron of 1.822(2) and 1.832(3) Å for Fe—O(1) and Fe—O(2), respectively. The Fe—O(1) and Fe—O(2) bond lengths observed in the related complex 60 (containing bulkier 2,4-di-tert-butylphenolate groups) are slightly longer, with distances of 1.843(6) and 1.851(6) Å, respectively. The Fe—O interactions observed in 40 and 60 are similar to those observed in FeBr[O₂N₂]^{BuBu}, where average Fe—O distances of 1.837 Å are observed (K. Hasan, C. Fowler, P. Kwong, A. K. Crane, J. L. Collins, C. M. Kozak, Dalton Trans., 2008, 2991).

The coordination geometry of 40 and 60 are similar to a tetrahedral iron(III) complex previously reported by Kozak and co-workers (X. Qian, L. Dawe, C. Kozak, Dalton Trans., 2010, 39, 1). Like 40, FeBr₂[O₂]^{BuMenPr} contains 2-tert-butyl-4-methylphenolate groups. However, unlike both 40 and 60, which possess an isopropyl alkyl group on the central nitrogen donor, the central nitrogen donor of FeBr₂[O₂]^{BuMenPr} contains a n-propyl alkyl substituent. Selected metric parameters for FeBr₂[O₂]^{BuMenPr} can be found in Table 24. As seen in Table 24, the Fe—O bond lengths observed in FeBr[O₂]^{BuMenPr} are slightly shorter than the corresponding Fe—O distances found in 60. However, the Fe—O bond lengths are very similar to those observed in 40. Similarly, as found in both 40 and 60, the Fe—Br bond lengths observed in FeBr₂[O₂]^{BuMenPr} are slightly asymmetrical. The Fe—Br(2) bond length (2.3723(7) Å) observed in FeBr₂[O₂]^{BuMenPr} is slightly longer than the Fe—Br distances found in 40 and 60. The bond angles around the iron centre range from 105.24(15)° to 112.87(10)° in FeBr₂[O₂]^{BuMenPr} and 106.38(13)° to 110.90(9)° in 40. Since both complexes share the same substituents on the phenolate rings, the differences in bond angles observed may be attributed to the differences in sterics originating from the alkyl substituents on the central nitrogen donor.

Previously, Leznoff and co-workers reported two different tetrahedral iron(III) bromide complexes which share a similar coordination geometry with 40 and 60 (A. Das, Z. Moatazedi, G. Mund, A. Bennet, I. Batchelor, D. B. Leznoff, Inorg. Chem. 2007, 46, 366; and G. Mund, D. Vidovic, R. Batchelor, J. Britten, R Sharma, C. Jones, D. B. Leznoff, Chem. Eur. J. 2003, 9, 4757). However, unlike the monomeric structure observed in 40 and 60, the iron (III) complexes {FeBr[MesN(SiMe₂)]₂O}₂ and {FeBr₂Li[Me₃PhN(SiMe₂)]₂O}₂ reported by the Leznoff group exhibit dimeric structures resulting in tetrahedral iron(III) centres bridged by bromide ligands. Compared to the bromide-bridged dimers reported by Leznoff and co-workers, 40 and 60 exhibit an unusual, neutral iron(III) dibromide tetrahedral environment. The Fe—Br distances observed in {FeBr[MesN(SiMe₂)]₂O}₂ and {FeBr₂Li[Me₃PhN(SiMe₂)]₂O}₂ are slightly longer than the Fe—Br bond lengths found in 40 and 60.

Molecular Structure of 50:

Although single crystals of 50 and their structure were also obtained, they were consistently of insufficient quality to provide data suitable for publication due to very weak diffraction at high angles (low cut-off in 20 (45°)). In the solid state, 50 exhibits a dimeric structure resulting in a trigonal bipyramidal iron(III) centre bridged by hydroxide ligands. A similar compound ([Fe[ONO]^{BuMeMe}(μ-OH)]₂) has been previously reported by Chaudhuri and co-workers (P. Chaudhuri, T. Weyhermüller, R. Wagner, Eur. J. Inorg. Chem. 2011, 2547). However, unlike 50, which contains 2,4-di-tert-butylphenolate groups, [Fe[ONO]^{BuMeMe}(μ-OH)]₂ possesses less sterically congested 2-tert-butyl-4-methylphenolate groups. In addition, the central nitrogen donors of [Fe[ONO]^{BuMeMe}(μ-OH)]₂ contain a methyl alkyl substituent, while the central nitrogen donors of 50 possess bulkier isopropyl alkyl groups. The Fe . . . Fe* distance of 3.13645(17) Å in 50, which is slightly longer than the Fe . . . Fe* distance (3.066 Å) observed in [Fe[ONO]^{BuMeMe}(μ-OH)]₂, precludes any bonding interaction between the metal centres. Like [Fe[ONO]^{BuMeMe}(μ-OH)]₂, two phenolate oxygen donor atoms and a bridging hydroxo oxygen atom occupy the equatorial plane around each iron ion in 50. The amine nitrogen and the bridging hydroxo oxygen atom O(3)* take up the axial positions in 50 and [Fe[ONO]^{BuMeMe}(μ-OH)]₂. The O(3)*-Fe—N bond angle in 50 and [Fe[ONO]^{BuMeMe}(μ-OH)]₂, is considerably distorted from the ideal linear geometry; it is bent away from the phenolate groups and directed towards the other bridging hydroxo group.

Magnetic Data for 50:

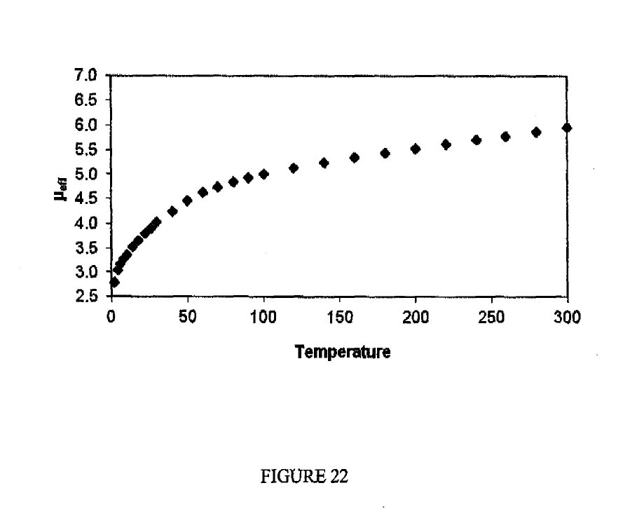

The temperature dependent magnetic behavior of 50 was examined in the temperature range of 2 to 300 K in an applied magnetic field of 1 T. The magnetic behavior of 50 is characteristic of an antiferromagnetically coupled dinuclear complex. Variable temperature magnetic studies show the μ_{eff} value of 5.96 μ_{B} at 300 K to decrease monotonically with decreasing temperature until it reaches a value of 2.79 μ_{B} at 2 K (FIG. 22). This suggests a small degree of exchange coupling between two paramagnetic high-spin iron(III) centres (S_{Fe}=5/2). Also, since there is no maximum observed in the plot of susceptibility, χ vs. T, the exchange coupling between the two metal centers would be very small. The moment at 2 K is higher than expected for a Sₜ=0 ground state and suggests the presence of a temperature independent paramagnetic impurity. Similar weak antiferromagnetic coupling has also been observed in similar dibridged diferric(III) complex previously reported in the Chaudhuri group which exhibits a rare case of exchange-coupled five-coordinate ferric(III) centres (P. Chaudhuri, T. Weyhermüller, R. Wagner, Eur. J. Inorg. Chem. 2011, 2547).

Conclusion

A series of iron(III) complexes supported by amine-bis(phenolate) ligands has been prepared. The five-coordinate complexes 10, 20 and 30 are monomeric in nature and exhibit mildly distorted trigonal bipyramidal coordination geometries. Unlike Complexes 10, 20 and 30, Complex 50 was determined to be dimeric in the solid state giving distorted trigonal bipyramidal iron(III) ions bridged by hydroxy ligands. The monomeric complexes 40 and 60 exhibit a tetrahedral coordination geometry where the iron(III) center is coordinated to two bromide ligands. The central nitrogen donors of 40 and 60 are protonated to give a quaternized ammonium fragment. Representative complexes 10, 20, 30, 40, 50 and 60 and ligand H₂L8, have been structurally characterized by single crystal X-ray diffraction. Additionally, all of the paramagnetic complexes 10, 20, 30, 40, 50 and 60 have been analytically verified by elemental analysis and MALDI-TOF mass spectrometry. Ligands H₂L6, H₂L7 and H₂L8 have been verified by ¹H-NMR, ¹³C-NMR and High-resolution mass spectrometry (HRMS).

The above described complexes are useful as cheap, relatively non-toxic catalysts for reactions in organic chemistry.

### Example 9

### Coupling of Benzyl Halide and Aryl Grignard Reagents Catalyzed by iron(III) amine-bisphenolate Complex 20

This example demonstrates the reaction of benzylamino-N,N-bis(2-methylene-4,6-di-tert-amylphenol), H₂L7, with anhydrous ferric chloride in the presence of a base yields FeCl(THF)L7 (20). In the solid state, complex 20 exists as a monomeric iron(III) species with a distorted trigonal bipyramidal geometry. Complex 20 is an air-stable, non-hygroscopic, single-component catalyst for C—C cross-coupling of aryl Grignard reagents with benzyl halides, including chlorides. Moderate to excellent yields of cross-coupled products can be obtained in diethyl ether at room temperature.

General Methods and Materials

All reagents were purchased either from Strem, Aldrich or Alfa Aesar and used without further purification. All C—C cross-coupling reactions were performed under an atmosphere of dry oxygen-free nitrogen by means of standard Schlenk techniques or by using an MBraun LabmasterDP glove box. Alkyl halides and Grignard reagents were purchased from Aldrich and used without further purification. Dodecane (Aldrich) was used as an internal standard for GC-MS analysis and diethyl ether was used for sample preparation. Acetophenone (Aldrich) was used as an internal standard for ¹H NMR analysis.

NMR spectra were recorded on a Bruker Avance III 300 MHz instrument with a 5 mm-multinuclear broadband observe (BBFO) probe. Gas chromatography mass spectrometry (GC-MS) analyses were performed using an Agilent Technologies 7890 GC system coupled to an Agilent Technologies 5975C mass selective detector (MSD). The chromatograph was equipped with electronic pressure control, split/splitless and on-column injectors, and an HP5-MS column.

The ligand H₂L7 and the complex 20 were prepared as described above in Example 8.

General Catalytic Method:

Catalyst 20 (0.10 mmol) in CH₂Cl₂ (3 mL) was added to a 30 mL Schlenk flask and the solvent removed in vacuo. Et₂O (5 mL) and the alkyl halide (2.0 mmol) were added to the catalyst under dry nitrogen. A solution of Grignard reagent (4.0 mmol) was added dropwise under vigorous stirring. The resulting mixture was stirred at room temperature for 30 min and the reaction was quenched with HCl (20 M, 5 mL). The organic phase was extracted with Et₂O (5 mL) and dried over MgSO₄. The organic phase was then passed through a plug of silica and the diethyl ether was removed in vacuo. The resulting products were then analyzed by GC-MS (using dodecane as internal standard) and NMR spectroscopy (using acetophenone as internal standard).

Results:

Previous studies with related Fe(III) complexes supported by tetradentate amine-bis(phenolate)-ether ligands suggest that diethyl ether is superior to THF as a solvent for the cross-coupling of Grignard reagents with alkyl halides (Chowdhury, R. R.; Crane, A. K.; Fowler, C.; Kwong, P.; Kozak, C. M. Chem. Commun. 2008, 94). In addition, it was previously found that reactions performed at room temperature gave superior results to those conducted at lower temperatures. Therefore, diethyl ether was the solvent of choice for the current study and all reactions were performed at room temperature. The first group of cross-coupling reactions investigated involved the reaction between benzyl bromide (or benzyl chloride) and a series of Grignard reagents (Table 25). An initial reaction of benzyl bromide with phenylmagnesium bromide (PhMgBr) in the presence of 20 gave a 30% yield of cross-coupled product after 30 minutes at room temperature (Table 25, entry 1). Significant yields of the bibenzyl and biaryl homocoupled by-products were also obtained. Benzyl chloride could also be used as the electrophilic partner, generating similar yields of the desired cross-coupled product (entry 2). The reaction of benzyl bromide with o-tolylmagnesium bromide gave a moderate yield (86%) of the cross-coupled product after 30 minutes at room temperature (entry 3). In a previous report, the reaction between benzyl bromide and o-tolylmagnesium bromide gave yields of 60% and 68% in the presence of octahedral (amine)bis(phenolato)Fe^{III}(acac) complexes and trigonal bipyramidal iron(III) halide complexes (supported by tetradentate amine-bis(phenolate) ligands), respectively (Chowdhury, R. R.; Crane, A. K.; Fowler, C.; Kwong, P.; Kozak, C. M. Chem. Commun. 2008, 94; and Hasan, K.; Dawe, L. N.; Kozak, C. M. Eur. J. Inorg. Chem. 2011, 4610). Surprisingly, when benzyl chloride was employed, a higher yield (94%) of the cross-coupled product was found (entry 4). A 95% yield of cross-coupled products was previously obtained from benzyl chloride and o-tolylmagnesium bromide in the presence of related tridentate amine-bis(phenolate) iron(III) complexes (Qian, X.; Dawe, L. N.; Kozak, C. M. Dalton Trans. 2011, 40, 933). A yield of 52% was reported in the presence of octahedral (amine)bis(phenolato)Fe^{III}acac) complexes (Hasan, K.; Dawe, L. N.; Kozak, C. M. Eur. J. Inorg. Chem. 2011, 4610). Using p-tolylmagnesium bromide, however, gave slightly lower yields than o-tolylmagnesium bromide with the respective benzyl halide (entries 5 and 6) generating higher yields of the bibenzyl and biaryl homocoupled by-products. A similar finding was also observed in the presence of octahedral (amine)bis(phenolato)Fe^{III}(acac) complexes and trigonal bipyramidal iron(III) chloride complexes with tetradentate amine-bis(phenolate) ligands (Chowdhury, R. R.; Crane, A. K.; Fowler, C.; Kwong, P.; Kozak, C. M. Chem. Commun. 2008, 94; and Hasan, K.; Dawe, L. N.; Kozak, C. M. Eur. J. Inorg. Chem. 2011, 4610). Bedford and co-workers reported the iron-catalyzed Negishi coupling of benzyl bromide with the diaryl zinc reagent prepared from p-tolylmagnesium bromide gave a 76% isolated yield of the desired cross-coupled product (Bedford, R. B.; Huwe, M.; Wilkinson, M. C. Chem. Commun. 2009, 600). When benzyl bromide was reacted with 4-methoxyphenylmagnesiumn bromide (4-anisylmagnesium bromide) in the presence of 20, a 21% yield of the cross-coupled product was found along with large quantities of the bibenzyl by-product (entry 7). The reaction between benzyl bromide and 4-anisylmagnesium bromide had been previously reported to result in a 0% yield of the cross-coupled product when iron(III) chloride complexes with tetradentate amine-bis(phenolate) ligands were used as the pre-catalyst (Chowdhury, R. R.; Crane, A. K.; Fowler, C.; Kwong, P.; Kozak, C. M. Chem. Commun. 2008, 94). The Negishi-type arylation between benzyl bromide and the corresponding diaryl zinc reagent prepared from 4-anisylmagnesium bromide resulted in a 95% isolated yield of the cross-coupled product (Bedford, R. B.; Huwe, M.; Wilkinson, M. C. Chem. Commun. 2009, 600). Reacting benzyl bromide with 4-fluorophenylmagnesium bromide (4-FPhMgBr) also resulted in a poor yield (21%) of the cross-coupled product (entry 8). High quantities of the homocoupled biaryl and bibenzyl products were formed instead. Surprisingly, benzyl bromide was found to couple with the sterically crowded 2,6-dimethylphenylmagnesium bromide (2,6-Me₂PhMgBr) in an excellent yield of 95% (entry 9). Previously, a 78% yield of cross-coupled product from benzyl bromide and 2,6-dimethylphenylmagnesium bromide was obtained when using a related tridentate amine-bis(phenolate) iron(III) complex (Qian, X.; Dawe, L. N.; Kozak, C. M. Dalton Trans. 2011, 40, 933). When benzyl bromide was reacted with the sp² hybridized vinylmagnesium bromide (in the presence of 20), very poor selectivity resulting in the formation of homo-coupled by-products was found. Only trace quantities of the desired product were obtained (entry 10) while high quantities of the bibenzyl by-product formed instead.

TABLE 25
The cross-coupling of benzyl bromide or benzyl chloride with Grignard reagents.

| | | | | | Yield^{b} | Yield |
|---|---|---|---|---|---|---|
| | | | | Yield^{a} | Ar—Ar | Bibenzyl |
| Entry | ArMgBr | Alkyl Halide | Product | (%) | (%) | (%) |
| 1 | Ph | 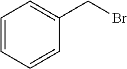 | 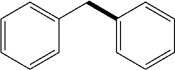 | 30 | 50 | 30 |
| 2 | Ph | 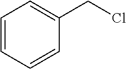 | 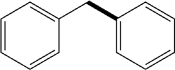 | 32 | 65 | <5 |
| 3 | o-tolyl | 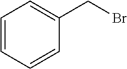 | 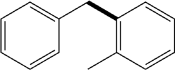 | 86 | 18 | 10 |
| 4 | o-tolyl | 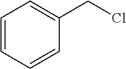 | 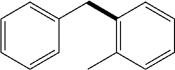 | 94 | 20 | <3 |
| 5 | p-tolyl | 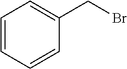 | 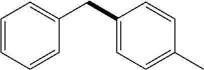 | 49 | 45 | 30 |
| 6 | p-tolyl | 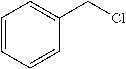 | 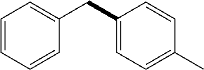 | 91 | 40 | 30 |
| 7 | 4-anisyl | 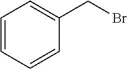 | 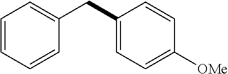 | 21 | 30 | 30 |
| 8 | 4-FPh | 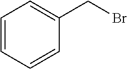 | 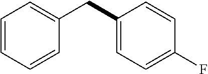 | 21 | 60 | 30 |
| 9 | 2,6- Me₂Ph | 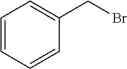 | 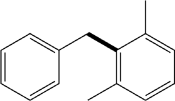 | 95 | <5 | trace |
| 10 | vinyl- MgBr | 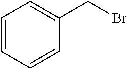 | 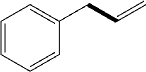 | trace | trace | 30 |

^{a}Spectroscopic yields determined by GC-MS using dodecane as an internal standard.
^{b}Percent yield of biaryl by-product is given with respect to alkyl halide.

A series of para-substituted benzyl halides were screened as a cross-coupling reaction partner. When 4-methylbenzyl bromide was reacted with 4-anisylmagnesiunm bromide in the presence of 20 at room temperature, a 19% yield of the desired cross-coupled product was obtained (Table 26, entry 1). High quantities of the homo-coupled bibenzyl product were formed instead. Reacting 4-methylbenzyl bromide with 4-fluorophenylmagnesium bromide (4-FPhMgBr) also resulted in a poor yield (13%) of the cross-coupled product (entry 2). When p-tolylmagnesium bromide was employed as the aryl Grignard reagent, a slightly higher yield of cross-coupled product (38%) was obtained (entry 3). However, high quantities of the bibenzyl and biaryl homocoupled by-products were generated. The Negishi-type arylation between 4-methylbenzyl bromide and the corresponding diaryl zinc reagent of p-tolylmagnesium resulted in an 86% isolated yield of the cross-coupled product as reported by Bedford and co-workers (Bedford, R. B.; Huwe, M.; Wilkinson, M. C. Chem. Commun. 2009, 600). Surprisingly, in the presence of 20, 4-methylbenzyl chloride was found to couple with p-tolylmagnesium in a high yield of 85% (entry 4). When the electron donating methyl group of 4-methylbenzyl bromide was replaced by a weakly electron withdrawing bromide group (4-methylbenzyl bromide), the desired cross-coupling product was obtained in a modest yield of 67% (entry 5) giving higher yields of the biphenyl homocoupled by-product. According to reports by Bedford et al., the iron-catalyzed Negishi coupling of 4-methylbenzyl bromide with the corresponding diaryl zinc reagent of p-tolylmagnesium bromide gave an 80% isolated yield of the desired cross-coupled product (Bedford, R. B.; Huwe, M.; Wilkinson, M. C. Chem. Commun. 2009, 600). Interestingly, when the strongly electron withdrawing substrate 4-(trifluoromethyl)benzyl bromide was employed, a higher yield (76%) of the cross-coupled product was obtained (entry 7). Bedford and co-workers found a 59% isolated yield of the cross-coupled product for the Negishi coupling of 4-(trifluoromethyl)benzyl bromide with the corresponding diaryl zinc reagent of p-tolylmagnesium bromide (Bedford, R. B.; Huwe, M.; Wilkinson, M. C. Chem. Commun. 2009, 600). As seen in Table 26, entry 6, the introduction of an ester group at the para position of the benzyl halide only gave trace quantities of the desired product resulting in the generation of high quantities of the homocoupled biaryl product instead.

TABLE 26
The cross-coupling of para-substituted benzyl halides with aryl Grignard reagents.

| | | | | | Yield^{b} | Yield |
|---|---|---|---|---|---|---|
| | | | | Yield^{a} | Ar—Ar | Bibenzyl |
| Entry | ArMgBr | Alkyl Halide | Product | (%) | (%) | (%) |
| 1 | 4-anisyl | 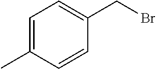 | 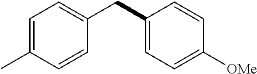 | 19 | 40 | 25 |
| 2 | 4-FPh | 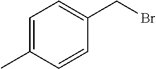 | 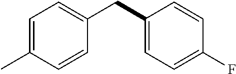 | 13 | 50 | 45 |
| 3 | p-tolyl | 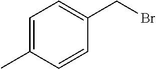 | 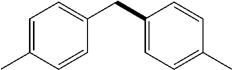 | 38 | 40 | 20 |
| 4 | p-tolyl | 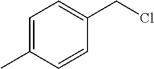 | 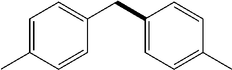 | 85 | 10 | 20 |
| 5 | p-tolyl | 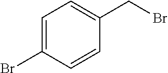 | 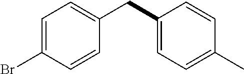 | 67 | 40 | 20 |
| 6 | p-tolyl | 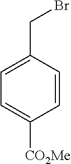 [SMILES: COC(=O)c1ccc(CBr)cc1] | 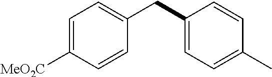 | trace | 50 | 0 |
| 7 | p-tolyl | 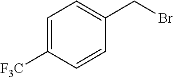 [SMILES: FC(F)(F)c1ccc(CBr)cc1] | 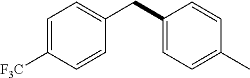 | 76 | 10 | 10 |

^{a}Spectroscopic yields determined by GC-MS using dodecane as an internal standard.
^{b}Percent yield of biaryl by-product is given with respect to alkyl halide.

Cross-coupling reactions with meta- and ortho-substituted benzyl halides were also screened. 3-Methoxybenzyl bromide was found to give low to modest yields depending on the aryl Grignard reagent used. In the presence of p-tolylmagnesium bromide, a moderate yield (72%) of the cross-coupled product was obtained (Table 27, entry 1). A higher yield of 92% was reported for the Negishi coupling of 3-methoxybenzyl bromide with the diaryl zinc reagent prepared from p-tolylmagnesium bromide (Bedford, R. B.; Huwe, M.; Wilkinson, M. C. Chem. Commun. 2009, 600). Surprisingly, 3-methoxybenzyl chloride could also be used as the electrophilic partner, generating a higher yield (91%) of the desired cross-coupled product (entry 2). Unfortunately, 3-methoxybenzyl bromide gave a low yield of the cross-coupled product when reacted with 4-anisylmagnesium bromide in the presence of 20 (entry 3). In fact, a high quantity of the unreacted starting material 3-methoxybenzyl bromide was found. When 2-bromobenzyl bromide was reacted with p-tolylmagnesium bromide in the presence of 20, only trace quantities of the desired cross-coupled product were generated with high yields of the biaryl homocoupled by-product (entry 4). When 2-(bromomethyl)benzonitrile was reacted with p-tolylmagnesium bromide, a 0% yield of the cross-coupled product was obtained (entry 5). In fact, the reaction selectively generated the biaryl homocoupled by-product. The reaction between 2-(trifluoromethyl)benzyl bromide and p-tolylmagnesium bromide gave a low yield (24%) of the cross-coupled product (entry 6). Low yields of the bibenzyl and biphenyl homocoupled by-products were also obtained. The Negishi coupling of 2-(trifluoromethyl)benzyl bromide with the corresponding diaryl zinc reagent of p-tolylmagnesium bromide gave a higher yield (64%) of the cross-coupled product (Bedford, R. B.; Huwe, M.; Wilkinson, M. C. Chem. Commun. 2009, 600).

TABLE 27
Cross-coupling of meta- and ortho-substituted benzyl halides with aryl Grignard
reagents.

| | | | | | Yield^{b} | Yield |
|---|---|---|---|---|---|---|
| | | Alkyl | | Yield^{a} | Ar—Ar | Bibenzyl |
| Entry | ArMgBr | Halide | Product | (%) | (%) | (%) |
| 1 | p-tolyl | 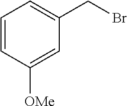 | 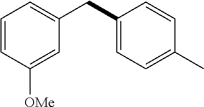 | 72 | 10 | 30 |
| 2 | p-tolyl | 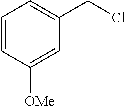 | 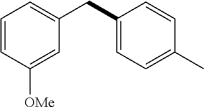 | 91 | 15 | 0 |
| 3 | 4-anisyl | 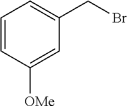 | 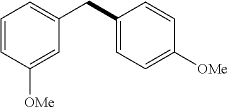 | 24 | 50 | 10 |
| 4 | p-tolyl | 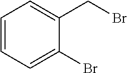 | 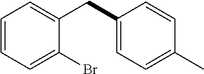 | trace | 70 | trace |
| 5 | p-tolyl | 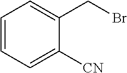 | 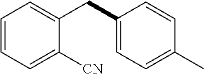 | 0 | 70 | 0 |
| 6 | p-tolyl | 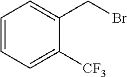 [SMILES: FC(F)(F)c1ccccc1CBr] | 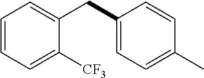 | 24 | 25 | 25 |

^{a}Spectroscopic yields determined by GC-MS using dodecane as an internal standard.
^{b}Percent yield of biaryl by-product is given with respect to alkyl halide.

Mechanistic Considerations

For many of the cross-coupling reactions attempted, bibenzyl homocoupling by-products were observed. Previously, for the reaction of dichloroethane with Grignard reagents, Hayashi and co-workers proposed a mechanism suggesting that benzyl halides could undergo radical reactions in the presence of reduced metals (Nagano, T.; Hayashi, T. Org. Lett. 2005, 7, 491). We also proposed a similar mechanism for the reaction between dichloromethane and Grignard reagents (Qian, X.; Kozak, C. M. Synlett 2011, 6, 852). Nakamura and Fürstner have also reported mechanisms where the iron-catalyzed cross-coupling of alkyl halides with aryl Grignard reagents proceeds via a radical process. (S. Groysman, I. Goldberg, M. Kol, E. Genizi, Z. Goldschmidt, Inorg. Chim. Acta. 2003, 345, 137; A. Philibert, F. Thomas, C. Philouze, S. Hamman, E. Saint-Aman, J. Pierre, Chem. Eur. J. 2003, 9, 3803) Benzyl halides can undergo oxidative addition (OA) at a reduced iron centre or undergo a single electron transfer (SET) reaction with the reduced centre generating an arylmethyl radical, which subsequently undergoes radical coupling (Scheme 11). (R. Chowdhury, A. Crane, C. Fowler, P. Kwong, C. Kozak, Chem. Commun. 2008, 94; P. Mialane, E. Anxolabéhére-Mallart, G. Blondin, A. Nivorojkine, J. Guilhem, L. Tehertanova, M. Cesario, N. Ravi, E. Bominaar, J. Girerd, E. Munck, Inorg. Chim. Acta. 1997, 263, 367) A similar mechanism may be responsible for the bibenzyl homocoupled byproduct observed in many of the cross-coupling reactions attempted and consequently the low yields of the desired cross-coupled product. As shown in Scheme 2, after the iron(III) pre-catalyst is reduced by the aryl Grignard reagent, the catalytically active iron species can either undergo oxidative addition (Path B) with the benzyl halide or take part in a single electron transfer (SET) side reaction (Path A) with the benzyl halide generating an arylmethyl radical, and in turn, 0.5 equivalents of the bibenzyl homocoupled by-product. If oxidative addition at the reduced iron centre occurs, the resulting benzylironhalide complex is expected to undergo transmetallation with the aryl Grignard to form an arylbenzyliron complex. Reductive elimination of the arylbenzyliron complex would then generate the desired cross-coupled product along with regeneration of the reduced iron species.

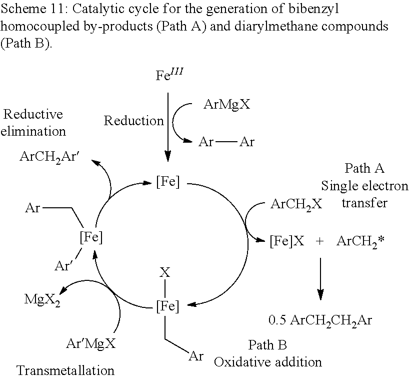

These investigations demonstrated that iron(III) complexes supported by tridentate amine-bis(phenolate) ligands catalyze the cross-coupling of aryl Grignard reagents with benzyl halides, although with highly variable yields and significant homo-coupled by-products. In the presence of 20, the coupling of o-tolylmagnesium bromide with benzyl halides (including chlorides) gave cross-coupled products in very high yields. The system also showed excellent reactivity for sterically demanding nucleophiles, such as 2,6-dimethylphenyl-magnesium bromide. Unfortunately, the reaction of the more sterically congested ortho-substituted benzyl halides with p-tolylmagnesium bromide gave very low yields of the desired cross-coupled product generating mainly homocoupled by-products.

All publications, patents and patent applications mentioned in this Specification are indicative of the level of skill of those skilled in the art to which this invention pertains and are herein incorporated by reference to the same extent as if each individual publication, patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. A compound of Formula I: 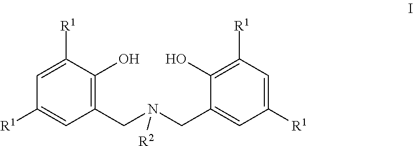
wherein
each R¹ is independently an electron withdrawing group; and
R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom,
with the proviso that when the R¹ substituents are all Cl, R² cannot be CH₃, CH₂CH₂N(CH₃)₂, or CH₂CH₂OCH₃, and when the R¹ substituents are all Br, R² cannot be CH₃ or CH₂CH₂N(CH₃)₂, and that when R² is CH₂(2-tetrahydrofuran) or CH₂CH₂methoxy, R¹ is an electron withdrawing group.

2. The compound of claim 1 wherein each R¹ is independently F, Cl, Br, I, CF₃, nitro, nitrile, carbonyl or a substituted carbonyl.

3. The compound of claim 1 or 2 wherein R² comprises a coordinating atom.

4. The compound of claim 3 wherein the coordinating atom is a Group 15 element, a Group 16 element or a carbenic atom of a carbene-containing moiety.

5. The compound of claim 3 or 4 wherein the coordinating atom is an aprotic N or O atom.

6. The compound of any one of claims 1-5 wherein R² is a dialkylaminoethyl, such as dimethylaminoethyl; tetrahydrofuranylethyl; pyridinylethyl; or alkoxyethyl, such as methoxyethyl or ethoxyethyl.

7. The compound of any one of claims 1-6 useful as tridentate ligand suitable for complexation with a metal.

8. The compound of any one of claims 1-6 useful as a tetradentate ligand suitable for complexation with a metal

9. The compound of any one of claims 1-8 suitable for complexation with iron.

10. A method for synthesizing a compound of Formula I, 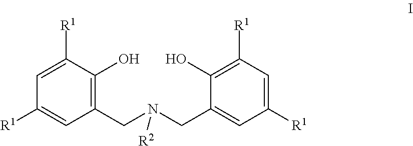
which comprises β-aminoalkylation of a phenol of Formula IV 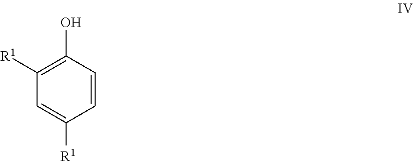
with formaldehyde and an amine of Formula V
H₂N—R² V
wherein
each R¹ is independently an electron withdrawing group; and
R² is H, a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom.

11. The method of claim 10 wherein the electron withdrawing group is selected from the group consisting of F, Cl, Br, I, CF₃, nitro, nitrile, carbonyl and substituted carbonyl.

12. The method of claim 10 or 11 wherein R² comprises a coordinating atom.

13. The method of claim 12 wherein the coordinating atom is a Group 15 element, a Group 16 element or a carbenic atom of a carbene-containing moiety.

14. The method of claim 12 or 13 wherein the coordinating atom is an aprotic N or O atom.

15. The method of any one of claims 10-14 wherein R² is dialkylaminoethyl, tetrahydrofuranylethyl, pyridinylethyl, or alkoxyethyl.

16. The method of any one of claims 10-15 wherein the method is carried out in water.

17. An iron complex having the structure of Formula II: 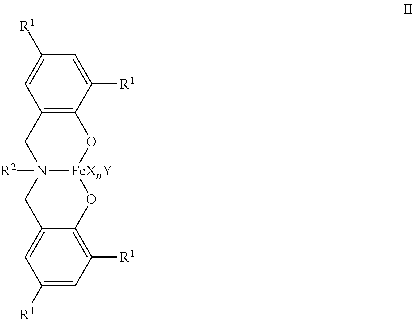
wherein
each R¹ is independently an electron withdrawing group or a substituted or unsubstituted C₁-C₂₅ linear, branched or cyclic alkyl, or a substituted or unsubstituted aryl, where R¹ does not comprise a coordinating atom;
R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom;
X is halogen, such as F, Cl, Br or I;
n is 1 or 2; and
Y is absent or a coordinating solvent molecule, or, when n is 2, Y is a negatively charged ionic species;
wherein when R² comprises the coordinating atom, the coordinating atom forms a dative bond to the Fe atom, and
with the proviso that when the R¹ substituents are all methyl, R² cannot be CH₂CH₂N(CH₃)₂ and that when the two ortho R¹ substituents are tert-butyl and the two para R¹ substituents are methyl, R² cannot be —CH₂pyridine or CH₂CH₂N(CH₃)₂, and that when R² is CH₂(2-tetrahydrofuran) or CH₂CH₂methoxy, R¹ is an electron withdrawing group.

18. The iron complex of claim 17 wherein the complex has the structure of Formula IIa or IIa′:

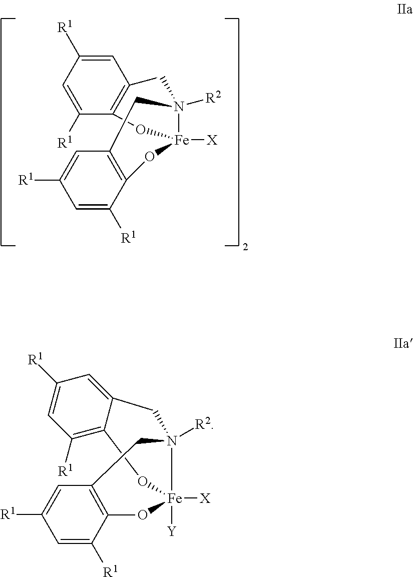

19. The iron complex of claim 17 wherein the complex has the structure of Formula

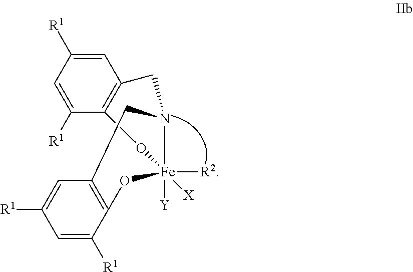

20. The iron complex of any one of claims 17-19, wherein the electron withdrawing group is selected from the group consisting of F, Cl, Br, I, CF₃, nitro, nitrile, carbonyl and substituted carbonyl.

21. The iron complex of any one of claims 17-20, wherein R² comprises a coordinating atom.

22. The iron complex of claim 21 wherein the coordinating atom is a Group 15 element, a Group 16 element or a carbenic atom of a carbene-containing moiety.

23. The iron complex of claim 21 or 22 wherein the coordinating atom is an aprotic N or O atom.

24. The iron complex of any one of claims 19-23 wherein R² is dialkylaminoethyl such as dimethylaminoethyl, tetrahydrofuranylethyl, pyridinylethyl, or alkoxyethyl such as methoxyethyl or ethoxyethyl.

25. A catalyst system comprising the iron complex of any one of claims 17-24.

26. The catalyst system of claim 25 further comprising one or more solvents, reagents, initiators, stabilizers, or combinations thereof.

27. A process for synthesizing an iron complex of Formula IIa, which comprises reacting an amine-bis(phenolate) ligand of Formula I with an iron halide to give the catalyst of Formula II: 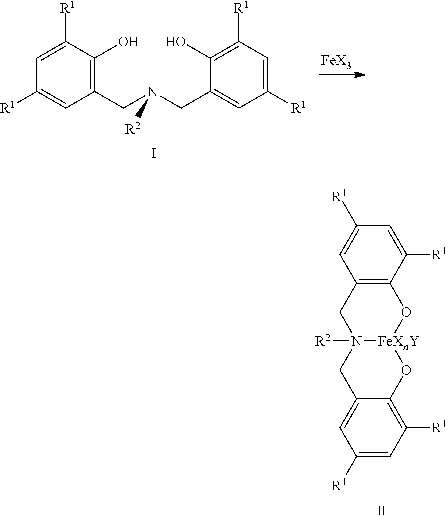
wherein
each R¹ is independently an electron withdrawing group or a substituted or unsubstituted C₁-C₂₅ linear, branched or cyclic alkyl, or a substituted or unsubstituted aryl, where R¹ does not comprise a coordinating atom;
R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom;
X is halogen, such as F, Cl, Br or I;
n is 1 or 2; and
Y is absent or a coordinating solvent molecule, or, when n is 2, Y is a negatively charged ionic species;
wherein when R² comprises a coordinating atom, the coordinating atom forms a dative bond to the Fe atom, and
with the proviso that when the R¹ substituents are all methyl, R² cannot be CH₂CH₂N(CH₃)₂ and that when the two ortho R¹ substituents are tert-butyl and the two para R¹ substituents are methyl, R² cannot be —CH₂pyridine or CH₂CH₂N(CH₃)₂, and that when R² is CH₂(2-tetrahydrofuran) or CH₂CH₂methoxy, R¹ is an electron withdrawing group.

28. The process of claim 27, wherein the amine-bis(phenolate) ligand of Formula I is tridentate to give the catalyst of Formula IIa or IIa′:

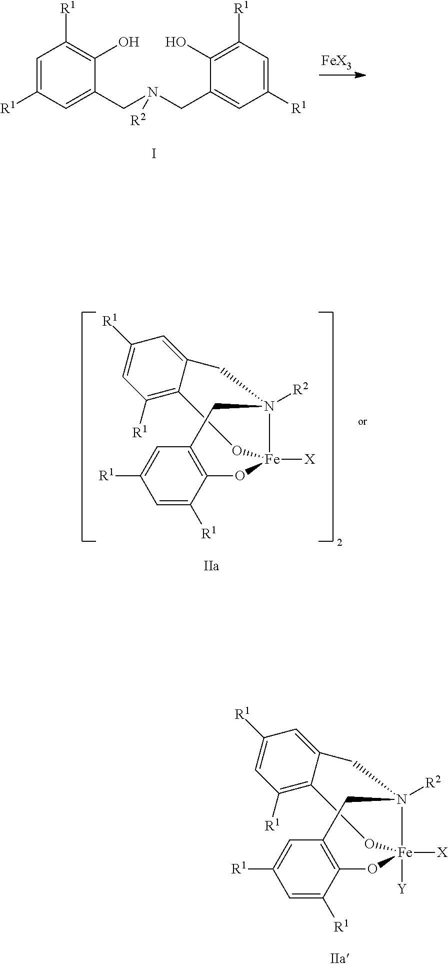

29. The process of claim 27, wherein the amine-bis(phenolate) ligand of Formula I is tetradentate to give the catalyst of Formula IIb:

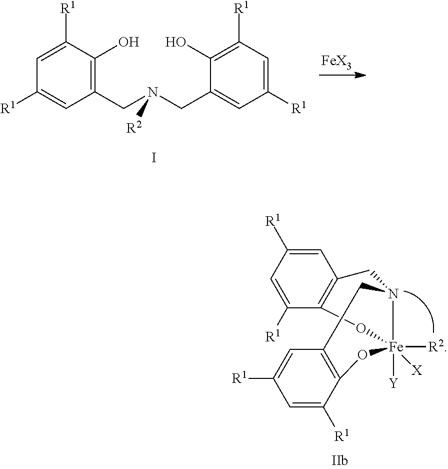

30. A method for cross coupling an alkyl or aryl Grignard reagent with a primary or secondary alkyl halide bearing a β-hydrogen, comprising reacting the Grignard reagent with the alkyl halide in the presence of an iron complex of Formula II 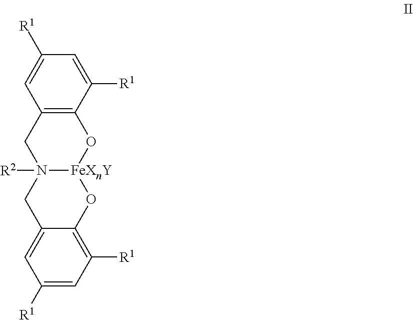
wherein
each R¹ is independently an electron withdrawing group or a substituted or unsubstituted C₁-C₂₅ linear, branched or cyclic alkyl, or a substituted or unsubstituted aryl, where R¹ does not comprise a coordinating atom;
R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom;
X is halogen, such as F, Cl, Br or I;
n is 1 or 2; and
Y is absent or a coordinating solvent molecule, or, when n is 2, Y is a negatively charged ionic species;
wherein when R² comprises a coordinating atom, the coordinating atom forms a dative bond to the Fe atom, with the proviso that when R² is CH₂(2-tetrahydrofuran) or CH₂CH₂methoxy, R¹ is an electron withdrawing group,
according to the following scheme:
R⁴—MgBr+R⁵—X¹→R⁴—R⁵
wherein
X¹ is an electronegative atom;
R⁴ is a C₁-C₂₅ substituted or unsubstituted, linear, branched or cyclic alkyl; a substituted or unsubstituted aryl; or a substituted or unsubstituted heterocyclic group; and
R⁵ is a C₁-C₂₅ substituted or unsubstituted, linear, branched or cyclic alkyl, or a C₂-C₂₅ substituted or unsubstituted, linear, branched or cyclic alkenyl or alkynyl; a substituted or unsubstituted aryl or a substituted or unsubstituted heterocyclic group.

31. The method of claim 30, wherein X¹ is Cl, Br or I.

32. The method of claim 30 or 31 wherein the iron complex has the structure of Formula IIa, IIa′, or IIb:

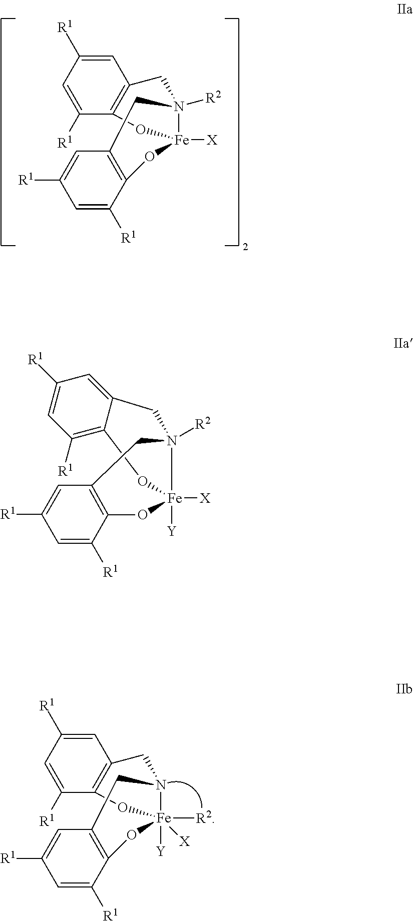

33. The method of any one of claims 30-32 wherein each R¹ is independently selected from the group consisting of F, Cl, Br, I, CF₃, nitro, nitrile, carbonyl and substituted carbonyl.

34. The method of claim 32 or 33 wherein R² comprises a coordinating atom.

35. The method of claim 34 wherein the coordinating atom is a Group 15 element, a Group 16 element or a carbenic atom of a carbene-containing moiety.

36. The method of claim 34 or 35 wherein the coordinating atom is an aprotic N or O atom.

37. The method of any one of claims 32-36 wherein R² is dialkylaminoethyl such as dimethylaminoethyl, tetrahydrofuranylethyl, pyridinylethyl, or alkoxyethyl such as methoxyethyl or ethoxyethyl.

38. The method according to any one of claims 30-37, wherein the method is performed at room temperature.

39. The method according to any one of claims 30-37, wherein the method is performed under heating, such as microwave heating.

40. The method according to any one of claims 30-39 wherein R⁴ is a substituted alkene.

41. A method for synthesizing a polymer by controlled radical polymerization, which comprises reacting a monomer and an initiator in the presence of an iron complex having the structure of Formula II 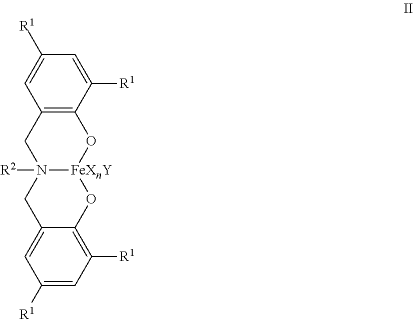
wherein
each R¹ is independently an electron withdrawing group or a substituted or unsubstituted C₁-C₂₅ linear, branched or cyclic alkyl, or a substituted or unsubstituted aryl;
R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom;
X is halogen, such as F, Cl, Br or I;
n is 1 or 2; and
Y is absent or a coordinating solvent molecule, or, when n is 2, Y is a negatively charged ionic species;
wherein when R² comprises a coordinating atom, the coordinating atom forms a dative bond to the Fe atom.

42. The method of claim 41 wherein the iron complex has the structure of Formula IIa or IIa′:

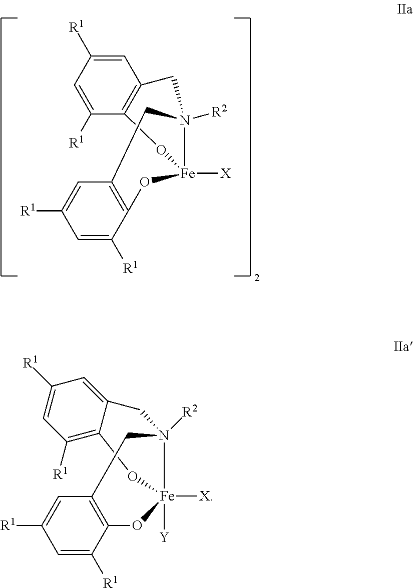

43. The method of claim 41 wherein the iron complex has the structure of Formula IIb:

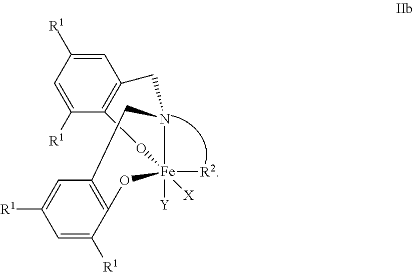

44. The method according to any one of claims 41-43, wherein each R¹ is independently a substituted or unsubstituted C₁-C₁₀ linear, branched or cyclic alkyl, F, Cl, Br, I, CF₃, nitro, nitrile, carbonyl or a substituted carbonyl.

45. The method according to any one of claims 41-44, wherein the monomer is selected from the group consisting of acrylates, methacrylates, styrenes, acrylonitriles, vinyl acetate, vinyl pyrrolidones, and combinations thereof.

46. The method according to any one of claims 41-45, wherein the monomer is selected from the group consisting of styrene, methyl methacrylate, methyl acrylate, vinyl acetate, and combinations thereof.

47. The method according to any one of claims 41-46, wherein the controlled radical polymerization is atom transfer radical polymerization.

48. The method according to any one of claims 41-47, wherein the initiator is Azobis(isobutyronitrile) (AIBN), V-65 or V-70.

49. A polymer synthesized by controlled radical polymerization, wherein the polymer contains at least trace amounts of iron or of the iron complex of Formula II.

50. The polymer of claim 49, wherein the controlled radical polymerization comprises the method of any one of claims 41 to 48.

51. The polymer according to claim 49 or 50 wherein the trace iron is iron oxide.

52. The polymer according to any one of claims 49-51 wherein the polymer is white.

53. The polymer according to any one of claims 49-52 wherein the polymer has a polydispersity index of 1.0-1.4.

54. The polymer according to any one of claims 49-53 wherein the polymer has a polydispersity index of 1.0-1.2.

55. A composition comprising a catalyst and a radical initiator, wherein the catalyst has the structure of Formula II 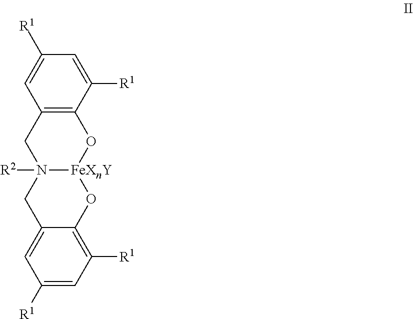
wherein
each R¹ is independently an electron withdrawing group or a substituted or unsubstituted C₁-C₂₅ linear, branched or cyclic alkyl, or a substituted or unsubstituted aryl;
R² is a substituted or unsubstituted C₁-C₂₅ linear, branched or cyclic alkyl, a substituted or unsubstituted non-aromatic heterocycle, a substituted or unsubstituted aryl, or a substituted or unsubstituted heteroaryl, and optionally comprises a coordinating atom;
X is halogen, such as F, Cl, Br or I;
n is 1 or 2; and
Y is absent or a coordinating solvent molecule, or, when n is 2, Y is a negatively charged ionic species;
wherein when R² comprises a coordinating atom, the coordinating atom forms a dative bond to the Fe atom.

56. The composition of claim 55, wherein the catalyst has the structure of Formula IIa or IIa′:

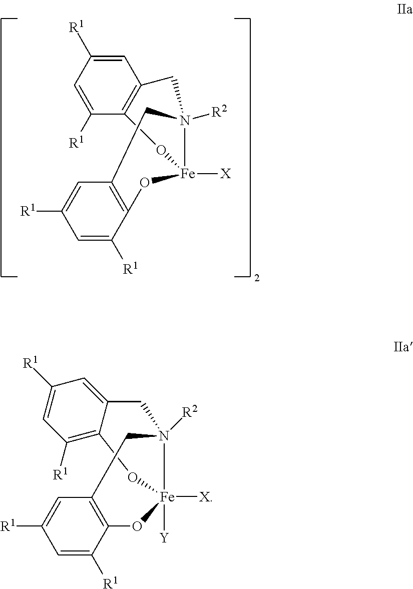

57. The composition of claim 55, wherein the catalyst has the structure of Formula IIb:

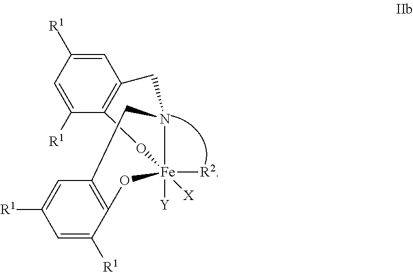

58. The composition according to any one of claims 55-57, wherein each R¹ is independently a substituted or unsubstituted C₁-C₁₀ linear, branched or cyclic alkyl, F, Cl, Br, I, CF₃, nitro, nitrile, carbonyl or a substituted carbonyl.

59. The composition according to any one of claims 55-58, wherein the monomer is selected from the group consisting of acrylates, methacrylates, styrenes, acrylonitriles, vinyl acetate, vinyl pyrrolidones, and combinations thereof.

60. The composition according to any one of claims 55-59, wherein the monomer is selected from the group consisting of styrene, methyl methacrylate, methyl acrylate, vinyl acetate, and combinations thereof.

61. The composition according to any one of claims 55-60, wherein the initiator is Azobis(isobutyronitrile) (AIBN), V-65 or V-70.
